SEQUENCE LISTING (21 sequences)

SEQ ID NO: 1 - DNA, 28 bp, Artificial
  Primer
cgagaatatgatagattatatgaagaat

SEQ ID NO: 2 - DNA, 30 bp, Artificial
  Primer
tggtttaatgctgttcatacgtttgtcaat

SEQ ID NO: 3 - DNA, 76 bp, Artificial
  Primer
gggacaagtttgtacaaaaaagcaggctacgaaggagatatacatatgcgagaatatgatagattatatgaagaat

SEQ ID NO: 4 - DNA, 66 bp, Artificial
  Primer
taccataattccaccaccaccaccggaaattccccctggtttaatgctgttcatacgtttgtcaat

SEQ ID NO: 5 - DNA, 26 bp, Artificial
  Primer
ctagtggaatgtttactaccaaatgg

SEQ ID NO: 6 - DNA, 26 bp, Artificial
  Primer
gttcaatgcatgctgtttaattgtgt

SEQ ID NO: 7 - DNA, 63 bp, Artificial
  Primer
gggggaatttccggtggtggtggtggaattatggtactagtggaatgtttactaccaaatgga

SEQ ID NO: 8 - DNA, 56 bp, Artificial
  Primer
agctccgtgatggtgatggtgatgtgctccgttcaatgcatgctgtttaattgtgt

SEQ ID NO: 9 - DNA, 61 bp, Artificial
  Primer
gggaccactttgtacaagaaagctgggtttaagctccgtgatggtgatggtgatgtgctcc

SEQ ID NO: 10 - DNA, 42 bp, Artificial
  Primer
gctagcatgcgagaatatgatagattatatgaagaatatacc

SEQ ID NO: 11 - DNA, 45 bp, Artificial
  Primer
gcctccaccacctccgcctggtttaatgctgttcatacgtttgtc

SEQ ID NO: 12 - DNA, 42 bp, Artificial
  Primer
tactagtccgcctccaccacctccgcctccaccacctccgcc

SEQ ID NO: 13 - DNA, 54 bp, Artificial
  Primer
actgaagcatcctcctcctcctcctcctggtttaatgctgttcatacgtttgtc

SEQ ID NO: 14 - DNA, 57 bp, Artificial
  Primer
agctccgtgatggtgatggtgatgtgctccagatctgtagtctttccgaactgtgtg

SEQ ID NO: 15 - DNA, 61 bp, Artificial; identical to SEQ ID NO: 9
  Primer

SEQ ID NO: 16 - DNA, 45 bp, Artificial
  Primer
tcctcctcctcctcctcctggtttaatgctgttcatacgtttgtc

SEQ ID NO: 17 - DNA, 26 bp, Artificial
  Primer
atgccccctggggtggactgccccat

SEQ ID NO: 18 - DNA, 26 bp, Artificial
  Primer
ctactgcctgttgtctttggacacgt

SEQ ID NO: 19 - DNA, 53 bp, Artificial
  Primer
attaaaccaggaggaggaggaggaggaccccctggggtggactgccccatgga

SEQ ID NO: 20 - DNA, 56 bp, Artificial
  Primer
agctccgtgatggtgatggtgatgtgctccctgcctgttgtctttggacacgttgt

SEQ ID NO: 21 - DNA, 61 bp, Artificial; identical to SEQ ID NO: 9
  Primer

(19) United States
(12) Patent Application Publication
(10) Pub. No.: US 2010/0305113 A1
(43) Pub. Date: Dec. 2, 2010

(54) Substituted Imidazopyridazines as Lipid Kinase Inhibitors

(76) Inventors: Hans-Georg Capraro, Basel (CH); Patricia Imbach, Basel (CH); Giorgio Caravatti, Basel (CH); Pascal Furet, Basel (CH)

Correspondence Address: NOVARTIS INSTITUTES FOR BIOMEDICAL RESEARCH, INC., 220 MASSACHUSETTS AVENUE, CAMBRIDGE, MA, 02139, US

(21) Appl. No.: 12/599,131

(22) PCT Filed: May 7, 2008
(86) PCT No.: PCT/EP2008/055636

§ 371 Date: Nov. 6, 2009

(30) Foreign Application Priority Data
May 9, 2007 (EP) 07107833.1

(51) Int. Cl. A61K 31/542; C07D 487/04; A61K 31/5025; A61K 31/5377; A61P 35/00; A61P 29/00; A61P 37/08; A61P 9/00

(52) U.S. Cl. 514/228.5; 544/236; 544/117; 544/61; 514/248; 514/233.2

(57) ABSTRACT

The invention relates to novel compounds of the formula I,

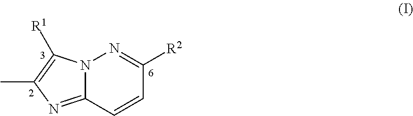

as well as other invention embodiments related to these compounds. The compounds are e.g. useful in the treatment of the animal or human body in view of their ability to inhibit protein kinases such as especially PI3 kinase.

The invention relates to novel 3-heterocyclyl-6-aryl-substituted 2-methyl-imidazo[1,2-b]pyridazines, processes for the preparation thereof, these compounds for use in the treatment of the human or animal body, the use thereof—alone or in combination with one or more other pharmaceutically active compounds—for the treatment (this term including prophylactic and/or therapeutic treatment) of an inflammatory or obstructive airway disease, such as asthma, disorders commonly occurring in connection with transplantation, or a proliferative disease, such as a tumor disease, which may be solid or liquid, especially one or more of the mentioned diseases which respond to an inhibition of kinases of the PI3-kinase-related protein kinase family, especially lipid kinases and/or PI3 kinase (PI3K) and/or mTOR and/or DNA protein kinase and/or ATM and/or ATR and/or hSMG-1 activity; a method for the treatment of such a disease in animals, especially a human, and the use of such a compound—alone or in combination with one or more other pharmaceutically active compounds—for the manufacture of a pharmaceutical preparation for the treatment of said diseases in animals, especially a human.

The 3-heterocyclyl-6-aryl-2-methyl-imidazo[1,2-b]pyridazines preferably are one or more compounds of the formula I,

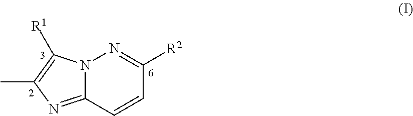

wherein
R¹ is unsubstituted or substituted aryl or heterocyclyl; and
R² is substituted phenyl or substituted naphthyl;
and/or an N-oxide thereof, a solvate and/or a (preferably pharmaceutically acceptable) salt thereof.

The general terms used hereinbefore and hereinafter preferably have within the context of this disclosure the following meanings, unless otherwise indicated, where more general terms wherever used may, independently of each other, be replaced by more specific definitions or remain, thus defining more preferred embodiments of the invention:

The prefix “lower” or “C₁-C₇-” denotes a radical having up to and including a maximum of 7, especially up to and including a maximum of 4 carbon atoms, the radicals in question being either linear or branched with single or multiple branching.

Lower alkyl (or C₁-C₇-alkyl) is preferably alkyl with from and including 1 up to and including 7, preferably from and including 1 to and including 4, and is linear or branched; preferably, lower alkyl is butyl, such as n-butyl, sec-butyl, isobutyl, tert-butyl, propyl, such as n-propyl or isopropyl, ethyl or preferably methyl.

The numbering of the positions of substituents at the central 2-methyl-imidazo[1,2-b]pyridazine ring system given in the present disclosure (e.g. in the Examples) is demonstrated in formula I above by the small numbers 2, 3 and 6.

Halogen, halogeno (or halo) is especially fluoro, chloro, bromo, or iodo, especially fluoro, chloro or bromo.

In unsubstituted or substituted alkyl, alkyl preferably has up to 20, more preferably up to 12, carbon atoms (also in alkyloxy) and is especially C₁-C₇-alkyl; is linear or branched one or more times; and is unsubstituted or substituted (in any, e.g. the terminal position) by one or more moieties selected from the substituents mentioned below for aryl, especially from halo and cyano.

Mono- or disubstituted amino is preferably amino substituted by unsubstituted or substituted alkyl as defined above, by unsubstituted or substituted cycloalkyl as defined below or by acyl (then preferably only with one acyl), such as C₁-C₇-alkanoyl, C₁-C₇-alkyloxycarbonyl, phenyl- and/or naphtyl-C₁-C₇-alkoxycarbonyl; preferred is N-mono- or N,N-di-(C₁-C₇-alkyl, hydroxyl-C₁-C₇-alkoxy-C₁-C₇-alkyl, phenyl-C₁-C₇-alkyl, naphthyl-C₁-C₇-alkyl, C₃-C₈-cycloalkyl, C₃-C₈-cycloalkyl-C₁-C₇-alkyl, C₁-C₇-alkanoyl, C₁-C₇-alkyloxycarbonyl, phenyl- and/or naphtyl-C₁-C₇-alkoxycarbonyl)-amino.

Mono- or di-substituted carbamoyl is preferably carbamoyl that is substituted by unsubstituted or substituted alkyl as defined above or by unsubstituted or substituted cycloalkyl as defined below; preferred is N-mono- or N,N-di-(C₁-C₇-alkyl, hydroxyl-C₁-C₇-alkyl, C₁-C₇-alkoxy-C₁-C₇-alkyl, phenyl-C₁-C₇-alkyl, naphthyl-C₁-C₇-alkyl, C₃-C₈-cycloalkyl and/or C₃-C₈-cycloalkyl-C₁-C₇-alkyl)-carbamoyl.

In unsubstituted or substituted heterocyclyl (also in unsubstituted or substituted heterocyclylcarbonyl (heterocyclyl-C(═O)—)), heterocyclyl is preferably a heterocyclic radical that is unsaturated (=carrying the largest possible number of conjugated double bonds in the ring(s), then heterocyclyl being heteroaryl), saturated or partially saturated and is preferably a monocyclic or in a broader aspect of the invention bicyclic or tricyclic ring; and has 3 to 24, more preferably 4 to 16, most preferably 4 to 10 and most preferably 5 or 6 ring atoms; wherein one or more, preferably one to four, especially one or two carbon ring atoms are replaced by a heteroatom selected from the group consisting of nitrogen, oxygen and sulfur, the bonding ring preferably having 4 to 12, especially 5 to 7 ring atoms; which heterocyclic radical (heterocyclyl) is unsubstituted or substituted by one or more, especially 1 to 3, substituents independently selected from the group consisting of the substituents defined below for substituted aryl; and where heterocyclyl is especially a heterocyclyl radical selected from the group consisting of oxiranyl, azirinyl, aziridinyl, 1,2-oxathiolanyl, thienyl (=thiophenyl), furanyl, tetrahydrofuryl, pyranyl, thiopyranyl, thianthrenyl, isobenzofuranyl, benzofuranyl, chromenyl, 2H-pyrrolyl, pyrrolyl, pyrrolinyl, pyrrolidinyl, imidazolyl, imidazolidinyl, benzimidazolyl, pyrazolyl, pyrazinyl, pyrazolidinyl, thiazolyl, isothiazolyl, dithiazolyl, oxazolyl, isoxazolyl, pyridinyl, pyrazinyl, pyrimidinyl, piperidinyl, piperazinyl, pyridazinyl, morpholinyl, thiomorpholinyl, (S-oxo or S,S-dioxo)-thiomorpholinyl, indolizinyl, azepanyl, diazepanyl, especially 1,4-diazepanyl, isoindolyl, 3H-indolyl, indolyl, benzimidazolyl, cumaryl, indazolyl, triazolyl, tetrazolyl, purinyl, 4H-quinolizinyl, isoquinolyl, quinolyl, tetrahydroquinolyl, tetrahydroisoquinolyl, decahydroquinolyl, octahydroisoquinolyl, benzofuranyl, dibenzofuranyl, benzothiophenyl, dibenzothiophenyl, phthalazinyl, naphthyridinyl, pyrrolo-pyrimidinyl, especially pyrrolo[2,3-d]pyrimidin-(e.g. 1-)yl, 1H,4H,5H-trihydropyrazolo[2,3-c]piperidin-1-yl, pyrrolo-pyridinyl, e.g. pyrrolo[2,3-c]pyridine-1-yl (meaning 5-aza-indol-1-yl), quinoxalyl, quinazolinyl, quinazolinyl, cinnolinyl, pteridinyl, carbazolyl, beta-carbolinyl, phenanthridinyl, acridinyl, perimidinyl, phenanthrolinyl, furazanyl, phenazinyl, phenothiazinyl, phenoxazinyl, chromenyl, isochromanyl, chromanyl, benzo[1,3]dioxol-5-yl and 2,3-dihydro-benzo[1,4]dioxin-6-yl, each of these radicals being unsubstituted or substituted by one or more, preferably up to three, substituents independently selected from those mentioned below for substituted aryl and from oxo, especially from the group consisting of C₁-C₇-alkyl that is unsubstituted or substituted by hydroxyl, by C₁-C₇-alkoxy, by halo, e.g. in trifluoromethyl, or by cyano-C₁-C₇-alkyl, e.g. hydroxy-C₁-C₇-alkyl, such as hydroxymethyl, or C₁-C₇-alkoxy-C₁-C₇-alkyl, such as methoxymethyl, from amino- or C₁-C₇-alkylamino-C₁-C₇-alkyl, halo, hydroxyl, (especially C₁-C₇-) alkoxy, oxo, amino, mono- or di-(C₁-C₇-alkyl, hydroxyl-C₁-C₇-alkyl and/or C₃-C₈-cyloalkyl)-amino, C₁-C₇-alkanoylamino, C₁-C₇-alkoxycarbonyl-amino, benzoylamino, aminobenzoylamino, C₁-C₇-alkoxycarbonylamino, (phenyl or naphthyl)-C₁-C₇-alkoxycarbonylamino, carbamoyl, N-mono or N,N-disubstituted carbamoyl, especially N-mono- or N,N-di-(C₁-C₇-alkyl, phenyl-C₁-C₇-alkyl and/or C₃-C₈-cycloalkyl)-aminocarbonyl, [heterocyclyl (especially pyrazolyl, such as pyrazolo, pyrrolidinyl, such as pyrrolidin-1-yl, pyridinyl, such as pyridin-(2-, 3- or 4-)yl, piperidinyl, such as piperidin-1-yl, oxopiperidinyl, such as 2-oxopiperidin-1-yl, piperazinyl, such as piperazin-1-yl, triazolyl, such as 1,2,4-triazol-1-yl, thiazolyl, morpholinyl, such as morpholino, thiomorpholinyl, such as thiomorpholino, S-oxothiomorpholinyl, such as S-oxothiomorpholino, benzimidazol (especially -1-)yl, pyrrolo-pyrimidinyl, especially pyrrolo[2,3-d]pyrimidin-(e.g. 1-)yl, or 1H,4H,5H-trihydropyrazolo[2,3-c]piperidin-1-yl) {wherein heterocyclyl is unsubstituted or substituted by one or more, especially up to three, substituents independently selected from C₁-C₇-alkyl, halo-C₁-C₇-alkyl, halophenyl, hydroxy, C₁-C₇-alkoxy, halo, C₁-C₇-alkoxycarbonyl, carbamoyl, phenylsulfonyl wherein phenyl is unsubstituted or substituted by one or more, preferably up to three, substituents independently selected from C₁-C₇-alkyl, hydroxy, C₁-C₇-alkoxy, halo, nitro and cyano, heterocyclylcarbonyl (=heterocyclyl-C(═O)—) where heterocyclyl is bound via a ring nitrogen to the carbonyl, especially piperidinocarbonyl, morpholino-carbonyl, thiomorpholino-carbonyl or S-oxo- or S,S-dioxothiomorpholinocarbonyl, C₁-C₇-alkanesulfonyl, such as methanesulfonyl, sulfamoyl, N-mono- or N,N-disubstituted sulfamoyl, preferably N-mono- or N,N-di-(C₁-C₇-alkyl)-sulfamoyl, cyano and nitro}]-aminocarbonyl, phenylaminocarbonyl, N—[N′-mono- or N′,N′-di-(C₁-C₇-alkyl)-amino-C₁-C₇-alkyl]-amino-carbonyl, mono- or di-[C₁-C₇-alkoxy, pyrrolidino, piperidino, piperazino, thiazolyl (e.g. thiazol-5-yl), hydroxyl-C₁-C₇-alkylamino and/or N′-mono- or N′,N′-di-(C₁-C₇-alkyl)-amino]-substituted phenyl-aminocarbonyl, heterocyclyl (especially pyrazolyl, pyrrolidinyl, pyridinyl, piperidinyl, oxopiperidinyl, piperazinyl, triazolyl, morpholinyl, thiomorpholinyl, S-oxothiomorpholinyl, benzimidazolyl, pyrrolo-pyrimidinyl, or 1H,4H,5H-trihydropyrazolo[2,3-c]piperidin-1-yl (meaning 5-aza-3,4,5,6-tetrahydroindazol-1-yl)) bound via a ring carbon atom or preferably a ring nitrogen and that is unsubstituted or substituted by one or more, especially up to three, substituents independently selected from C₁-C₇-alkyl, halophenyl, hydroxy, C₁-C₇-alkoxy, halo, C₁-C₇-alkoxycarbonyl, carbamoyl, phenylsulfonyl wherein phenyl is unsubstituted or substituted by one or more, preferably up to three, substituents independently selected from C₁-C₇-alkyl, hydroxy, C₁-C₇-alkoxy, halo, nitro and cyano, heterocyclylcarbonyl (=heterocyclyl-C(═O)—) where heterocyclyl is bound via a ring nitrogen to the carbonyl, especially piperidinocarbonyl, morpholino-carbonyl, thiomorpholino-carbonyl or S-oxo- or S,S-dioxothiomorpholinocarbonyl, C₁-C₇-alkanoyl, unsubstituted or substituted benzoyl wherein the substitutents are preferably one or more, e.g. up to three, substitutents independently selected from the group consisting of hydroxy, C₁-C₇-alkoxy and cyano, C₁-C₇-alkanesulfonyl, unsubstituted or substituted benzenesulfonyl wherein the substitutents are preferably one or more, e.g. up to three, substitutents independently selected from the group consisting of hydroxy, C₁-C₇-alkoxy and cyano, sulfamoyl, N-mono- or N,N-disubstituted sulfamoyl, preferably N-mono- or N,N-di-(C₁-C₇-alkyl)-sulfamoyl, cyano and nitro, and/or, in a broader aspect of the invention, further from unsubstituted or substituted aryl, from unsubstituted or substituted cycloalkyl and from unsubstituted or substituted heterocyclyl—especially in the case of unsubstituted or substituted heterocyclyl R¹ where the heterocyclyl substituents are preferably in the meta or para position relative to the binding aminocarbonyl group.

Unsubstituted or substituted heterocyclyl bound via an N-atom is preferably unsubstituted or substituted heterocyclyl as defined in the preceding paragraph which contains at least one nitrogen atom (which is preferably not charged without further protonation or N-oxide-formation) via which the respective moiety is bound to the rest of the molecule, especially one of the specific heterocyclyl moieties mentioned in the preceding paragraph wherein in the heterocyclic compound from which the moiety is formed by removal of a hydrogen from a ring NH a ring NH is present.

N-mono- or N,N-disubstituted sulfamoyl is preferably sulfamoyl that is substituted by unsubstituted or substituted alkyl as defined above or by unsubstituted or substituted cycloalkyl as defined below; preferred is N-mono- or N,N-di-(C₁-C₇-alkyl, hydroxyl-C₁-C₇-alkyl, C₁-C₇-alkoxy-C₁-C₇-alkyl, phenyl-C₁-C₇-alkyl, naphthyl-C₁-C₇-alkyl, C₃-C₈-cycloalkyl and/or C₃-C₈-cycloalkyl-C₁-C₇-alkyl)-sulfamoyl.

Unsubstituted or substituted cycloalkyl is preferably a cycloalkyl which has 3 to 18, more preferably 3 to 10, most preferably 3 to 8 ring carbon atoms and is unsubstituted or substituted by one or more, especially up to 3, more preferably one or two, substitutents independently selected from those given below for substituted aryl.

In unsubstituted or substituted aryl, aryl preferably has 6 to 18 carbon atoms and is a mono-, di- or polycyclic (preferably up to tricyclic, more preferably up to bicyclic) unsaturated carbocyclic moiety with conjugated double bonds in the ring, especially phenyl, naphthyl, biphenylenyl, indacenyl, acenaphthylenyl, fluorenyl, phenalenyl, phenanthrenyl or anthracenyl. Naphthyl and preferably phenyl are especially preferred. Aryl is unsubstituted or (in the case of substituted aryl) substituted by one or more, e.g. one to three, substitutents preferably independently selected from the group consisting of C₁-C₇-alkyl, such as methyl, ethyl, n-propyl, isopropyl, n-butyl, isobutyl, sec-butyl or tert-butyl; C₂-C₇-alkenyl; C₂-C₇-alkinyl; C₅-C₁₈-aryl-C₁-C₇-alkyl in which aryl is preferably phenyl, naphthyl, biphenylenyl, indacenyl, acenaphthylenyl, fluorenyl, phenalenyl, phenanthrenyl or anthracenyl and is unsubstituted or substituted by C₁-C₇-alkyl, such as methyl or ethyl, by pyrrolidinyl, especially pyrrolidino, by piperazinyl, especially piperazino, by amino, by N-mono- and/or N,N-di-C₁-C₇-alkylamino, by halo, by hydroxy, by C₁-C₇-alkoxy, such as methoxy, and/or by halo-C₁-C₇-alkyl, such as trifluoromethyl; [pyrrolidinyl (especially pyrrolidino), piperidinyl (especially piperidino), piperazinyl (especially piperazino), morpholino, thiomorpholino, pyridinyl, pyrimidinyl, pyrazinyl, pyridazinyl, oxazolyl or thiazolyl]-C₁-C₇-alkyl wherein pyrrolidinyl, piperidinyl, piperazinyl, pyridinyl, pyrimidinyl, pyrazinyl, pyridazinyl, oxazolyl or thiazolyl are unsubstituted or substituted by C₁-C₇-alkyl, such as methyl or ethyl, by pyrrolidinyl, especially pyrrolidino, by piperazinyl, especially piperazino, by amino, by N-mono- and/or N,N-di-C₁-C₇-alkylamino, by halo, by hydroxyl, by C₁-C₇-alkoxy, such as methoxy, by oxo and/or by halo-C₁-C₇-alkyl, such as trifluoromethyl, for example pyrrolidino-C₁-C₇-alkyl, 2-oxopyrrolidino-C₁-C₇-alkyl piperidino-C₁-C₇-alkyl, morpholino-C₁-C₇-alkyl, thiomorpholino-C₁-C₇-alkyl, N—C₁-C₇-alkyl-piperazino-C₁-C₇-alkyl, or N-mono- or N,N-di-(C₁-C₇-alkyl)-amino-substituted or unsubstituted pyrrolidino-C₁-C₇-alkyl; [pyrrolidinyl (especially pyrrolidino), piperidinyl (especially piperidino), piperazinyl (especially piperazino), pyridinyl, pyrimidinyl, pyrazinyl, pyridazinyl, oxazolyl or thiazolyl]-oxy-C₁-C₇-alkyl wherein pyrrolidinyl, piperidinyl, piperazinyl, pyridinyl, pyrimidinyl, pyrazinyl, pyridazinyl, oxazolyl and thiazolyl are unsubstituted or substituted by C₁-C₇-alkyl, such as methyl or ethyl, by pyrrolidinyl, especially pyrrolidino, by piperazinyl, especially piperazino, by amino, by N-mono- and/or N,N-di-C₁-C₇-alkylamino, by halo, by hydroxyl, by C₁-C₇-alkoxy, such as methoxy, by oxo and/or by halo-C₁-C₇-alkyl, such as trifluoromethyl; [pyrrolidin (especially pyrrolidino), piperidin (especially piperidino), piperazin (especially piperazino), pyridin, pyrimidin, pyrazin, pyridazin, oxazoly or thiazol]-carbonyl-C₁-C₇-alkyl wherein pyrrolidin, piperidin, piperazin, pyridin, pyrimidin, pyridazin, oxazol or pyridazin are unsubstituted or substituted by C₁-C₇-alkyl, such as methyl or ethyl, by pyrrolidinyl, especially pyrrolidino, by piperazinyl, especially piperazino, by amino, by N-mono- and/or N,N-di-C₁-C₇-alkylamino, by halo, by hydroxyl, by C₁-C₇-alkoxy, such as methoxy, by oxo and/or by halo-C₁-C₇-alkyl, such as trifluoromethyl; halo-C₁-C₇-alkyl, such as trifluoromethyl; hydroxy-C₁-C₇-alkyl, such as hydroxymethyl; C₁-C₇-alkoxy-C₁-C₇-alkyl, such as 3-methoxypropyl or 2-methoxyethyl; C₁-C₇-alkoxy-C₁-C₇-alkoxy-C₁-C₇-alkyl; phenyloxy- or naphthyloxy-C₁-C₇-alkyl; phenyl-C₁-C₇-alkoxy- or naphthyl-C₁-C₇-alkoxy-C₁-C₇-alkyl; amino-C₁-C₇-alkyl, such as aminomethyl; N-mono- or N,N-di-(C₁-C₇-alkyl, C₁-C₇-alkoxy-C₁-C₇-alkyl and/or (mono- or di-(C₁-C₇-alkyl)-amino)-C₁-C₇-alkyl)-amino-C₁-C₇-alkyl; C₁-C₇-alkoxy-C₁-C₇-alkylamino-C₁-C₇-alkyl; mono- or di-[C₆-C₁₈-aryl]-C₁-C₇-alkyl in which aryl is preferably phenyl, naphthyl, biphenylenyl, indacenyl, acenaphthylenyl, fluorenyl, phenalenyl, phenanthrenyl or anthracenyl and unsubstituted or substituted by C₁-C₇-alkyl, such as methyl or ethyl, by pyrrolidinyl, especially pyrrolidino, by piperazinyl, especially piperazino, by amino, by N-mono- and/or N,N-di-C₁-C₇-alkylamino, by halo, by hydroxyl, by C₁-C₇-alkoxy, such as methoxy, and/or by halo-C₁-C₇-alkyl, such as trifluoromethyl; (naphthyl- or phenyl-C₁-C₇-alkyl)-amino-C₁-C₇-alkyl; C₁-C₇-alkanoylamino-C₁-C₇-alkyl; carboxy-C₁-C₇-alkyl; benzoyl- or naphthoylamino-C₁-C₇-alkyl; C₁-C₇-alkylsulfonylamino-C₁-C₇-alkyl; phenyl- or naphthylsulfonylamino-C₁-C₇-alkyl wherein phenyl or naphthyl is unsubstituted or substituted by one or more, especially one to three, C₁-C₇-alkyl moieties; phenyl- or naphthyl-C₁-C₇-alkylsulfonylamino-C₁-C₇-alkyl; cyano-C₁-C₇-alkyl; halo, especially fluoro (preferred), chloro (preferred) or bromo; hydroxy; C₁-C₇-alkoxy, such as methoxy, ethoxy or propoxy, each of which is unsubstituted or substituted by one or more substituents selected from pyrrolidinyl, especially pyrrolidino, by piperazinyl, especially piperazino, by amino, by N-mono- and/or N,N-di-C₁-C₇-alkylamino, by halo, by hydroxyl, by C₁-C₇-alkoxy, such as methoxy, by halo-C₁-C₇-alkyl, such as trifluoromethyl and/or by a cyclic ether radical such as oxiranyl, oxetanyl, tetrahydrofuranyl or tetrahydropyranyl, especially oxetan-2-yl or oxetan-3-yl, with each cyclic ether radical being unsubstituted or substituted at the same carbon which is attached to said C₁-C₇-alkoxy group (i.e. forming e.g. an oxetan-3-diyl radical in the case of oxetan-3-yl being substituted at the 3-position) with a substituent independently selected from, pyrrolidinyl, especially pyrrolidino, by piperazinyl, especially piperazino, by amino, by N-mono- and/or N,N-di-C₁-C₇-alkylamino, N-mono- and/or N,N-di-C₁-C₇-alkanecarbonylamino, (e.g methyl-, ethyl-, propyl-, isopropylcarboxamido), N-mono- and/or N,N-di-C₃-C₇-cycloalkanecarbonylamino (e.g. cyclopropylcarboxamido), N-mono- and/or N,N-di-C₁-C₇-halo-alkanecarbonylamino (e.g. trifluoromethylcarboxamido), N-mono- and/or N,N-di-C₁-C₇-alkanoxycarbonylamino (e.g. methoxycarbonylamino, tert-butyloxycarbonylamino and the like), wherein the alkyl group of the N-mono- and/or N,N-di-C₁-C₇-alkanoxycarbonylamino radical is unsubstituted or substituted by aryl, especially phenyl, naphthyl, biphenylenyl, indacenyl, acenaphthylenyl, fluorenyl, phenalenyl, phenanthrenyl or anthracenyl (e.g. giving benzyloxycarbonylamino when the N-mono- and/or N,N-di-C₁-C₇-alkanoxycarbonylamino radical is methoxycarbonylamino and the methyl group thereof is substituted by aryl which is phenyl), pyrrolidinyl, especially pyrrolidino, by piperazinyl, especially piperazino, by amino, by N-mono- and/or N,N-di-C₁-C₇-alkylamino, by halo, by hydroxyl, by C₁-C₇-alkoxy, such as methoxy, and/or by halo-C₁-C₇-alkyl, such as trifluoromethyl, by halo, by hydroxyl, by C₁-C₇-alkoxy, such as methoxy, by halo-C₁-C₇-alkyl, such as trifluoromethyl; C₆-C₁₈-aryl-C₁-C₇-alkoxy in which aryl is preferably phenyl, naphthyl, biphenylenyl, indacenyl, acenaphthylenyl, fluorenyl, phenalenyl, phenanthrenyl or anthraxcenyl and unsubstituted or substituted by C₁-C₇-alkyl, such as methyl or ethyl, by C₁-C₇-alkoxy, by pyrrolidinyl, especially pyrrolidino, by piperazinyl, especially piperazino, by amino, by N-mono- and/or N,N-di-C₁-C₇-alkylamino, by halo, by hydroxyl, by C₁-C₇-alkoxy, such as methoxy, and/or by halo-C₁-C₇-alkyl, such as trifluoromethyl; hydroxy-C₁-C₇-alkoxy; C₁-C₇-alkoxy-C₁-C₇-alkoxy; C₁-C₇-alkoxy-C₁-C₇-alkoxy-C₁-C₇-alkoxy; halo-C₁-C₇-alkoxy; amino-C₁-C₇-alkoxy; N-mono- or N,N-di-(C₁-C₇-alkyl)-amino-C₁-C₇-alkoxy; N—C₁-C₇-alkanoylamino-C₁-C₇-alkoxy; C₁-C₇-alkoxycarbonylamino-C₁-C₇-alkoxy; C₆-C₁₄-arylcarbonylamino-C₂-C₇-alkoxy (C₆-C₁₄-aryl-C(═O)—NH—C₂-C₇-alkoxy or C₆-C₁₄-aroyl-NH—C₂-C₇-alkoxy) wherein C₆-C₁₄-aryl is unsubstituted or substituted by one or more, especially up to three, substituents independently selected from the group consisting of C₁-C₇-alkyl, halo-C₁-C₇-alkyl, hydroxy, C₁-C₇-alkoxy, halo and cyano; N-unsubstituted-, N-mono- or N,N-di-(C₁-C₇-alkyl)carbamoyl-C₁-C₇-alkoxy; phenyl- or naphthyloxy; phenyl- or naphthyl-C₁-C₇-alkyloxy; [pyrrolyl, pyrrolidinyl (especially pyrrolidino), imidazolyl (especially imidazolo), imidazolidinyl (especially imidazolidino), piperidinyl (especially piperidino), piperazinyl (especially piperazino), pyridinyl, pyrimidinyl, pyrazinyl, pyridazinyl, oxazolyl, thiazolyl, morpholinyl (especially morpholino), thiomorpholinyl (especially thiomorpholino), S-oxothiomorpholinyl (especially S-oxothiomorpholino) or S,S-dioxothiomorpholinyl (especially S,S-dioxothiomorpholino)]-C₁-C₇-alkoxy wherein pyrrolidinyl, piperidinyl, piperazinyl, pyridinyl, pyrimidinyl, pyrazinyl, pyridazinyl, oxazolyl and thiazolyl are unsubstituted or substituted by C₁-C₇-alkyl, such as methyl or ethyl, by pyrrolidinyl, especially pyrrolidino, by piperazinyl, especially piperazino, by amino, by N-mono- and/or N,N-di-C₁-C₇-alkylamino, by halo, by hydroxyl, by C₁-C₇-alkoxy, such as methoxy, by oxo and/or by halo-C₁-C₇-alkyl, such as trifluoromethyl; [pyrrolyl, pyrrolidinyl (especially pyrrolidino), imidazolyl (especially imidazolo), imidazolidinyl (especially imidazolidino), piperidinyl (especially piperidino), piperazinyl (especially piperazino), pyridinyl, pyrimidinyl, pyrazinyl, pyridazinyl, oxazolyl, thiazolyl, morpholinyl (especially morpholino), thiomorpholinyl (especially thiomorpholino), S-oxothiomorpholinyl (especially S-oxothiomoprpholino) or S,S-dioxothiomorpholinyl (especially S,S-dioxothiomorpholino)]oxy-C₁-C₇-alkoxy wherein pyrrolidinyl, piperidinyl, piperazinyl, pyridinyl, pyrimidinyl, pyrazinyl, pyridazinyl, oxazolyl and thiazolyl are unsubstituted or substituted by C₁-C₇-alkyl, such as methyl or ethyl, by pyrrolidinyl, especially pyrrolidino, by piperazinyl, especially piperazino, by amino, by N-mono- and/or N,N-di-C₁-C₇-alkylamino, by halo, by hydroxyl, by C₁-C₇-alkoxy, such as methoxy, by oxo and/or by halo-C₁-C₇-alkyl, such as trifluoromethyl; C₃-C₈-cyloalkoxy; pyridincarbonylamino-C₁-C₇-alkoxy, C₆-C₁₄-arylaminocarbonylamino-C₂-C₇-alkoxy (C₆-C₁₄-aryl-NH—C(═O)—NH—C₂-C₇-alkoxy) wherein C₆-C₁₄-aryl is unsubstituted or substituted by one or more, especially up to three, substituents independently selected from the group consisting of C₁-C₇-alkyl, halo-C₁-C₇-alkyl, hydroxy, C₁-C₇-alkoxy, halo and cyano; pyridinylaminocarbonylamino-C₁-C₇-alkoxy; C₁-C₇-alkanoyloxy; benzoyl- or naphthoyloxy; amino; mono- or di-(C₁-C₇-alkyl, C₃-C₈-cyloalkyl and/or hydroxyl-C₁-C₇-alkyl)-amino; mono- or di-(naphthyl- or phenyl-C₁-C₇-alkyl)-amino; C₁-C₇-alkanoylamino; unsubstituted or amino-, N-mono- or N,N-di-(C₁-C₇-alkyl and/or phenyl- or naphthyl-C₁-C₇-alkyl)amino-substituted benzoyl- or naphthoylamino; C₁-C₇-alkoxycarbonylamino; (phenyl or naphthyl)-C₁-C₇-alkoxycarbonylamino; C₁-C₇-alkylsulfonylamino; phenyl- or naphthylsulfonylamino wherein phenyl or naphthyl is unsubstituted or substituted by one or more, especially one to three, C₁-C₇-alkyl moieties; phenyl- or naphthyl-C₁-C₇-alkylsulfonylamino; C₁-C₇-alkanoyl; unsubstituted or substituted benzoyl wherein the substitutents are preferably one or more, e.g. up to three, substitutents independently selected from the group consisting of hydroxy, C₁-C₇-alkoxy and cyano; C₁-C₇-alkylthio; halo-C₁-C₇-alkylthio, such as trifluoromethylthio; C₁-C₇-alkane-sulfonyl; C₃-C₈-cyloalkyl-sulfonyl; C₁-C₇-alkoxy-C₁-C₇-alkylthio; phenyl- or naphthylthio; phenyl- or naphthyl-C₁-C₇-alkylthio; C₁-C₇-alkanoyithio; benzoyl- or naphthaylthio; C₁-C₇-alkanoyl; C₁-C₇-alkoxy-C₁-C₇-alkanoyl; carboxyl (—COOH); C₁-C₇-alkoxy-carbonyl; phenoxy- or naphthoxycarbonyl; phenyl- or naphthyl-C₁-C₇-alkoxycarbonyl; C₁-C₁₀-especially C₁-C₄-alkylendioxy, such as methylendioxy or 1,2-ethylendioxy; carbamoyl; N-mono- or N,N-di-[C₁-C₇-alkyl, naphthyl-C₁-C₇-alkyl, phenyl-C₁-C₇-alkyl, N′-mono- or N′,N′-di-(C₁-C₇-alkyl)amino-C₁-C₇-alkyl, pyrrolidinyl (especially pyrrolidino)-C₁-C₇-alkyl, piperidinyl (especially piperidino)-C₁-C₇-alkyl, piperazinyl- or N—(C₁-C₇-alkyl)piperazinyl (especially piperazino or 4-C₁-C₇-alkylpiperazino)-C₁-C₇-alkyl, mono-C₁-C₇-alkoxy-C₁-C₇-alkyl, (N′-mono- or N′,N′-di-(C₁-C₇-alkyl)-amino)-C₁-C₇-alkyl, phenyl, pyridinyl, oxazolyl or thiazolyl each of which is unsubstituted or substituted by C₁-C₇-alkoxy, by halo, especially fluoro, by pyrrolidino, by piperidino, by piperazino, by hydroxyl-C₁-C₇-alkylamino, by hydroxyl-C₁-C₇-alkyl, by amino or by N-mono- or N,N-di-(C₁-C₇-alkyl)amino, C₃-C₈-cyloalkyl, pyrrolidinyl, piperidinyl, morpholinyl, piperazinyl, pyrimidinyl, pyrazinyl and/or pyridazinyl]-amino-carbonyl, such as N-mono- or N,N-di-(C₁-C₇-alkyl)-aminocarbonyl; N—C₁-C₇-alkoxy-C₁-C₇-alkylcarbamoyl; pyrrolidin-1-carbonyl; amino-N-pyrrolidin-1-carbonyl; N-mono- or N,N-di(C₁-C₇-alkyl)amino-pyrrolidin-1-carbonyl; piperidin-1-carbonylmorpholin-4-carbonyl; morpholinocarbonyl, thiomorpholinocarbonyl, S-oxo- or S,S-dioxo-thiomorpholino-carbonyl, thiomorpholin-4-carbonyl; S-oxo-thiomorpholin-4-carbonyl; S,S-dioxothiomorpholin-4-carbonyl; piperazin-1-carbonyl; N—C₁-C₇-alkyl-piperazin-1-carbonyl; N—C₁-C₇-alkoxycarbonyl-piperazin-1-carbonyl; N-mono- or N,N-di-(C₁-C₇-alkyl)-amino-substituted or unsubstituted pyrrolidinyl-C₁-C₇-alkyl-carbonyl; cyano; C₁-C₇-alkenylene or -alkinylene; C₁-C₇-alkylsulfonyl (═C₁-C₇-alkane-sulfonyl); phenyl- or naphthylsulfonyl wherein phenyl or naphthyl is unsubstituted or substituted by one or more, especially one to three, moieties independently selected from the group consisting of C₁-C₇-alkyl, hydroxy, C₁-C₇-alkoxy and cyano; phenyl- or naphthyl-C₁-C₇-alkylsulfonyl; sulfamoyl; N-mono or N,N-di-[C₁-C₇-alkyl, phenyl-, naphthyl-, phenyl-C₁-C₇-alkyl-, pyrrolidinyl (especially pyrrolidino)-C₁-C₇-alkyl, piperidinyl (especially piperidino)-C₁-C₇-alkyl, piperazinyl (especially piperazino)-C₁-C₇-alkyl, N—C₁-C₇-alkylpiperazinyl (especially 4-C₁-C₇-alkylpiperazino)-C₁-C₇-alkyl, naphthyl-C₁-C₇-alkyl, phenyl which is unsubstituted or substituted by C₁-C₇-alkoxy, by halo, especially fluoro, by pyrrolidino, by piperidino, by piperazino, by hydroxyl-C₁-C₇-alkyl or by N-mono- or N,N-di-(C₁-C₇-alkyl)-C₁-C₇-alkyl; pyrrolidinyl (especially pyrrolidino), piperidinyl (especially piperidino), piperazinyl (especially piperazino), pyridinyl, pyrimidinyl, pyrazinyl, pyridazinyl, oxazolyl and/or thiazolyl]-aminosulfonyl; pyrazolyl; pyrazolidinyl; pyrrolyl; pyridinyl that is unsubstituted or substituted by C₁-C₇-alkoxy, such as methoxy, and/or by halo-C₁-C₇-alkyl, such as trifluoromethyl; pyrrolidinyl, such as pyrrolidin-1-yl; oxo-pyrrolidinyl, such as 2-oxo-pyrrolidin-1-yl; piperidinyl; oxo-piperidinyl, such as 2-oxopiperidin-1-yl; morpholinyl, such as morpholino; thiomorpholinyl, such as thiomorpholino; S-oxo-thiomorpholinyl, such as S-oxo-thiomorpholino; S,S-dioxothiomorpholinyl, such as S,S-dioxo-thiomorpholino; piperazinyl; N—C₁-C₇-alkyl-piperazinyl; 4-(phenyl-C₁-C₇-alkyl)-piperazinyl; 4-(naphthyl-C₁-C₇-alkyl)-piperazinyl; 4-(C₁-C₇-alkoxycarbonyl)-piperazinyl; 4-(phenyl-C₁-C₇-alkoxycarbonyl)-piperazinyl; 4-(naphthyl-C₁-C₇-alkoxycarbonyl)-piperazinyl; oxazolyl; thiazolyl; triazolyl, e.g. 1,2,4-triazol-1-yl; carbamoyl-triazolyl, e.g. carbamoyl-1,2,4-triazol-1-yl, such as 3-carbamoyl-1,2,4-triazol-1-yl; pyrazolyl, such as pyrazol-1-yl; halo-C₁-C₇-alkyl-pyrazolyl, such as 3-trifluoromethyl-pyrazol-1-yl; halophenyl-pyrazolyl, such as 3-(halophenyl)-pyrazol-1-yl, e.g. 3-(4-chlorophenyl)-pyrazol-1-yl; pyrimidin-(2-, 4- or 5-)yl; benzimidazol (especially -1-)yl; (e.g. 5-)C₁-C₇-alkoxy-substituted benzimidazol (especially -1-)yl; pyrrolo-pyrimidinyl, especially pyrrolo[2,3-d]pyrimidin-(e.g. 1-)yl; C₁-C₇-alkyl-substituted pyrrolo-pyrimidinyl, e.g. 2-C₁-C₇-alkyl-pyrrolo[2,3-d]pyrimidin-(e.g. 1-)yl (meaning 2-C₁-C₇-alkyl-5,7-diazaindol-1-yl); 1H,4H,5H-trihydropyrazolo[2,3-c]piperidin-1-yl (meaning 5-aza-3,4,5,6-tetrahydroindazol-1-yl) which is unsubstituted or substituted by 1 or 2 substituents independently selected from C₁-C₇-alkyl (e.g. methyl, especially in 5-position) and halo-C₁-C₇-alkyl (e.g. trifluoromethyl, especially in 3-position); nitro and/or further from C₃-C₈-cycloalkyl, phenyl or naphthyl each of which is unsubstituted or substituted by one or more, e.g. up to 2, moieties independently selected from the group consisting of halo, C₁-C₇-alkoxy, C₁-C₇-alkanesulfonyl, nitro and cyano; tetrazolyl, e.g. tetrazol-5-yl; indol-(e.g. 5-)yl; indazolyl, e.g. indazol-5-yl; (e.g. 3-) C₁-C₇-alkyl-indazoyl-(e.g. 5-)yl; and pyrrolo-pyridinyl, e.g. pyrrolo[2,3-c]pyridine-1-yl (meaning 5-aza-indol-1-yl). Especially preferably unsubstituted or substituted aryl is phenyl or naphthyl, each of which is unsubstituted or substituted as just described, more preferably by one or more, e.g. up to three, substituents independently selected from those mentioned above.

Substituted phenyl or substituted naphthyl (especially as R²) is especially phenyl or naphthyl, especially phenyl, where phenyl or naphthyl is substituted by one or more, preferably 1 to 3, more preferably 1 or 2, substituents (especially in meta- and/or para-position) selected from the group of substituents mentioned for substituted aryl, especially from the group consisting of C₁-C₇-alkyl, phenyl that is unsubstituted or substituted by one to three moieties independently selected from hydroxy and C₁-C₇-alkoxy, such as methoxy, halo, especially fluoro, hydroxy, C₁-C₇-alkoxy (very preferred), especially methoxy, hydroxy-C₁-C₇-alkoxy, C₁-C₇-alkoxy-C₁-C₇-alkoxy, especially 2-methoxyethoxy, 2-ethoxyethoxy, 2- or 3-methoxypropoxy, 2- or 3-ethoxypropoxy or 2- or 3-propoxypropoxy, C₁-C₇-alkoxy-C₁-C₇-alkoxy-C₁-C₇-alkoxy, such as 2-(2-methoxyethoxy or 2-ethoxyethoxy)-ethoxy, amino-C₁-C₇-alkoxy, N-mono- or N,N-di-(C₁-C₇-alkyl)-amino-C₁-C₇-alkoxy, e.g. 2-dimethyl- or 2-diethyl-amino-ethoxy or 2- or 3-dimethyl- or 2- or 3-diethyl-amino-propoxy, C₁-C₇-alkoxycarbonylamino-C₁-C₇-alkoxy, C₆-C₁₄-arylcarbonylamino-C₂-C₇-alkoxy (C₆-C₁₄-aryl-C(═O)—NH—C₂-C₇-alkoxy or C₆-C₁₄-aroyl-NH—C₂-C₇-alkoxy) wherein C₆-C₁₄-aryl is unsubstituted or substituted by one or more, especially up to three, substituents independently selected from the group consisting of C₁-C₇-alkyl, especially methyl or ethyl, halo-C₁-C₇-alkyl, especially trifluoromethyl, hydroxy, C₁-C₇-alkoxy, especially methoxy, and halo, especially fluoro, pyrrolyl-C₁-C₇-alkoxy, pyrrolidinyl-C₁-C₇-alkoxy wherein pyrrolidinyl is unsubstituted or substituted by oxo, pyrrolidinyl-C₁-C₇-alkoxy, imidazolyl-C₁-C₇-alkoxy, imidazolidinyl-C₁-C₇-alkoxy wherein imidazolidinyl is unsubstituted or substituted by oxo, piperidinyl-C₁-C₇-alkoxy, e.g. piperidino-C₁-C₇-alkoxy, piperazinyl-C₁-C₇-alkoxy wherein piperazinyl is unsubstituted or substituted with C₁-C₇-alkyl, morpholinyl-C₁-C₇-alkoxy, e.g. morpholino-C₁-C₇-alkoxy, thiomorpholinyl-C₁-C₇-alkoxy, e.g. thiomorpholino-C₁-C₇-alkoxy, S-oxo-thiomorpholinyl-C₁-C₇-alkoxy, e.g. S-oxothiomorpholino-C₁-C₇-alkoxy, S,S-dioxothiomorpholinyl-C₁-C₇-alkoxy, e.g. S,S-dioxothiomorpholino-C₁-C₇-alkoxy, piperazinyl-C₁-C₇-alkoxy, e.g. piperazino-C₁-C₇-alkoxy, N′—C₁-C₇-alkyl-piperazino-C₁-C₇-alkoxy, C₃-C₈-cyloalkoxy, pyridincarbonylamino-C₁-C₇-alkoxy, C₆-C₁₄-arylaminocarbonylamino-C₂-C₇-alkoxy (C₆-C₁₄-aryl-NH—C(═O)—NH—C₂-C₇-alkoxy) wherein C₆-C₁₄-aryl is defined as above, preferably is phenyl or naphthyl, and is in each case unsubstituted or substituted by one or more, especially up to three, substituents independently selected from the group consisting of C₁-C₇-alkyl, especially methyl or ethyl, halo-C₁-C₇-alkyl, especially trifluoromethyl, hydroxy, C₁-C₇-alkoxy, especially methoxy, and halo, especially fluoro, pyridinylaminocarbonylamino-C₂-C₇-alkoxy, C₁-C₇-alkane-sulfonyl, e.g. methane- or ethanesulfonyl, C₃-C₈-cyloalkyl-sulfonyl, nitro and cyano.

Preferably, substituted phenyl or substituted naphthyl R² carries at least one substituent (especially as defined in the last paragraph) in p-position and a methoxy in meta-position.

Generally, in the case of R¹ substituents in substituted heterocyclyl R¹ can be in the ortho- or preferably the meta- or para-position in the case of six-membered cycles, or generally expressed in position 2 or preferably 3 or 4 relative to the atom binding to the rest of the molecule.

An N-oxide derivative or pharmaceutically acceptable salt of each of the compounds of the formula I is also within the scope of this invention. For example, a nitrogen ring atom of a nitrogen-containing heterocyclic (e.g. heteroaryl) can form an N-oxide in the presence of a suitable oxidizing agent, e.g. a peroxide, such as m-chloro-perbenzoic acid or hydrogen peroxide.

Wherever a compound or compounds of the formula I are mentioned, this is further also intended to include (as alternative to the compound or in addition) one or more N-oxides of such compounds, also where not stated explicitly.

The term “an N-oxide thereof, a solvate thereof and/or a pharmaceutically acceptable salt thereof” especially means that a compound of the formula I may be present as such or in mixture with its N-oxide or as essentially pure N-oxide, as a solvate of the compound or the N-oxide, or as a salt of the compound of the formula I or an N-oxide thereof, or as a solvate of such salt and/or N-oxide, either each of these forms in essentially pure form or as a mixture with one or more of the other forms.

Compounds of the formula I can also be modified by appending appropriate functionalities to enhance selective biological properties. Modifications of this kind are known in the art and include those that increase penetration into a given biological system (e.g. blood, lymphatic system, central nervous system, testis), increase bioavailability, increase solubility to allow parenteral administration (e.g. injection, infusion), alter metabolism and/or alter the rate of secretion. Examples of this type of modifications include but are not limited to esterification, e.g. with polyethylene glycols, derivatisation with pivaloyloxy or fatty acid substituents, conversion to carbamates, hydroxylation of aromatic rings and heteroatom substitution in aromatic rings. Wherever compounds of the formula I, N-oxides, solvates and/or (especially pharmaceutically acceptable) salts thereof are mentioned, this comprises such modified formulae, while preferably the molecules of the formula I, N-oxides, solvates and/or (especially pharmaceutically acceptable) salts thereof as such are meant.

In view of the close relationship between the novel compounds of the formula I in free form and those in the form of their salts, including those salts that can be used as intermediates, for example in the purification or identification of the novel compounds, any reference to a compound or compounds of the formula I hereinbefore and hereinafter is to be understood as referring also to one or more salts, as appropriate and expedient, as well as to one or more solvates, e.g. hydrates.

Solvate means a (at least partially) crystalline compound of the formula I or a salt thereof in crystalline form with solvent molecules included in the crystal structure—the term solvate here includes hydrates (crystals including water molecules) and/or any other (preferably pharmaceutically acceptable) solvates with one or more other solvents.

Salts are formed, for example, as acid addition salts, preferably with organic or inorganic acids, from compounds of formula I with a basic nitrogen atom, and are especially pharmaceutically acceptable salts. Suitable inorganic acids are, for example, halogen acids, such as hydrochloric acid, sulfuric acid, or phosphoric acid. Suitable organic acids are, for example, carboxylic, phosphonic, sulfonic or sulfamic acids, for example acetic acid, propionic acid, octanoic acid, decanoic acid, dodecanoic acid, glycolic acid, lactic acid, fumaric acid, succinic acid, malonic acid, adipic acid, pimelic acid, suberic acid, azelaic acid, malic acid, tartaric acid, citric acid, amino acids, such as glutamic acid or aspartic acid, maleic acid, hydroxymaleic acid, methylmaleic acid, cyclohexanecarboxylic acid, adamantanecarboxylic acid, benzoic acid, salicylic acid, 4-aminosalicylic acid, phthalic acid, phenylacetic acid, mandelic acid, cinnamic acid, methane- or ethane-sulfonic acid, 2-hydroxyethanesulfonic acid, ethane-1,2-disulfonic acid, benzenesulfonic acid, 4-toluenesulfonic acid, 2-naphthalenesulfonic acid, 1,5-naphthalene-disulfonic acid, 2- or 3-methylbenzenesulfonic acid, methylsulfuric acid, ethylsulfuric acid, dodecylsulfuric acid, N-cyclohexylsulfamic acid, N-methyl-, N-ethyl- or N-propyl-sulfamic acid, or other organic protonic acids, such as ascorbic acid.

For isolation or purification purposes it is also possible to use pharmaceutically unacceptable salts, for example picrates or perchlorates. For therapeutic use, only pharmaceutically acceptable salts or free compounds are employed (where applicable in the form of pharmaceutical preparations), and these are therefore preferred.

Preferred is a compound of the formula I, wherein

R¹ is heterocyclyl that is unsaturated, partially saturated or saturated, preferably unsaturated, and has 4 to 10 ring atoms of which 1 to 3 are nitrogen, especially pyridinyl, more especially pyridin-2-yl or pyridin-3-yl, pyrimidinyl, pyrazinyl, especially pyrazin-2-yl, pyridazinyl, pyrazolyl, especially pyrazol-3-yl, or imidazolyl, each of which (including heterocyclyl) is unsubstituted or substituted by one or more, preferably one or two, substituents independently selected from the group consisting of unsubstituted or substituted alkyl as defined above, especially C₁-C₇-alkyl that is unsubstituted or substituted by hydroxyl, by halo, e.g. in trifluoromethyl, or by cyano-C₁-C₇-alkyl, halo, hydroxyl, alkyloxy, especially C₁-C₇-alkoxy, more especially methoxy, amino, mono- or disubstituted amino, preferably N-mono- or N,N-di-(C₁-C₇-alkyl and/or C₃-C₈-cyloalkyl)-amino, especially N-methylamino, C₁-C₇-alkanoylamino, C₁-C₇-alkoxycarbonyl-amino, phenyl- or naphthyl-C₁-C₇-alkoxycarbonylamino, carbamoyl, mono- or disubstituted carbamoyl, preferably N-mono- or N,N-di-(C₁-C₇-alkyl and/or C₃-C₈-cycloalkyl)-carbamoyl, heterocyclyl (especially pyrazolyl, such as pyrazolo, pyrrolidinyl, such as pyrrolidin-1-yl, pyridinyl, such as pyridin-(2-, 3- or 4-)yl, piperidinyl, such as piperidin-1-yl, oxopiperidinyl, such as 2-oxopiperidin-1-yl, piperazinyl, such as piperazin-1-yl, triazolyl, such as 1,2,4-triazol-1-yl, morpholinyl, such as morpholino, thiomorpholinyl, such as thiomorpholino, S-oxothiomorpholinyl, such as S-oxothiomorpholino, benzimidazol (especially -1-)yl, pyrrolo-pyrimidinyl, especially pyrrolo[2,3-d]pyrimidin-(e.g. 1-)yl, or 1H,4H,5H-trihydropyrazolo[2,3-c]piperidin-1-yl (meaning 5-aza-3,4,5,6-tetrahydroindazol-1-yl)) bound via a ring carbon atom or preferably a ring nitrogen and that is unsubstituted or substituted by one or more, especially up to three, substituents independently selected from C₁-C₇-alkyl, halo-C₁-C₇-alkyl, such as trifluoromethyl, halophenyl, such as 4-chlorophenyl, hydroxy, C₁-C₇-alkoxy, halo, C₁-C₇-alkoxycarbonyl, carbamoyl, phenylsulfonyl wherein phenyl is unsubstituted or substituted by one or more, preferably up to three, substituents independently selected from C₁-C₇-alkyl, hydroxy, C₁-C₇-alkoxy, halo, nitro and cyano, heterocyclylcarbonyl (=heterocyclyl-C(═O)—) where heterocyclyl is bound via a ring nitrogen to the carbonyl, especially piperidinocarbonyl, morpholino-carbonyl, thiomorpholino-carbonyl or S-oxo- or S,S-dioxothiomorpholinocarbonyl, C₁-C₇-alkanesulfonyl, such as methanesulfonyl, sulfamoyl, N-mono- or N,N-disubstituted sulfamoyl, preferably N-mono- or N,N-di-(C₁-C₇-alkyl)-sulfamoyl, cyano and nitro, and/or, in a broader aspect of the invention, further from unsubstituted or substituted aryl, from unsubstituted or substituted cycloalkyl and from unsubstituted or substituted heterocyclyl, and

R² is phenyl or naphthyl, especially phenyl, where phenyl or naphthyl is substituted by one or more, preferably 1 to 3, more preferably 1 or 2, substituents (especially in meta- and/or para-position) selected from the group consisting of C₁-C₇-alkyl, phenyl that is unsubstituted or substituted by one to three moieties independently selected from hydroxy and C₁-C₇-alkoxy, such as methoxy, halo, especially fluoro, hydroxy, C₁-C₇-alkoxy (very preferred), especially methoxy, hydroxy-C₁-C₇-alkoxy, C₁-C₇-alkoxy-C₁-C₇-alkoxy, especially 2-methoxyethoxy, 2-ethoxyethoxy, 2- or 3-methoxypropoxy, 2- or 3-ethoxypropoxy or 2- or 3-propoxypropoxy, C₁-C₇-alkoxy-C₁-C₇-alkoxy-C₁-C₇-alkoxy, such as 2-(2-methoxyethoxy or 2-ethoxyethoxy)-ethoxy, amino-C₁-C₇-alkoxy, N-mono- or N,N-di-(C₁-C₇-alkyl, phenyl- or naphthyl-C₁-C₇-alkyl and/or C₁-C₇-alkanoyl)-amino-C₁-C₇-alkoxy, e.g. 2-dimethyl- or 2-diethyl-amino-ethoxy or 2- or 3-dimethyl- or 2- or 3-diethyl-amino-propoxy, C₁-C₇-alkoxycarbonylamino-C₁-C₇-alkoxy, C₆-C₁₄-arylcarbonylamino-C₂-C₇-alkoxy (C₆-C₁₄-aryl-C(═O)—NH—C₂-C₇-alkoxy or C₆-C₁₄-aroyl-NH—C₂-C₇-alkoxy) wherein C₆-C₁₄-aryl is unsubstituted or substituted by one or more, especially up to three, substituents independently selected from the group consisting of C₁-C₇-alkyl, especially methyl or ethyl, halo-C₁-C₇-alkyl, especially trifluoromethyl, hydroxy, C₁-C₇-alkoxy, especially methoxy, halo, especially fluoro, and cyano, pyrrolyl-C₁-C₇-alkoxy, pyrrolidinyl-C₁-C₇-alkoxy wherein pyrrolidinyl is unsubstituted or substituted by oxo, pyrazolyl-C₁-C₇-alkoxy, pyrazolidinyl-C₁-C₇-alkoxy, imidazolyl-C₁-C₇-alkoxy, imidazolidinyl-C₁-C₇-alkoxy wherein imidazolidinyl is unsubstituted or substituted by oxo, piperidinyl-C₁-C₇-alkoxy, e.g. piperidino-C₁-C₇-alkoxy, piperazinyl-C₁-C₇-alkoxy wherein piperazinyl is unsubstituted or substituted with C₁-C₇-alkyl, morpholinyl-C₁-C₇-alkoxy, e.g. morpholino-C₁-C₇-alkoxy, thiomorpholinyl-C₁-C₇-alkoxy, e.g. thiomorpholino-C₁-C₇-alkoxy, S-oxo-thiomorpholinyl-C₁-C₇-alkoxy, e.g. S-oxothiomorpholino-C₁-C₇-alkoxy, S,S-dioxothiomorpholinyl-C₁-C₇-alkoxy, e.g. S,S-dioxothiomorpholino-C₁-C₇-alkoxy, C₃-C₈-cycloalkoxy, heterocyclylcarbonylamino-C₁-C₇-alkoxy wherein heterocyclyl has 3 to 10 ring atoms and has one or more hetero ring atoms selected from O, S and N, especially N, such as pyridincarbonylamino-C₂-C₇-alkoxy, C₆-C₁₄-arylaminocarbonylamino-C₂-C₇-alkoxy (C₆-C₁₄-aryl-NH—C(═O)—NH—C₂-C₇-alkoxy) wherein C₆-C₁₄-aryl is defined as above, preferably is phenyl or naphthyl, and is in each case unsubstituted or substituted by one or more, especially up to three, substituents independently selected from the group consisting of C₁-C₇-alkyl, especially methyl or ethyl, halo-C₁-C₇-alkyl, especially trifluoromethyl, hydroxy, C₁-C₇-alkoxy, especially methoxy, halo, especially fluoro, and cyano, heterocyclylaminocarbonylamino-C₁-C₇-alkoxy wherein heterocyclyl has 3 to 10 ring atoms and has one or more hetero ring atoms selected from O, S and N, especially N, such as pyridinylaminocarbonylamino-C₂-C₇-alkoxy, C₁-C₇-alkane-sulfonyl, e.g. methane- or ethanesulfonyl, C₃-C₃-cyloalkyl-sulfonyl, nitro and cyano;

preferably with the proviso that phenyl R² is substituted in meta position by C₁-C₇-alkoxy, especially methoxy, and in para-position by one more substituent independently selected from the group of substituents mentioned above for substituted phenyl R², more preferably of the substitutents C₁-C₇-alkoxy, especially methoxy, hydroxyl, C₁-C₇-alkoxy-C₁-C₇-alkoxy, especially 2-methoxyethoxy, 2-ethoxyethoxy, 2- or 3-methoxypropoxy, 2- or 3-ethoxypropoxy or 2- or 3-propoxypropoxy, C₁-C₇-alkoxy-C₁-C₇-alkoxy-C₁-C₇-alkoxy, such as 2-(2-methoxyethoxy or 2-ethoxyethoxy)-ethoxy, amino-C₁-C₇-alkoxy, N-mono- or N,N-di-(C₁-C₇-alkyl)-amino-C₁-C₇-alkoxy, e.g. 2-dimethyl- or 2-diethyl-amino-ethoxy or 2- or 3-dimethyl- or 2- or 3-diethyl-amino-propoxy, pyrrolidinyl-C₁-C₇-alkoxy, oxopyrrolidinyl-C₁-C₇-alkoxy, imidazolidinyl-C₁-C₇-alkoxy, piperidinyl-C₁-C₇-alkoxy, e.g. piperidino-C₁-C₇-alkoxy, piperazinyl-C₁-C₇-alkoxy, N—C₁-C₇-alkylpiperazinyl-C₁-C₇-alkoxy, morpholinyl-C₁-C₇-alkoxy, e.g. morpholino-C₁-C₇-alkoxy, thiomorpholinyl-C₁-C₇-alkoxy, e.g. thiomorpholino-C₁-C₇-alkoxy, S-oxo-thiomorpholinyl-C₁-C₇-alkoxy, e.g. S-oxothiomorpholino-C₁-C₇-alkoxy, S,S-dioxothiomorpholinyl-C₁-C₇-alkoxy, e.g. S,S-dioxothiomorpholino-C₁-C₇-alkoxy, C₃-C₈-cycloalkoxy, C₆-C₁₄-arylcarbonylamino-C₁-C₇-alkoxy (C₆-C₁₄-aryl-C(═O)—NH—C₂-C₇-alkoxy or C₆-C₁₄-aroyl-NH—C₂-C₇-alkoxy) wherein C₆-C₁₄-aryl is unsubstituted or substituted by one or more, especially up to three, substituents independently selected from the group consisting of C₁-C₇-alkyl, especially methyl or ethyl, halo-C₁-C₇-alkyl, especially trifluoromethyl, hydroxy, C₁-C₇-alkoxy, especially methoxy, and halo, especially fluoro, pyridincarbonylamino-C₂-C₇-alkoxy, C₆-C₁₄-arylaminocarbonylamino-C₂-C₇-alkoxy (C₆-C₁₄-aryl-NH—C(═O)—NH—C₂-C₇-alkoxy) wherein C₆-C₁₄-aryl is unsubstituted or substituted by one or more, especially up to three, substituents independently selected from the group consisting of C₁-C₇-alkyl, especially methyl or ethyl, halo-C₁-C₇-alkyl, especially trifluoromethyl, hydroxy, C₁-C₇-alkoxy, especially methoxy, and halo, especially fluoro, pyridinylaminocarbonylamino-C₂-C₇-alkoxy, C₁-C₇-alkane-sulfonyl, e.g. methane- or ethanesulfonyl, or C₃-C₈-cyloalkyl-sulfonyl; where in one more preferred embodiment R² is 3,4-dimethoxyphenyl;

or an N-oxide thereof, a solvate and/or a (preferably pharmaceutically acceptable) salt thereof.

Highly preferably, the invention relates to a compound of the formula I wherein

R¹ is pyridinyl, especially pyridin-2-yl or pyridin-3-yl, pyrimidinyl, pyrazinyl, especially pyrazin-2-yl, pyrazolyl, especially pyrazol-3-yl, or imidazolyl, each of which is unsubstituted or substituted by one or more, preferably one or two, substituents independently selected from the group consisting of C₁-C₇-alkyl, such as methyl, of halo-C₁-C₇-alkyl, such as trifluoro-methyl, of hydroxy, of C₁-C₇-alkoxy, such as methoxy or ethoxy, of halo, especially fluoro, chloro or bromo, of amino, of C₁-C₇-alkoxycarbonylamino, such as tert-butoxycarbonylamino, of pyridinyl (especially pyridin-2-yl) that is unsubstituted or substituted by one or more, preferably one or two, moieties independently selected from the group consisting of C₁-C₇-alkyl, such as methyl, hydroxy, C₁-C₇-alkoxy, such as methoxy or ethoxy, halo, e.g. fluoro, chloro or bromo, amino, C₁-C₇-alkoxycarbonylamino, such as tert-butoxycarbonylamino, and cyano, of piperidinyl, especially piperidino or piperidin-4-yl, of 1-(C₁-C₇-alkoxy)-piperidin-4-yl, of piperazino, of 4-(C₁-C₇-alkoxycarbonyl)-piperazino, of morpholino, of thiomorpholino, of S-oxo or S,S-dioxothiomorpholino, of (unsubstituted or cyano- and/or hydroxy-substituted-phenyl)-sulfonyl and of cyano, preferably pyridin-3-yl, 3-methyl-pyridin-2-yl, 6-hydroxypyridin-3-yl, 6-ethoxy-pyridin-3-yl, 6-fluoro-pyridin-3-yl, 6-chloro-pyridin-3-yl, 6-amino-pyridin-3-yl, 5-cyano-pyridin-3-yl, 6-cyano-pyridin-3-yl, 6-amino-5-trifluoromethyl-pyridin-3-yl, 6-tert-butoxycarbonylamino-5-trifluoromethyl-pyridin-3-yl, 6-(piperazin-1-yl)-pyridin-3-yl, 6-(4-tert-butoxycarbonyl-piperazin-1-yl)-pyridin-3-yl, 6-morpholino-pyridin-3-yl, 6-(6-cyanopyridin-3-yl)-pyridin-3-yl; pyrazol-3-yl, 1-(4-hydroxyphenylsulfonyl)-pyrazol-2-yl, 1-(3- or 4-cyanophenylsulfonyl)-pyrazol-2-yl or pyrazin-2-yl, and

R² is phenyl or napthyl, especially phenyl, each of which is unsubstituted or substituted by one or more, especially one or two, substitutents selected from the group consisting of C₁-C₇-alkyl, phenyl that is unsubstituted or substituted by one to three moieties independently selected from hydroxy and C₁-C₇-alkoxy, such as methoxy, C₁-C₇-alkoxy, especially methoxy or ethoxy, hydroxy-C₂-C₇-alkoxy, especially 2-hydroxyethoxy or 3-hydroxypropoxy, C₁-C₇-alkoxy-C₂-C₇-alkoxy, (C₁-C₇-alkoxy-C₂-C₇-alkoxy)-C₂-C₇-alkoxy, especially 2-(2-methoxyethoxy)-ethoxy, amino-C₁-C₇-alkoxy, N-mono- or N,N-di-(C₁-C₇-alkyl)amino-C₁-C₇-alkoxy, especially 2-diethylamino-ethoxy or 3-diethylamino-propoxy, C₁-C₇-alkoxycarbonylamino-C₂-C₇-alkoxy, especially 2-tert-butoxycarbonylamino-ethoxy or 3-tert-butoxycarbonylamino-propoxy, C₁-C₇-alkanoylamino-C₁-C₇-alkoxy, C₆-C₁₄-arylcarbonylamino-C₁-C₇-alkoxy (C₆-C₁₄-aryl-C(═O)—NH—C₂-C₇-alkoxy or C₆-C₁₄-aroyl-NH—C₂-C₇-alkoxy) wherein C₆-C₁₄-aryl (which is preferably phenyl or naphthyl) is unsubstituted or substituted by one or more, especially up to three, substituents independently selected from the group consisting of C₁-C₇-alkyl, especially methyl or ethyl, halo-C₁-C₇-alkyl, especially trifluoromethyl, hydroxy, C₁-C₇-alkoxy, especially methoxy, halo, especially fluoro, and cyano, especially 2-benzoylamino-ethoxy, 3-benzoylaminopropoxy, 2-(3-trifluoromethylbenzoylamino)-ethoxy, 3-(3-trifluoromethylbenzoylamino)-propoxy, 2-(3-methoxybenzoyl, 3,4-dimethoxybenzoylamino, 2,3,4-trimethoxybenzoylamino or 3,4,5-trimethoxybenzoylamino)-ethoxy, 3-(3-methoxybenzoyl, 3,4-dimethoxybenzoylamino, 2,3,4-trimethoxybenzoylamino or 3,4,5-trimethoxybenzoylamino)-propoxy, 2-(3,4-difluorobenzoylamino)-ethoxy, 3-(3,4-difluorobenzoylamino)-propoxy, pyridincarbonylamino-C₂-C₇-alkoxy, especially 2-(pyridin-4-carbonylamino)-ethoxy, 2-(pyridin-3-carbonylamino)-ethoxy, 3-(pyridin-4-carbonylamino)-propoxy or 3-(pyridin-3-carbonylamino)-propoxy, C₁-C₇-alkylaminocarbonylamino-C₁-C₇-alkoxy, such as 2-tert-butylaminocarbonylamino-ethoxy or 3-tert-butylaminocarbonylamino-propoxy, C₆-C₁₄-arylaminocarbonylamino-C₂-C₇-alkoxy (C₆-C₁₄-aryl-NH—C(═O)—NH—C₂-C₇-alkoxy) wherein C₆-C₁₄-aryl (which is preferably phenyl or naphthyl) is unsubstituted or substituted by one or more, especially up to three, substituents independently selected from the group consisting of C₁-C₇-alkyl, especially methyl or ethyl, halo-C₁-C₇-alkyl, especially trifluoromethyl, hydroxy, C₁-C₇-alkoxy, especially methoxy, halo, especially fluoro, and cyano, especially 2-phenylaminocarbonyl-amino-ethoxy, 3-phenylaminocarbonylamino-propoxy, 2-(3-trifluoromethyl, 3-methoxyphenyl, 3,4-dimethoxyphenyl, 2,3,4-trimethoxyphenyl, 3,4,5-trimethoxyphenyl or 3,4-difluorophenyl)-aminocarbonylamino-ethoxy, 3-(3-trifluoromethyl, 3-methoxyphenyl, 3,4-dimethoxyphenyl, 2,3,4-trimethoxyphenyl, 3,4,5-trimethoxyphenyl or 3,4-difluorophenyl)-aminocarbonylaminopropoxy, pyridinylaminocarbonylamino-C₂-C₇-alkoxy, especially pyridin-3- or pyridin-4-ylaminocarbonyl-C₂-C₇-alkoxy, such as 2-[pyridin-3- or pyridin-4-yl]aminocarbonylamino)-ethoxy or 3-[pyridin-3- or pyridin-4-yl]-aminocarbonylamino)-propoxy, pyrrolyl-C₁-C₇-alkoxy, pyrrolidinyl-C₁-C₇-alkoxy wherein pyrrolidinyl is unsubstituted or substituted by oxo, especially 2-(pyrrolidin-1-yl or 2-oxopyrrolidin-1-yl)-ethoxy or 3-(pyrrolidin-1-yl or 2-oxopyrrolidin-1-yl)-propoxy, imidazolyl-C₁-C₇-alkoxy, such as 2-imidazol-1-yl-ethoxy or 3-imidazol-1-yl-propoxy, imidazolidinyl-C₁-C₇-alkoxy wherein imidazolidinyl is unsubstituted or substituted by oxo, morpholinyl-C₁-C₇-alkoxy, especially 2-morpholino-ethoxy or 3-morpholino-propoxy, thiomorpholinyl-C₁-C₇-alkoxy, especially 2-thiomorpholino-ethoxy or 3-thiomorpholino-propoxy, S-oxothiomorpholinyl, S,S-dioxothiomorpholinyl, piperidinyl-C₁-C₇-alkoxy wherein piperidinyl is unsubstituted or substituted with C₁-C₇-alkyl, piperazinyl-C₁-C₇-alkoxy wherein piperazinyl is unsubstituted or substituted with C₁-C₇-alkyl, especially 2-(4-methyl-piperazin-1-yl)-ethoxy or 3-(4-methyl-piperazin-1-yl)-propoxy, halo, especially fluoro, C₁-C₇-alkylsulfonyl (C₁-C₇-alkyl-S(═O)₂—), especially methanesulfonyl (H₃C—S(═O)₂)—) or ethanesulfonyl (H₃C—CH₂—S(═O)₂—), nitro and cyano; where R² is more preferably 4′-methoxy-biphenyl-4-yl, 3,4-dimethoxyphenyl, 4-(2-hydroxyethyl or 3-hydroxypropyl)-3-methoxy-phenyl, 4-ethoxy-3-methoxyphenyl, 4-[(2-methoxyethoxy)-ethoxy]-3-methoxy-phenyl, 4-(2-amino-ethoxy)-3-methoxy-phenyl, 4-(2-(tert-butoxycarbonylamino)-ethoxy-3-methoxy-phenyl, 4-(3-amino-propoxy)-3-methoxyphenyl, 4-(3-(tert-butoxycarbonylamino)-propoxy-3-methoxy-phenyl, 4-(2-(diethylamino)-ethoxy)-3-methoxy-phenyl, 4-(3-(diethylamino)-propoxy)-3-methoxy-phenyl, 4-(2-pyrrolidinoethoxy)-3-methoxy-phenyl, 4-(3-pyrrolidino-propoxy)-3-methoxy-phenyl, 4-(2-(2-oxopyrrolidino)-ethoxy)-3-methoxy-phenyl, 4-(3-(2-oxopyrrolidino)-propoxy)-3-methoxy-phenyl, 4-(2-imidazolidin-1-yl-ethoxy)-3-methoxy-phenyl, 4-(3-imidazolidin-1-yl-propoxy)-3-methoxyphenyl, 4-(2-morpholino-ethoxy)-3-methoxy-phenyl, 4-(3-morpholino-propoxy)-3-methoxyphenyl, 4-(2-thiomorpholino-ethoxy)-3-methoxy-phenyl, 4-(3-thiomorpholino-propoxy)-3-methoxy-phenyl, 4-[2-(4-methylpiperazino)-ethoxy]-3-methoxy-phenyl, 4-[3-(4-methylpiperazino)-propoxy]-3-methoxy-phenyl, 4-(2-benzoylamino-ethoxy)-3-methoxy-phenyl, 4-(3-benzoylamino-propoxy)-3-methoxy-phenyl, 4-[2-((3-trifluoromethylbenzoyl, 3-methoxybenzoyl, 3,4-dimethoxybenzoyl, 2,3,4-trimethoxybenzoyl, 3,4,5-trimethoxybenzoyl or 3,4-difluorobenzoyl)-amino)-ethoxy]-3-methoxy-phenyl, 4-[3-((3-trifluoromethylbenzoyl, 3-methoxybenzoyl, 3,4-dimethoxybenzoyl, 2,3,4-trimethoxybenzoyl, 3,4,5-trimethoxybenzoyl or 3,4-difluorobenzoyl)-amino)-propoxy]-3-methoxy-phenyl, 4-[2-(pyridin-4-carbonylamino)-ethoxy]-3-methoxy-phenyl, 4-[3-(pyridin-4-carbonylamino)-propoxy]-3-methoxy-phenyl, 4-[2-(pyridin-3-carbonylamino)-ethoxy]-3-methoxy-phenyl, 4-(3-[pyridin-3-carbonylamino)-propoxy]-3-methoxyphenyl, 4-(2-phenylaminocarbonylamino-ethoxy)-3-methoxy-phenyl, 4-(3-phenylaminocarbonylamino-propoxy)-3-methoxy-phenyl, 4-[2-((3-trifluoromethylphenyl-aminocarbonyl, 3-methoxyphenyl-aminocarbonyl, 3,4-dimethoxyphenyl-aminocarbonyl, 2,3,4-trimethoxyphenyl-aminocarbonyl, 3,4,5-trimethoxyphenyl-aminocarbonyl or 3,4-difluorophenyl-aminocarbonyl)-amino)-ethoxy]-3-methoxy-phenyl, 4-[3-((3-trifluoromethylphenyl-aminocarbonyl, 3-methoxy-phenyl-aminocarbonyl, 3,4-dimethoxyphenyl-aminocarbonyl, 2,3,4-trimethoxyphenyl-aminocarbonyl, 3,4,5-trimethoxyphenyl-aminocarbonyl or 3,4-difluorophenyl-aminocarbonyl)-amino)-propoxy]-3-methoxy-phenyl, 4-[2-(pyridin-4-ylamino-carbonylamino)-ethoxy]-3-methoxy-phenyl, 4-[3-(pyridin-4-ylcarbonylamino)-propoxy]-3-methoxy-phenyl, 4-[2-(pyridin-3-ylaminocarbonylamino)-ethoxy]-3-methoxy-phenyl, 4-[3-(pyridin-3-ylaminocarbonylamino)-propoxy)-3-methoxy-phenyl or 4-methanesulfonyl-phenyl;

or an N-oxide thereof, a solvate and/or a (preferably pharmaceutically acceptable) salt thereof.

Very preferred are also embodiments of the invention represented in the claims which are therefore incorporated by reference herein.

Any formula given herein is intended to represent compounds having structures depicted by the structural formula as well as certain variations or forms. In particular, compounds of any formula given herein may have asymmetric centers and therefore exist in different enantiomeric forms. If at least one asymmetrical carbon atom is present in a compound of the formula I, such a compound may exist in optically active form or in the form of a mixture of optical isomers, e.g. in the form of a racemic mixture. All optical isomers and their mixtures, including the racemic mixtures, are part of the present invention. Thus, any given formula given herein is intended to represent a racemate, one or more enantiomeric forms, one or more diastereomeric forms, one or more atropisomeric forms, and mixtures thereof. Furthermore, certain structures may exist as geometric isomers (i.e. cis and trans isomers), as tautomers, or as atropisomers.

Any formula given herein is intended to represent hydrates, solvates, and polymorphs of such compounds, and mixtures thereof.

Any formula given herein is also intended to represent unlabeled forms as well as isotopically labeled forms of the compounds. Isotopically labeled compounds have structures depicted by the formulas given herein except that one or more atoms are replaced by an atom having a selected atomic mass or mass number. Examples of isotopes that can be incorporated into compounds of the invention include isotopes of hydrogen, carbon, nitrogen, oxygen, phosphorous, fluorine, and chlorine, such as ²H, ³H, ¹¹C, ¹³C, ¹⁴C, ¹⁵N, ¹⁸F ³¹P, ³²P, ³⁵S, ³⁶Cl, ¹²⁵I respectively. Various isotopically labeled compounds of the present invention, for example those into which radioactive isotopes such as ³H, ¹³C, and ¹⁴C are incorporated. Such isotopically labelled compounds are useful in metabolic studies (preferably with ¹⁴C), reaction kinetic studies (with, for example ²H or ³H), detection or imaging techniques [such as positron emission tomography (PET) or single-photon emission computed tomography (SPECT) including drug or substrate tissue distribution assays, or in radioactive treatment of patients. In particular, an ¹⁸F or labeled compound may be particularly preferred for PET or SPECT studies. Further, substitution with heavier isotopes such as deuterium (i.e., ²H) may afford certain therapeutic advantages resulting from greater metabolic stability, for example increased in vivo half-life or reduced dosage requirements. Isotopically labeled compounds of this invention and prodrugs thereof can generally be prepared by carrying out the procedures disclosed in the schemes or in the examples and preparations described below by substituting a readily available isotopically labeled reagent for a non-isotopically labeled reagent.

The invention relates especially to a compound of the formula I as mentioned below in the examples by their names, preferably the isomers shown as formulae, respectively, or a pharmaceutically acceptable salt thereof, or its USE according to the invention.

Quite unexpectedly, it has now been found that the compounds of formula I have advantageous pharmacological properties and inhibit the activity of the lipid kinases, such as the PI3-kinase and/or members of the PI3-kinase-related protein kinase family (also called PIKK and include DNA-PK, ATM, ATR, hSMG-1 and mTOR), such as the DNA protein-kinase, and may be used to treat disease or disorders which depend on the activity of said kinases.

The phosphatidylinositol-3′-OH kinase (PI3K) pathway is one of the central signaling pathways that exerts its effect on numerous cellular functions including cell cycle progression, proliferation, motility, metabolism and survival. An activation of receptor tyrosine kinases causes PI3K to phosphorylate phosphatidylinositol-(4,5)-diphosphate, resulting in membrane-bound phosphatidylinositol-(3,4,5)-triphosphate. The latter promotes the transfer of a variety of protein kinases from the cytoplasm to the plasma membrane by binding of phosphatidylinositol-(3,4,5)-triphosphate to the pleckstrin-homology (PH) domain of the kinase. Kinases that are key downstream targets of PI3K include phosphoinositide-dependent kinase 1 (PDK1) and AKT (also known as Protein Kinase B). Phosphorylation of such kinases then allows for the activation or deactivation of numerous other pathways, involving mediators such as GSK3, mTOR, PRAS40, FKHD, NF-κB, BAD, Caspase-9, and the like. An important negative feedback mechanism for the PI3K pathway is PTEN, a phosphatase that catalyses the dephosphorylation of phosphatidylinositol-(3,4,5)-triphosphate to phosphorylate phosphatidylinositol-(4,5)-diphosphate. In more than 60% of all solid tumors, PTEN is mutated into an inactive form, permitting a constitutive activation of the PI3K pathway. As most cancers are solid tumors, such an observation provides evidence that a targeting of PI3k itself or individual downstream kinases in the PI3K pathway provide a promising approach to mitigate or even abolish the dysregulation in many cancers and thus restore normal cell function and behaviour. This, however, does not exclude that other mechanisms may be responsible for the beneficial effects of PI3K activity modifying agents such as those in the present invention.

Having regard to their inhibitory effect on phosphatidylinositol 3-kinase enzymes, compounds of formula (I) in free or pharmaceutically acceptable salt form, are useful in the treatment of conditions which are mediated by the activation (including normal activity or especially over-activity) of one or more of the members of the PI3 kinase family, especially PI3 kinase enzyme, such as proliferative, inflammatory or allergic conditions, obstructive airways diseases and/or disorders commonly occurring in connection with transplantation.

“Treatment” in accordance with the invention may be therapeutic, e.g. symptomatic, and/or prophylactic. Preferred is the treatment of warm-blooded animals, especially humans.

Preferred is a compound of formula I for use or the use thereof in the treatment of a proliferative disease selected from a benign or malignant tumor, carcinoma of the brain, kidney, liver, adrenal gland, bladder, breast, stomach, gastric tumors, ovaries, colon, rectum, prostate, pancreas, lung, vagina or thyroid, sarcoma, glioblastomas, multiple myeloma or gastrointestinal cancer, especially colon carcinoma or colorectal adenoma or a tumor of the neck and head, an epidermal hyperproliferation, psoriasis, prostate hyperplasia, a neoplasia, a neoplasia of epithelial character, lymphomas, a mammary carcinoma or a leukemia. Other diseases include Cowden syndrome, Lhermitte-Dudos disease and Bannayan-Zonana syndrome, or diseases in which the PI3K/PKB pathway is aberrantly activated.

Compounds according to the invention are also of use in the treatment of inflammatory or obstructive airways (respiratory tract) diseases, resulting, for example, in reduction of tissue damage, airways inflammation, bronchial hyperreactivity, remodeling or disease progresssion. Inflammatory or obstructive airways diseases to which the present invention is applicable include asthma of whatever type or genesis including both intrinsic (non-allergic) asthma and extrinsic (allergic) asthma, e.g. mild asthma, moderate asthma, severe asthma, bronchitic asthma, exercise-induced asthma, occupational asthma and asthma induced following bacterial infection. Treatment of asthma is also to be understood as embracing treatment of subjects, e.g. of less than 4 or 5 years of age, exhibiting wheezing symptoms and diagnosed or diagnosable as “wheezy infants”, an established patient category of major medical concern and now often identified as incipient or early-phase asthmatics. (For convenience this particular asthmatic condition is referred to as “wheezy-infant syndrome”.)

Prophylactic efficacy in the treatment of asthma can be evidenced by reduced frequency or severity of symptomatic attack, e.g. of acute asthmatic or bronchoconstrictor attack, improvement in lung function or improved airways hyperreactivity. It may further be evidenced by reduced requirement for other, symptomatic therapy, i.e. therapy for or intended to restrict or abort symptomatic attack when it occurs, for example anti-inflammatory (e.g. corticosteroid) or bronchodilatory. Prophylactic benefit in asthma may in particular be apparent in subjects prone to “morning dipping”. “Morning dipping” is a recognised asthmatic syndrome, common to a substantial percentage of asthmatics and characterised by asthma attack, e.g. between the hours of about 4 to 6 am, i.e. at a time normally substantially distant form any previously administered symptomatic asthma therapy.

Compounds of the formula I can be of use for other inflammatory or obstructive airways diseases and conditions to which the present invention is applicable and include acute lung injury (ALI), adult/acute respiratory distress syndrome (ARDS), chronic obstructive pulmonary, airways or lung disease (COPD, COAD or COLD), including chronic bronchitis or dyspnea associated therewith, emphysema, as well as exacerbation of airways hyperreactivity consequent to other drug therapy, in particular other inhaled drug therapy.

The invention also relates to the treatment of bronchitis of whatever type or genesis including, e.g., acute, arachidic, catarrhal, croupus, chronic or phthinoid bronchitis. Further inflammatory or obstructive airways diseases to which the present invention is applicable include pneumoconiosis (an inflammatory, commonly occupational, disease of the lungs, frequently accompanied by airways obstruction, whether chronic or acute, and occasioned by repeated inhalation of dusts) of whatever type or genesis, including, for example, aluminosis, anthracosis, asbestosis, chalicosis, ptilosis, siderosis, silicosis, tabacosis and byssinosis. Having regard to their anti-inflammatory activity, in particular in relation to inhibition of eosinophil activation, compounds of the invention are also of use in the treatment of eosinophil related disorders, e.g. eosinophilia, in particular eosinophil related disorders of the airways (e.g. involving morbid eosinophilic infiltration of pulmonary tissues) including hypereosinophilia as it effects the airways and/or lungs as well as, for example, eosinophil-related disorders of the airways consequential or concomitant to Loffler's syndrome, eosinophilic pneumonia, parasitic (in particular metazoan) infestation (including tropical eosinophilia), bronchopulmonary aspergillosis, polyarteritis nodosa (including Churg-Strauss syndrome), eosinophilic granuloma and eosinophil-related disorders affecting the airways occasioned by drug-reaction.

Compounds of the invention are also of use in the treatment of inflammatory or allergic conditions of the skin, for example psoriasis, contact dermatitis, atopic dermatitis, alopecia greata, erythema multiforma, dermatitis herpetiformis, scleroderma, vitiligo, hypersensitivity angiitis, urticaria, bullous pemphigoid, lupus erythematosus, pemphigus, epidermolysis bullosa acquisita, and other inflammatory or allergic conditions of the skin.

Compounds of the invention may also be used for the treatment of other diseases or conditions, such as diseases or conditions having an inflammatory component, for example, treatment of diseases and conditions of the eye such as conjunctivitis, keratoconjunctivitis sicca, and vernal conjunctivitis, diseases affecting the nose including allergic rhinitis, and inflammatory disease in which autoimmune reactions are implicated or having an autoimmune component or aetiology, including autoimmune haematological disorders (e.g. haemolytic anaemia, aplastic anaemia, pure red cell anaemia and idiopathic thrombocytopenia), systemic lupus erythematosus, polychondritis, sclerodoma, Wegener granulamatosis, dermatomyositis, chronic active hepatitis, myasthenia gravis, Steven-Johnson syndrome, idiopathic sprue, autoimmune inflammatory bowel disease (e.g. ulcerative colitis and Crohn's disease), endocrine opthalmopathy, Grave's disease, sarcoidosis, alveolitis, chronic hypersensitivity pneumonitis, multiple sclerosis, primary billiary cirrhosis, uveitis (anterior and posterior), keratoconjunctivitis sicca and vernal keratoconjunctivitis, interstitial lung fibrosis, psoriatic arthritis and glomerulonephritis (with and without nephrotic syndrome, e.g. including idiopathic nephrotic syndrome or minimal change nephropathy).

Furthermore, the invention provides the use of a compound according to the definitions herein, an N-oxide, a pharmaceutically acceptable salt, and/or a hydrate or solvate thereof for the preparation of a medicament for the treatment of a proliferative disease, an inflammatory disease, an obstructive respiratory disease, or a disorder commonly occurring in connection with transplantation.

The invention especially relates to the use of a compound of the formula I (or a pharmaceutical formulation comprising a compound of the formula I) in the treatment of one or more of the diseases mentioned above and below where the disease(s) respond or responds (in a beneficial way, e.g. by partial or complete removal of one or more of its symptoms up to complete cure or remission) to an inhibition of one or more kinases of the PI3-kinase-related protein kinase family, most especially PI3 kinase (PI3K), especially where the kinase shows (in the context of other regulatory mechanisms) inadequately high or more preferably higher than normal (e.g. constitutive) activity.

Wherever the term “use” or “used” or especially “USE” is mentioned, this is intended to include a compound of the formula I (also the one excluded from the compound per se protection above and in the claims) for use in the prophylactic and/or therapeutic treatment of a disease of a warm-blooded animal, especially a human, preferably of one or more diseases mentioned above or below, a method of use or a method of treatment comprising administering a compound of the formula Ito a person in need of such treatment in an effective amount for the prophylactic and/or therapeutic treatment of a disease as mentioned above and below, the preparation or a method or preparation of a pharmaceutical formulation/preparation for use in the prophylactic and therapeutic treatment of a disease mentioned above and below, especially involving mixing a compound of the formula I (as therapeutically active ingredient) with at least one pharmaceutically acceptable carrier material, including making it ready for use in such treatment (e.g. adding an instruction insert (e.g. package leaflet or the like), formulation, appropriate preparation, adaptation for specific uses, customizing and the like), and the use of a compound of the formula I for such preparation, and/or all other prophylactic or therapeutic uses mentioned hereinbefore or below. All these aspects are embodiments of the present invention.

The efficacy of the compounds of formula I and salts thereof as PI3 kinase inhibitors can be demonstrated as follows:

The kinase reaction is performed in a final volume of 50 μL per well of a half area COSTAR, 96 well plate. The final concentrations of ATP and phosphatidyl inositol in the assay are 5 μM and 6 μg/mL respectively. The reaction is started by the addition of PI3 kinase, e.g. PI3 kinase.

p110β. The components of the assay are added per well, as follows:
10 μL test compound in 5% DMSO per well in columns 2-1.
Total activity is determined by addition 10 μL of 5% vol/vol DMSO in the first 4 wells of column 1 and the last 4 wells of column 12.
The background is determined by addition of 10 μM control compound to the last 4 wells of column 1 and the first 4 wells of column 12.
2 mL ‘Assay mix’ are prepared per plate:
   1.912 mL of HEPES assay buffer
   8.334 of 3 mM stock of ATP giving a final concentration of 5 μM per well
   1 μL of [³³P]ATP on the activity date giving 0.05 μCi per well
   30 μL of 1 mg/mL PI stock giving a final concentration of 6 μg/mL per well
   5 μL of 1 M stock MgCl₂ giving a final concentration of 1 mM per well
20 μL of the assay mix are added per well.
2 mL ‘Enzyme mix’ are prepared per plate (x* μL PI3 kinase p110β in 2 mL of kinase buffer). The ‘Enzyme mix’ is kept on ice during addition to the assay plates.
20 μl ‘Enzyme mix’ are added/well to start the reaction.
The plate is then incubated at room temperature for 90 minutes.
The reaction is terminated by the addition of 50 μL WGA-SPA bead (wheat germ agglutinin-coated Scintillation Proximity Assay beads) suspension per well.
The assay plate is sealed using TopSeal-S) heat seal for polystyrene microplates, PerkinElmer LAS (Deutschland) GmbH, Rodgau, Germany) and incubated at room temperature for at least 60 minutes.
The assay plate is then centrifuged at 1500 rpm for 2 minutes using the Jouan bench top centrifuge (Jouan Inc., Nantes, France).
The assay plate is counted using a Packard TopCount, each well being counted for 20 seconds.
   The volume of enzyme is dependent on the enzymatic activity of the batch in use.

In a more preferred assay, the kinase reaction is performed in a final volume of 10 μL per well of a low volume non binding CORNING, 384 well black plate (Cat. No. #3676). The final concentrations of ATP and phosphatidyl inositol (PI) in the assay are 1 μM and 10 μg/mL respectively. The reaction is started by the addition of ATP.

The components of the assay are added per well as follows:

50 nL test compounds in 90% DMSO per well, in columns 1-20, 8 concentrations (⅓ and 1/3.33 serial dilution step) in single.
Low control: 50 nL of 90% DMSO in half the wells of columns 23-24 (0.45% in final).
High control: 50 nL of reference compound (e.g. compound of Example 7 in WO 2006/122806, incorporated by reference herein that regard) in the other half of columns 23-24 (2.5 μM in final).
Standard: 50 nL of reference compound as just mentioned diluted as the test compounds in columns 21-22
20 mL ‘buffer’ are prepared per assay:
   200 μL of 1M TRIS HCl pH7.5 (10 mM in final)
   60 μL of 1M MgCl₂ (3 mM in final)
   500 μL of 2M NaCl (50 mM in final)
   100 μL of 10% CHAPS (0.05% in final)
   200 μL of 100 mM DTT (1 mM in final)
   18.94 mL of nanopure water
10 mL ‘PI’ are prepared per assay:
   200 μL of 1 mg/ml L-alpha-Phosphatidylinositol (Liver Bovine, Avanti Polar Lipids Cat. No. 840042C MW=909.12) prepared in 3% OctylGlucoside (10 μg/ml in final)
   9.8 mL of ‘buffer’
10 mL ‘ATP’ are prepared per assay:
   6.7 μL of 3 mM stock of ATP giving a final concentration of 1 μM per well
   10 mL of ‘buffer’
2.5 mL of each PI3K construct are prepared per assay in ‘PI’ with the following final concentration:
   10 nM PI3K alfa BV-1075
   25 nM beta BV-949
   10 nM delta BV-1060
   150 nM gamma BV-950
5 μL of ‘PI/PI3K’ are added per well.
5 μl ‘ATP’ are added per well to start the reaction.
The plates are then incubated at room temperature for 60 minutes (alfa, beta, delta) or 120 minutes (gamma).
The reaction is terminated by the addition of 10 μL Kinase-Glo (Promega Cat. No. #6714).
The assay plates are read after 10 minutes in Synergy 2 reader (BioTek, Vermont USA) with an integration time of 100 milliseconds and sensitivity set to 191.
Output: The High control is around 60′000 counts and the Low control is 30′000 or lower
This luminescence assay gives a useful Z′ ratio between 0.4 and 0.7
   The Z′ value is a universal measurement of the robustness of an assay. A Z′ between 0.5 and 1.0 is considered an excellent assay.

For this assay, the PI3K constructs mentioned are prepared as follows:

### Molecular Biology:

Two different constructs, BV 1052 and BV 1075, are used to generate the PI3 Kinase a proteins for compound screening.

PI3Kα BV-1052 p85(iSH2)-Gly linker-p110a(D20aa)-C-term His tag

PCR products for the inter SH2 domain (iSH2) of the p85 subunit and for the p110-a subunit (with a deletion of the first 20 amino acids) are generated and fused by overlapping PCR. The iSH2 PCR product is generated from first strand cDNA using initially primers

(SEQ ID NO: 1)

gwG130-p01 (5′-CGAGAATATGATAGATTATATGAAGAAT-3′)
and

(SEQ ID NO: 2)

gwG130-p02 (5′-TGGTTT-AATGCTGTTCATACGTTTGTCAAT-
3′).

Subsequently in a secondary PCR reaction, Gateway (Invitrogen AG, Basel, Switzerland) recombination AttB1 sites and linker sequences are added at the 5′ end and 3′ end of the p85 iSH2 fragment respectively, using primers

(SEQ ID NO: 3)

gwG130-p03 (5′-GGGACAAGTTTGTACAAAAAAGCAGGCTACGAAGG
AGATATACATAT-GCGAGAATATGATAGATTATATGAAGAAT-3′)
and

(SEQ ID NO: 4)

gwG152-p04 (5′-TACCATAATTCCACCACCACCACCGGAAATTCCCC
CTGGTTT-AATGCTGTTCATACGTTTGTCAAT-3′).

The p110-a fragment is also generated from first strand cDNA, initially using primers

(SEQ ID NO: 5)

gwG152-p01 (5′-CTAGTGGAATGTTTACTACCAAATGG-3′)
and

(SEQ ID NO: 6)

gwG152-p02 (5′-GTTCAATG-CATGCTGTTTAATTGTGT-3′).

In a subsequent PCR reaction, linker sequence and a Histidine tag are added at the 5′ end and 3′ end of the p110-a fragment respectively, using primers

(SEQ ID NO: 7)

gw152-p03 (5′-GGGGGAATTTCCGGTGGTGGTGGTGGAATTATGGTA
C-TAGTGGAATGTTTACTACC-AAATGGA-3′)
and

(SEQ ID NO: 8)

gwG152-p06 (5′-AGCTCCGTGATGGTGATGGTGATGTGCTCCGTTCA
ATG-CATGCTGTTTAATTGTGT-3′).

The p85-iSH2/p110-a fusion protein is assembled in a third PCR reaction by the overlapping linkers at the 3′ end of the iSH2 fragment and the 5′ end of the p110-a fragment, using the above mentioned gwG130-p03 primer and a primer containing an overlapping Histidine tag and the AttB2 recombination sequences

(SEQ ID NO: 9)

(5′-GGGACCACTTTGTACAAGAAAGCTGGGTTTAAGCTCCGTGATGGTG
ATGGTGAT-GTGCTCC-3′).

This final product is recombined in a (Invitrogen) OR reaction into the donor vector pDONR201 to generate the ORF318 entry clone. This clone is verified by sequencing and used in a Gateway LR reaction to transfer the insert into the Gateway adapted pBlueBac4.5 (Invitrogen) vector for generation of the baculovirus expression vector LR410.

PI3Kα BV-1075 p85(iSH2)-12 XGly linker-p110a(D20aa)-C-term His Tag

The construct for Baculovirus BV-1075 is generated by a three-part ligation comprised of a p85 fragment and a p110-a fragment cloned into vector pBlueBac4.5. The p85 fragment is derived from plasmid p1661-2 digested with Nhe/Spe. The p110-a fragment derived from LR410 (see above) as a SpeI/HindIII fragment. The cloning vector pBlueBac4.5 (Invitrogen) is digested with Nhe/HindIII. This results in the construct PED 153.8

The p85 component (iSH2) is generated by PCR using ORF 318 (described above) as a template and one forward primer

(SEQ ID NO: 10)

KAC1028 (5′-GCTAGCATGCGAGAATATGATAGATTATATGAAGAATA
TACC),
and two reverse primers

(SEQ ID NO: 11)

KAC1029 (5′-GCCTCCACCACCTCCGCCTGGTTTAATGCTGTTCATAC
GTTTGTC)
and

(SEQ ID NO: 12)

KAC1039 (5′-TACTAGTCCGCCTCCACCACCTCCGCCTCCACCACCT
CCGCC).

The two reverse primers overlap and incorporate the 12× Gly linker and the N-terminal sequence of the p110a gene to the SpeI site. The 12× Gly linker replaces the linker in the BV1052 construct. The PCR fragment is cloned into pCR2.1 TOPO (Invitrogen). Of the resulting clones, p1661-2 is determined to be correct. This plasmid is digested with Nhe and SpeI and the resulting fragment is gel-isolated and purified for sub-cloning.

The p110-a cloning fragment is generated by enzymatic digest of clone LR410 (see above) with Spe I and HindIII. The SpeI site is in the coding region of the p110a gene. The resulting fragment is gel-isolated and purified for sub-cloning.

The cloning vector, pBlueBac4.5 (Invitrogen) is prepared by enzymatic digestion with Nhe and HindIII. The cut vector is purified with Qiagen (Quiagen N.V, Venlo, Netherlands) column and then dephosphorylated with Calf Intestine alkaline phosphatase (CIP) (New England BioLabs, Ipswich, Mass.). After completion of the CIP reaction the cut vector is again column purified to generate the final vector. A 3 part ligation is performed using Roche Rapid ligase and the vendor specifications.

PI3Kβ BV-949 p85(iSH2)-Gly linker-p110b (Full-Length)-C-Term His Tag

PCR products for the inter SH2 domain (iSH2) of the p85 subunit and for the full-length p110-b subunit are generated and fused by overlapping PCR.

The iSH2 PCR product is generated from first strand cDNA initially using primers

(SEQ ID NO: 1)

gwG130-p01 (5′-CGAGAATATGATAGATTATATGAAGAAT-3′)
and

(SEQ ID NO: 2)

gwG130-p02 (5′-TGGTTT-AATGCTGTTCATACGTTTGTCAAT-
3′).

Subsequently, in a secondary PCR reaction Gateway (Invitrogen) recombination AttB1 sites and linker sequences are added at the 5′ end and 3′ end of the p85 iSH2 fragment respectively, using primers

(SEQ ID NO: 3)

gwG130-p03 (5′-GGGACAAGTTTGTACAAAAAAGCAGGCTACGAAGG
AGATA-TACATATGCGAGAATATGATAGATTATATGAAGAAT-3′)
and

(SEQ ID NO: 13)

gwG130-p05 (5′-ACTGAAGCATCCTCCTCCTCCTCCTCCTGGTTTAA
T-GCTGTTCATACGTTTGTC-3′).

The p110-b fragment is also generated from first strand cDNA initially using primers

(SEQ ID NO: 4)

gwG130-p04 (5′-ATTAAACCAGGAGGAGGAGGAGGAGGATGCTTCAG
TTTCATAATGCC-TCCTGCT-3′)

which contains linker sequences and the 5′ end of p110-b and

(SEQ ID NO: 14)

gwG130-p06 (5′-AGCTCCGTGATGGTGATGGTGATGTGCTCCAGATC
TGTAGTCTTT-CCGAACTGTGTG-3′)

which contains sequences of the 3′ end of p110-b fused to a Histidine tag. The p85-iSH2/p110-b fusion protein is assembled by an overlapping PCR a reaction of the linkers at the 3′ end of the iSH2 fragment and the 5′ end of the p110-b fragment, using the above mentioned gwG130-p03 primer and a primer containing an overlapping Histidine tag and the AttB2 recombination sequences (5′-GGGACCACTTTGTACAAGAAAGCTGGGTTT-AAGCTCCGTGATGGTGATGGTGATGTGCTCC-3′) (SEQ ID NO: 15).

This final product is recombined in a Gateway (Invitrogen) OR reaction into the donor vector pDONR201 to generate the ORF253 entry clone. This clone is verified by sequencing and used in a Gateway LR reaction to transfer the insert into the Gateway adapted pBlueBac4.5 (Invitrogen) vector for generation of the baculovirus expression vector LR280.

PI3Kδ BV-1060 p85(iSH2)-Gly Linker-p110d (Full-Length)-C-term His Tact

PCR products for the inter SH2 domain (iSH2) of the p85 subunit and for the full-length p110-d subunit are generated and fused by overlapping PCR.

The iSH2 PCR product is generated from first strand cDNA using initially primers

(SEQ ID NO: 1)

gwG130-p01 (5′-CGAGAATATGATAGATTATATGAAGAAT-3′)
and

(SEQ ID NO: 2)

gwG130-p02 (5′-TGGTTT-AATGCTGTTCATACGTTTGTCAAT-
3′).

Subsequently, in a secondary PCR reaction Gateway (Invitrogen) recombination AttB1 sites and linker sequences are added at the 5′ end and 3′ end of the p85 iSH2 fragment respectively, using primers

(SEQ ID NO: 3)

gwG130-p03 (5′-GGGACAAGTTTGTACAAAAAAGCAGGCTACGAAGG
AGATATACAT-ATGCGAGAATATGATAGATTATATGAAGAAT-3′)
and

(SEQ ID NO: 16)

gwG154-p04 (5′-TCCTCCTCCTCCTCCTCCTGGTTTAATGCTGTTCA
TACGTTTGTC-3′).

The p110-a fragment is also generated from first strand cDNA using initially primers

(SEQ ID NO: 17)

gwG154-p01 (5′-ATGCCCCCTGGGGTGGACTGCCCCAT-3′)
and

(SEQ ID NO: 18)

gwG154-p02 (5′-CTACTG-CCTGTTGTCTTTGGACACGT-3′).

In a subsequent PCR reaction linker sequences and a Histidine tag is added at the 5′ end and 3′ end of the p110-d fragment respectively, using primers

(SEQ ID NO: 19)

gw154-p03 (5′-ATTAAACCAGGAGGAGGAGGAGGAGGACCCCCTGGG
GTGGAC-TGCCCCATGGA-3′)
and

(SEQ ID NO: 20)

gwG154-p06 (5′-AGCTCCGTGATGGTGAT-GGTGATGTGCT-CCCTG
CCTGTTGTCTTTGGACACGTTGT-3′).

The p85-iSH2/p110-d fusion protein is assembled in a third PCR reaction by the overlapping linkers at the 3′ end of the iSH2 fragment and the 5′ end of the p110-d fragment, using the above mentioned gwG130-p03 primer and a primer containing an overlapping Histidine tag and the Gateway (Invitrogen) AttB2 recombination sequences (5′-GGGACCACTTTGTA-CAAGAAAGCTGGGTTT-AAGCTCCGTGATGGTGATGGTGATGTGCTCC-3′) (SEQ ID NO: 21).

This final product is recombined in a Gateway (Invitrogen) OR reaction into the donor vector pDONR201 to generate the ORF319 entry clone. This clone is verified by sequencing and used in a Gateway LR reaction to transfer the insert into the Gateway adapted pBlueBac4.5 (Invitrogen) vector for generation of the baculovirus expression vector LR415.

PI3Kγ BV-950 p110g(D144aa)-C-Term His Tag

This construct is obtained from Roger Williams lab, MRC Laboratory of Molecular Biology, Cambridge, UK (November, 2003). Description of the construct in: Pacold M. E. et al. (2000) Cell 103, 931-943.

### Expression:

### Methods to Generate Recombinant Baculovirus and Protein for PI3K Isoforms:

The pBlue-Bac4.5 (for a, b, and d isoforms) or pVL1393 (for g) plasmids containing the different PI3 kinase genes are co-transfected with BaculoGold WT genomic DNA (BD Biosciences, Franklin Lakes, N.J., USA) using methods recommended by the vendor. Subsequently, the recombinant baculovirus obtained from the transfection is plaque-purified on Sf9 insect cells to yield several isolates expressing recombinant protein. Positive clones are selected by anti-HIS or anti-isoform antibody western. For PI3K alpha and delta isoforms, a secondary plaque-purification is performed on the first clonal virus stocks of PI3K. Amplification of all baculovirus isolates is performed at low multiplicity of infection (moi) to generate high-titer, low passage stock for protein production. The baculoviruses are designated BV1052 (a) and BV1075 (a), BV949 (β), BV1060 (δ) and BV950 (γ). Protein production involves infection (passage 3 or lower) of suspended Tn5 (Trichoplusia ni) or TiniPro (Expression Systems, LLC, Woodland, Calif., USA) cells in protein-free media at moi of 2-10 for 39-48 hours in 2 L glass Erlenmyer flasks (110 rpm) or wave-bioreactors (22-25 rpm). Initially, 10 L working volume wave-bioreactors are seeded at a density of 3e5 cells/ml at half capacity (5 L). The reactor is rocked at 15 rpm during the cell growth phase for 72 hours, supplemented with 5% oxygen mixed with air (0.2 L per minute). Immediately prior to infection, the wave-reactor cultures are analyzed for density, viability and diluted to approximately 1.5e6 cell/ml. 100-500 ml of high titer, low passage virus is added following 2-4 hours of additional culture. Oxygen is increased to 35% for the 39-48 hour infection period and rocking platform rpm increased to 25. During infection, cells are monitored by Vicell viability analyzer (Beckman Coulter, Inc, Fullerton, Calif., USA) bioprocess for viability, diameter and density. Nova Bioanalyzer (NOVA Biomedical Corp., Waltham, Mass., USA) readings of various parameters and metabolites (pH, O₂ saturation, glucose, etc.) are taken every 12-18 hours until harvest. The wave-bioreactor cells are collected within 40 hours post infection. Cells are collected by centrifugation (4 degrees C. at 1500 rpm), and subsequently maintained on ice during pooling of pellets for lysis and purification. Pellet pools are made with small amounts of cold, un-supplemented Grace's media (w/o protease inhibitors).

### PI3K Alpha Purification Protocol for HTS (BV1052)

PI3K alpha is purified in three chromatographic steps: immobilized metal affinity chromatography on a Ni Sepharose resin (GE Healthcare, belonging to General Electric Company, Fairfield, Conn., USA), gel filtration utilizing a Superdex 200 26/60 column (GE Healthcare), and finally a cation exchange step on a SP-XL column (GE Healthcare). All buffers are chilled to 4° C. and lysis is performed chilled on ice. Column fractionation is performed rapidly at room temperature.

Typically frozen insect cells are lysed in a hypertonic lysis buffer and applied to a prepared IMAC column. The resin is washed with 3-5 column volumes of lysis buffer, followed by 3-5 column volumes wash buffer containing 45 mM imidazole, and the target protein is then eluted with a buffer containing 250 mM imidazole. Fractions are analyzed by Coomassie stained SDS-PAGE gels, and fractions containing target protein are pooled and applied to a prepared GFC column. Fractions from the GFC column are analyzed by Coomassie stained SDS-PAGE gels, and fractions containing target protein are pooled. The pool from the GFC column is diluted into a low salt buffer and applied to a prepared SP-XL column. The column is washed with low salt buffer until a stable A280 baseline absorbance is achieved, and eluted using a 20 column volume gradient from 0 mM NaCl to 500 mM NaCl. Again, fractions from the SP-XL column are analyzed by Coomassie stained SDS-PAGE gels, and fractions containing the target protein are pooled. The final pool is dialyzed into a storage buffer containing 50% glycerol and stored at −20° C. The final pool is assayed for activity in a phosphoinosititol kinase assay.

PI3K beta Purification Protocol for HTS (BV949)

PI3K beta is purified in two chromatographic steps: immobilized metal affinity chromatography (IMAC) on a Ni Sepharose resin (GE Healthcare) and gel filtration (GFC) utilizing a Superdex 200 26/60 column (GE Healthcare). All buffers are chilled to 4° C. and lysis is performed chilled on ice. Column fractionation is performed rapidly at room temperature.

Typically frozen insect cells are lysed in a hypertonic lysis buffer and applied to a prepared IMAC column. The resin is washed with 3-5 column volumes of lysis buffer, followed by 3-5 column volumes wash buffer containing 45 mM imidazole, and the target protein is then eluted with a buffer containing 250 mM imidazole. Fractions are analyzed by Coomassie stained SDS-PAGE gels, and fractions containing target protein are pooled and applied to a prepared GFC column. Fractions from the GFC column are analyzed by Coomassie stained SDS-PAGE gels, and fractions containing target protein are pooled. The final pool is dialyzed into a storage buffer containing 50% glycerol and stored at −20° C. The final pool is assayed for activity in the phosphoinostitol kinase assay.

### PI3K Gamma Purification Protocol For HTS (BV950)

PI3K gamma is purified in two chromatographic steps: immobilized metal affinity chromatography (IMAC) on a Ni Sepharose resin (GE Healthcare) and gel filtration (GFC) utilizing a Superdex 200 26/60 column (GE Healthcare). All buffers are chilled to 4° C. and lysis is performed chilled on ice. Column fractionation is performed rapidly at room temperature. Typically frozen insect cells are lysed in a hypertonic lysis buffer and applied to a prepared IMAC column. The resin is washed with 3-5 column volumes of lysis buffer, followed by 3-5 column volumes wash buffer containing 45 mM imidazole, and the target protein is then eluted with a buffer containing 250 mM imidazole. Fractions are analyzed by Coomassie stained SDS-PAGE gels, and fractions containing target protein are pooled and applied to a prepared GFC column. Fractions from the GFC column are analyzed by Coomassie stained SDS-PAGE gels, and fractions containing target protein are pooled. The final pool is dialyzed into a storage buffer containing 50% glycerol and stored at −20° C. The final pool is assayed for activity in the phosphoinostitol kinase assay.

PI3K delta Purification Protocol for HTS (BV1060)

PI3K delta is purified in three chromatographic steps: immobilized metal affinity chromatography on a Ni Sepharose resin (GE Healthcare), gel filtration utilizing a Superdex 200 26/60 column (GE Healthcare), and finally a anion exchange step on a Q-HP column (GE Healthcare). All buffers are chilled to 4° C. and lysis is performed chilled on ice. Column fractionation is performed rapidly at room temperature. Typically frozen insect cells are lysed in a hypertonic lysis buffer and applied to a prepared IMAC column. The resin is washed with 3-5 column volumes of lysis buffer, followed by 3-5 column volumes wash buffer containing 45 mM imidazole, and the target protein is then eluted with a buffer containing 250 mM imidazole. Fractions are analyzed by Coomassie stained SDS-PAGE gels, and fractions containing the target protein are pooled and applied to a prepared GFC column. Fractions from the GFC column are analyzed by Coomassie stained SDS-PAGE gels, and fractions containing the target protein are pooled. The pool from the GFC column is diluted into a low salt buffer and applied to a prepared Q-HP column. The column is washed with low salt buffer until a stable A280 baseline absorbance is achieved, and eluted using a 20 column volume gradient from 0 mM NaCl to 500 mM NaCl. Again, fractions from the Q-HP column are analyzed by Coomassie stained SDS-PAGE gels, and fractions containing the target protein are pooled. The final pool is dialyzed into a storage buffer containing 50% glycerol and stored at −20° C. The final pool is assayed for activity in the phosphoinostitol kinase assay.

IC50 is determined by a four parameter curve fitting routine that comes aloneg with “excel fit”. A 4 Parameter logistic equation is used to calculate IC₅₀ values (IDBS XLfit) of the percentage inhibition of each compound at 8 concentrations (usually 10, 3.0, 1.0, 0.3, 0.1, 0.030, 0.010 and 0.003 μM). Alternatively, IC50 values are calculated using idbsXLfit model 204, which is a 4 parameter logistic model.

Yet alternatively, for an ATP depletion assay, compounds of the formula Ito be tested are dissolved in DMSO and directly distributed into a white 384-well plate at 0.5 μL per well. To start the reaction, 10 μL of 10 nM PI3 kinase and 5 μg/mL 1-alpha-phosphatidylinositol (PI) are added into each well followed by 10 μL of 2 μM ATP. The reaction is performed until approx 50% of the ATP is depleted, and then stopped by the addition of 20 μL of Kinase-Glo solution (Promega Corp., Madison, Wis., USA). The stopped reaction is incubated for 5 minutes and the remaining ATP is then detected via luminescence. IC50 values are then determined.

Some of the compounds show a certain level of selectivity against the different paralogs PI3K alpha, beta, gamma and delta.

The range of activity, expressed as IC50, in the assays described above is preferably between 1 nM and about 10 μM, preferably between 1 nM and 3 μM.

### Description of Biochemical Assay for DNA-PK:

The assay is conducted using the kit V7870 from Promega (SignaTECT® DNA-Dependent Protein Kinase System, comprises DNA-PK, biotinylated peptide substrate and further ingredients, Promega, Madison, Wis., USA), that quantitates DNA-dependent protein kinase activity, both in purified enzyme preparations and in cell nuclear extracts. DNA-PK is a nuclear serine/threonine protein kinase that requires double-stranded DNA (dsDNA) for activity. The binding of dsDNA to the enzyme results in the formation of the active enzyme and also brings the substrate closer to the enzyme, allowing the phosphorylation reaction to proceed.

DNA-PK X5 reaction buffer (250 mM HEPES, 500 mM KCl, 50 mM MgCl₂, 1 mM EGTA, 0.5 mM EDTA, 5 mM DTT, pH to 7.5 with KOH) is diluted ⅕ in deionised water and BSA (stock=10 mg/ml) is added to a final concentration of 0.1 mg/ml.

The activation buffer is made from 100 μg/ml of calf thymus DNA in control buffer (10 mM Tris-HCl (pH 7.4), 1 mM EDTA (pH 8.0)). Per tube, the reaction mix is composed of: 2.5 μl of activation or control buffers, 5 μl of X5 reaction buffer, 2.5 μl of p53-derived biotinylated peptide substrate (stock=4 mM), 0.2 μl of BSA (stock at 10 mg/ml) and 5 μl of [γ-³²P] ATP (5 μl of 0.5 mM cold ATP+0.05 μl of Redivue [γ-³²P] ATP=Amersham AA0068-250 μCi, 3000 Ci/mmol, 10 μCi/μl (now GE Gealthcare Biosciences AB, Uppsala, Sweden).

The DNA-PK enzyme (Promega V5811, concentration=100 U/μL) is diluted 1/10 in X1 reaction buffer and kept on ice until imminent use. 10.8 μl of the diluted enzyme is incubated with 1.2 μl of 100 μM compounds (diluted 1/100 in water from 10 mM stock in neat DMSO) for 10 minutes, at room temperature. During that time, 15.2 μl of the reaction mix is added to screw-capped tubes, behind Perspex glass. 9.8 μl of the enzyme is then transferred to the tubes containing the reaction mix and after 5 minutes incubation, at 30° C., the reaction is stopped by adding 12.5 μl of termination buffer (7.5 M guanidine hydrochloride).

After mixing well, a 10 μl aliquot of each tube is spotted onto a SAM2® biotin capture membrane (Promega, Madison, Wis., USA), which is left to dry for a few minutes. The membrane is then washed extensively to remove the excess free [γ-³²P] ATP and nonbiotinylated proteins: once for 30 seconds in 200 ml of 2M NaCl, 3 times for 2 minutes each in 200 ml of 2M NaCl, 4 times for 2 minutes each in 2M NaCl in 1% H₃PO₄ and twice for 30 seconds each in 100 ml of deionised water. The membrane is subsequently left to air-dry at room temperature for 30-60 minutes.

Each membrane square is separated using forceps and scissors and placed into a scintillation vial, then 8 ml of scintillation liquid (Flo-Scint 6013547 from Perkin-Elmer) is added. The amount of ³²P incorporated into the DNA-PK biotinylated peptide substrate is then determined by liquid scintillation counting. In this test system, compounds of the formula I can be shown to have IC₅₀ values in the range from 10 nM to 50 μM, e.g. from 10 nM to 10 μM.

The efficacy of the compounds of the invention in blocking the activation of the PI3K/PKB pathway can be demonstrated in cellular settings as follows:

### Protocol for the Detection of Phospho-PKB in U87MG Cells by Elisa:

U87MG cells (human glioblastoma, ATCC No. HTB-14) are trypsinized, counted in a CASY cell counter (Schärffe systems, Göttingen, Germany), diluted in fresh complete DMEM high glucose medium to load, per well, 150 μL cell suspension containing 4×10⁴ cells, and test plates incubated for 18 hours. In parallel, 504 of coating antibody, at the desired concentration in PBS/O is loaded in each well of the ELISA plates, and plates are kept for 2 h at room temperature. This ELISA assays is performed in black flat-bottom 96-well plates (Microtesr, Falcon Becton-Dickinson, Ref: 353941) sealed with Plate Sealers (Costar-Corning, Ref: 3095). Medium in plates is discarded and replaced by complete DMEM high glucose medium containing either 0.1% DMSO or 0.1% inhibitor at titers (7) between 10 mM and 0.156 mM in DMSO. After 30 minutes of contact, the medium is quickly removed by aspiration, plates are then placed on ice and immediately cells lyzed with 70 μL of Lysis buffer. In parallel, the 96 wells plates prepared with the coating antibody ( 1/250 diluted (in PBS/O) Anti-Akt1 C-20, goat, Santa-Cruz-1618, Santa Cruz Biotechnology, Inc., Santa Cruz, Calif., USA) are washed 3 times 1 min with PBS/0 containing 0.05% Tween 20 and 0.1% Top-Block® (derivative of gelatine that blocks unspecific binding sites on surfaces; Sigma-Aldrich, Fluka, Buchs, Switzerland, Ref.: 37766), and remaining protein binding sites blocked to prevent non-specific interactions with 200 μL of PBS containing 3% Top Block®, for 2 h at room temperature. Well content is replaced with 50 μL of samples from treated cells, and plates are incubated for 3 h at 4° C. The ELISA assays are always done in parallel with the following controls, in 6 replicates: U87MG (untreated control) or Lysis buffer alone (LB). After 3×15 minutes washes, all wells received 50 μL of the secondary antibody ( 1/250 diluted (in 3% top block) Anti-S473P-PKB, rabbit, Cell Signaling-9271, Cell Signaling Technologies, Inc., Danvers, Mass., USA)), and are incubated for 16 h at 4° C. After three washes, plates are incubated with the third and conjugated antibody ( 1/1000 diluted (in 3% top block) anti rabbit (HRP) Jackson Immuno Research 111-035-144) for 2 hours at room temperature. Finally, the immune-complexes are washed 2 times 15 seconds with PBS/0/tween20/top block, 1 time with 200 μl of water and finally 200 μl of water are left in each test well before a for 45 min incubation in darkness. The plates are then assayed with (SuperSignal® ELISA pico Chemiluminescent substrate, Pierce, Ref: 27070, Pierce Biotechnology, Inc., Rockford, Ill., USA). 100 μL of substrate are added, and plates shacked for 1 min. The luminescence is read immediately on a Top-Count NXT (Packard Bioscience) luminometer. Using this test system, IC₅₀ values in the range from 10 μM to 5 nM, more preferably from 5 μM to 10 nM can be found for compounds of the formula I as test compounds.

There are also experiments that can demonstrate the antitumor activity of compounds of the formula (I) in vivo.

For example, female Harlan (Indianapolis, Ind., USA) athymic nu/nu mice with s.c. trans-planted human glioblastoms U87MG tumors can be used to determine the anti-tumor activity of PI3 kinase inhibitors. On day 0, with the animals under peroral Forene® (1-chloro-2,2,2-trifluoroethyldifluormethylether, Abbot, Wiesbaden, Germany) narcosis, a tumor fragment of approximately 25 mg is placed under the skin on the animals' left flank and the small incised wound is closed by means of suture clips. When tumors reach a volume of 100 mm³, the mice are divided at random into groups of 6-8 animals and treatment commences. The treatment is carried out for a 2-3 weeks period with peroral, intravenous or intra-peritoneal administration once daily (or less frequently) of a compound of formula (I) in a suitable vehicle at defined doses. The tumors are measured twice a week with a slide gauge and the volume of the tumors is calculated.

As an alternative to cell line U87MG, other cell lines may also be used in the same manner, for example,
the MDA-MB 468 breast adenocarcinoma cell line (ATCC No. HTB 132; see also In Vitro 14, 911-15 [1978]);
the MDA-MB 231 breast carcinoma cell line (ATCC No. HTB-26; see also In Vitro 12, 331 [1976]);
the MDA-MB 453 breast carcinoma cell line (ATCC No. HTB-131);
the Colo 205 colon carcinoma cell line (ATCC No. CCL 222; see also Cancer Res. 38, 1345-55 [1978]);
the DU145 prostate carcinoma cell line DU 145 (ATCC No. HTB 81; see also Cancer Res. 37, 4049-58 [1978]),
the PC-3 prostate carcinoma cell line PC-3 (especially preferred; ATCC No. CRL 1435; see also Cancer Res. 40, 524-34 [1980]) and the PC-3M prostate carcinoma cell line;
the A549 human lung adenocarcinoma (ATCC No. CCL 185; see also Int. J. Cancer 17, 62-70 [1976]),
the NCI-H596 cell line (ATCC No. HTB 178; see also Science 246, 491-4 [1989]);
the pancreatic cancer cell line SUIT-2 (see Tomioka et al., Cancer Res. 61, 7518-24 [2001]).

Compounds of the invention exhibit T cell inhibiting activity. More particular the compounds of the invention prevent T cell activation and/or proliferation in e.g. aqueous solution, e.g. as demonstrated in accordance with the following test method. The two-way MLR is performed according to standard procedures (J. Immunol. Methods, 1973, 2, 279 and Meo T. et al., Immunological Methods, New York, Academic Press, 1979, 227-39). Briefly, spleen cells from CBA and BALB/c mice (1.6×105 cells from each strain per well in flat bottom tissue culture microtiter plates, 3.2×105 in total) are incubated in RPMI medium containing 10% FCS, 100 U/ml penicillin, 100 μg/ml streptomycin (Gibco BRL, Basel, Switzerland), 50 μM 2-mercaptoethanol (Fluka, Buchs, Switzerland) and serially diluted compounds. Seven three-fold dilution steps in duplicates per test compound are performed. After four days of incubation, 1 μCi 3H-thymidine is added. Cells are harvested after an additional five-hour incubation period, and incorporated 3H-thymidine is determined according to standard procedures. Background values (low control) of the MLR are the proliferation of BALB/c cells alone. Low controls are subtracted from all values. High controls without any sample are taken as 100% proliferation. Percent inhibition by the samples is calculated, and the concentrations required for 50% inhibition (IC50 values) are determined. In this assay, the compounds of the invention preferably have IC50 values in the range of 10 nM to 5 μM, preferably from 10 nM to 500 nM.

A compound of the formula (I) may also be used to advantage in combination with other anti-proliferative compounds. Such antiproliferative compounds include, but are not limited to aromatase inhibitors; antiestrogens; topoisomerase I inhibitors; topoisomerase II inhibitors; microtubule active compounds; alkylating compounds; histone deacetylase inhibitors; compounds which induce cell differentiation processes; cyclooxygenase inhibitors; MMP inhibitors; mTOR inhibitors; antineoplastic antimetabolites; platin compounds; compounds targeting/decreasing a protein or lipid kinase activity and further anti-angiogenic compounds; compounds which target, decrease or inhibit the activity of a protein or lipid phosphatase; gonadorelin agonists; anti-androgens; methionine aminopeptidase inhibitors; bisphosphonates; biological response modifiers; antiproliferative antibodies; heparanase inhibitors; inhibitors of Ras oncogenic isoforms; telomerase inhibitors; proteasome inhibitors; compounds used in the treatment of hematologic malignancies; compounds which target, decrease or inhibit the activity of Flt-3; Hsp90 inhibitors such as 17-AAG (17-allylaminogeldanamycin, NSC330507), 17-DMAG (17-dimethylaminoethylamino-17-demethoxy-geldanamycin, NSC707545), IPI-504, CNF1010, CNF2024, CNF1010 from Conforma Therapeutics; temozolomide (TEMODAL®); kinesin spindle protein inhibitors, such as SB715992 or SB743921 from GlaxoSmithKline, or pentamidine/chlorpromazine from CombinatoRx; MEK inhibitors such as ARRY142886 from Array PioPharma, AZD6244 from AstraZeneca, PD181461 from Pfizer, leucovorin, EDG binders, antileukemia compounds, ribonucleotide reductase inhibitors, S-adenosylmethionine decarboxylase inhibitors, antiproliferative antibodies or other chemotherapeutic compounds. Further, alternatively or in addition they may be used in combination with other tumor treatment approaches, including surgery, ionizing radiation, photodynamic therapy, implants, e.g. with corticosteroids, hormones, or they may be used as radiosensitizers. Also, in anti-inflammatory and/or antiproliferative treatment, combination with anti-inflammatory drugs is included. Combination is also possible with antihistamine drug substances, bronchodilatatory drugs, NSAID or antagonists of chemokine receptors.

The term “aromatase inhibitor” as used herein relates to a compound which inhibits the estrogen production, i.e. the conversion of the substrates androstenedione and testosterone to estrone and estradiol, respectively. The term includes, but is not limited to steroids, especially atamestane, exemestane and formestane and, in particular, non-steroids, especially aminoglutethimide, roglethimide, pyridoglutethimide, trilostane, testolactone, ketokonazole, vorozole, fadrozole, anastrozole and letrozole. Exemestane can be administered, e.g., in the form as it is marketed, e.g. under the trademark AROMASIN. Formestane can be administered, e.g., in the form as it is marketed, e.g. under the trademark LENTARON. Fadrozole can be administered, e.g., in the form as it is marketed, e.g. under the trademark AFEMA. Anastrozole can be administered, e.g., in the form as it is marketed, e.g. under the trademark ARIMIDEX. Letrozole can be administered, e.g., in the form as it is marketed, e.g. under the trademark FEMARA or FEMAR. Aminoglutethimide can be administered, e.g., in the form as it is marketed, e.g. under the trademark ORIMETEN. A combination of the invention comprising a chemotherapeutic agent which is an aromatase inhibitor is particularly useful for the treatment of hormone receptor positive tumors, e.g. breast tumors.

The term “antiestrogen” as used herein relates to a compound which antagonizes the effect of estrogens at the estrogen receptor level. The term includes, but is not limited to tamoxifen, fulvestrant, raloxifene and raloxifene hydrochloride. Tamoxifen can be administered, e.g., in the form as it is marketed, e.g. under the trademark NOLVADEX. Raloxifene hydrochloride can be administered, e.g., in the form as it is marketed, e.g. under the trademark EVISTA. Fulvestrant can be formulated as disclosed in U.S. Pat. No. 4,659,516 or it can be administered, e.g., in the form as it is marketed, e.g. under the trademark FASLODEX. A combination of the invention comprising a chemotherapeutic agent which is an antiestrogen is particularly useful for the treatment of estrogen receptor positive tumors, e.g. breast tumors.

The term “anti-androgen” as used herein relates to any substance which is capable of inhibiting the biological effects of androgenic hormones and includes, but is not limited to, bicalutamide (CASODEX), which can be formulated, e.g. as disclosed in U.S. Pat. No. 4,636,505.

The term “gonadorelin agonist” as used herein includes, but is not limited to abarelix, goserelin and goserelin acetate. Goserelin is disclosed in U.S. Pat. No. 4,100,274 and can be administered, e.g., in the form as it is marketed, e.g. under the trademark ZOLADEX. Abarelix can be formulated, e.g. as disclosed in U.S. Pat. No. 5,843,901.

The term “topoisomerase I inhibitor” as used herein includes, but is not limited to topotecan, gimatecan, irinotecan, camptothecian and its analogues, 9-nitrocamptothecin and the macromolecular camptothecin conjugate PNU-166148 (compound A1 in WO99/17804). Irinotecan can be administered, e.g. in the form as it is marketed, e.g. under the trademark CAMPTOSAR. Topotecan can be administered, e.g., in the form as it is marketed, e.g. under the trademark HYCAMTIN.

The term “topoisomerase II inhibitor” as used herein includes, but is not limited to the anthracyclines such as doxorubicin (including liposomal formulation, e.g. CAELYX), daunorubicin, epirubicin, idarubicin and nemorubicin, the anthraquinones mitoxantrone and losoxantrone, and the podophillotoxines etoposide and teniposide. Etoposide can be administered, e.g. in the form as it is marketed, e.g. under the trademark ETOPOPHOS. Teniposide can be administered, e.g. in the form as it is marketed, e.g. under the trademark VM 26-BRISTOL. Doxorubicin can be administered, e.g. in the form as it is marketed, e.g. under the trademark ADRIBLASTIN or ADRIAMYCIN. Epirubicin can be administered, e.g. in the form as it is marketed, e.g. under the trademark FARMORUBICIN. Idarubicin can be administered, e.g. in the form as it is marketed, e.g. under the trademark ZAVEDOS. Mitoxantrone can be administered, e.g. in the form as it is marketed, e.g. under the trademark NOVANTRON.

The term “microtubule active compound” relates to microtubule stabilizing, microtubule destabilizing compounds and microtublin polymerization inhibitors including, but not limited to taxanes, e.g. paclitaxel and docetaxel, vinca alkaloids, e.g., vinblastine, especially vinblastine sulfate, vincristine especially vincristine sulfate, and vinorelbine, discodermolides, colchicine and epothilones and derivatives thereof, e.g. epothilone B or D or derivatives thereof. Paclitaxel may be administered e.g. in the form as it is marketed, e.g. TAXOL. Docetaxel can be administered, e.g., in the form as it is marketed, e.g. under the trademark TAXOTERE. Vinblastine sulfate can be administered, e.g., in the form as it is marketed, e.g. under the trademark VINBLASTIN R.P. Vincristine sulfate can be administered, e.g., in the form as it is marketed, e.g. under the trademark FARMISTIN. Discodermolide can be obtained, e.g., as disclosed in U.S. Pat. No. 5,010,099. Also included are Epothilone derivatives which are disclosed in WO 98/10121, U.S. Pat. No. 6,194,181, WO 98/25929, WO 98/08849, WO 99/43653, WO 98/22461 and WO 00/31247. Especially preferred are Epothilone A and/or B.

The term “alkylating compound” as used herein includes, but is not limited to, cyclophosphamide, ifosfamide, melphalan or nitrosourea (BCNU or Gliadel). Cyclophosphamide can be administered, e.g., in the form as it is marketed, e.g. under the trademark CYCLOSTIN. Ifosfamide can be administered, e.g., in the form as it is marketed, e.g. under the trademark HOLOXAN.

The term “histone deacetylase inhibitors” or “HDAC inhibitors” relates to compounds which inhibit the histone deacetylase and which possess antiproliferative activity. This includes compounds disclosed in WO 02/22577, especially N-hydroxy-3-[4-[[(2-hydroxyethyl)[2-(1H-indol-3-ypethyl]-amino]methyl]phenyl]-2E-2-propenamide, N-hydroxy-3-[4-[[[2-(2-methyl-1H-indol-3-yl)-ethyl]-amino]methyl]phenyl]-2E-2-propenamide and pharmaceutically acceptable salts thereof. It further especially includes Suberoylanilide hydroxamic acid (SAHA).

The term “antineoplastic antimetabolite” includes, but is not limited to, 5-Fluorouracil or 5-FU, capecitabine, gemcitabine, DNA demethylating compounds, such as 5-azacytidine and decitabine, methotrexate and edatrexate, and folic acid antagonists such as pemetrexed. Capecitabine can be administered, e.g., in the form as it is marketed, e.g. under the trademark XELODA. Gemcitabine can be administered, e.g., in the form as it is marketed, e.g. under the trademark GEMZAR.

The term “platin compound” as used herein includes, but is not limited to, carboplatin, cisplatin, cisplatinum and oxaliplatin. Carboplatin can be administered, e.g., in the form as it is marketed, e.g. under the trademark CARBOPLAT. Oxaliplatin can be administered, e.g., in the form as it is marketed, e.g. under the trademark ELOXATIN.

The term “compounds targeting/decreasing a protein or lipid kinase activity”; or a “protein or lipid phosphatase activity”; or “further anti-angiogenic compounds” as used herein includes, but is not limited to, protein tyrosine kinase and/or serine and/or threonine kinase inhibitors or lipid kinase inhibitors, e.g.,
a) compounds targeting, decreasing or inhibiting the activity of the platelet-derived growth factor-receptors (PDGFR), such as compounds which target, decrease or inhibit the activity of PDGFR, especially compounds which inhibit the PDGF receptor, e.g. a N-phenyl-2-pyrimidine-amine derivative, e.g. imatinib, SU101, SU6668 and GFB-111;
b) compounds targeting, decreasing or inhibiting the activity of the fibroblast growth factor-receptors (FGFR);
c) compounds targeting, decreasing or inhibiting the activity of the insulin-like growth factor receptor I (IGF-IR), such as compounds which target, decrease or inhibit the activity of IGF-IR, especially compounds which inhibit the kinase activity of IGF-I receptor, such as those compounds disclosed in WO 02/092599, or antibodies that target the extracellular domain of IGF-I receptor or its growth factors;
d) compounds targeting, decreasing or inhibiting the activity of the Trk receptor tyrosine kinase family, or ephrin B4 inhibitors;
e) compounds targeting, decreasing or inhibiting the activity of the Axl receptor tyrosine kinase family;
f) compounds targeting, decreasing or inhibiting the activity of the Ret receptor tyrosine kinase;
g) compounds targeting, decreasing or inhibiting the activity of the Kit/SCFR receptor tyrosine kinase, e.g. imatinib;
h) compounds targeting, decreasing or inhibiting the activity of the C-kit receptor tyrosine kinases—(part of the PDGFR family), such as compounds which target, decrease or inhibit the activity of the c-Kit receptor tyrosine kinase family, especially compounds which inhibit the c-Kit receptor, e.g. imatinib;
i) compounds targeting, decreasing or inhibiting the activity of members of the c-Abl family, their gene-fusion products (e.g. BCR-Abl kinase) and mutants, such as compounds which target decrease or inhibit the activity of c-Abl family members and their gene fusion products, e.g. a N-phenyl-2-pyrimidine-amine derivative, e.g. imatinib or nilotinib (AMN107); PD180970; AG957; NSC 680410; PD173955 from ParkeDavis; or dasatinib (BMS-354825)
j) compounds targeting, decreasing or inhibiting the activity of members of the protein kinase C (PKC) and Raf family of serine/threonine kinases, members of the MEK, SRC, JAK, FAK, PDK1, PKB/Akt, and Ras/MAPK family members, and/or members of the cyclin-dependent kinase family (CDK) and are especially those staurosporine derivatives disclosed in U.S. Pat. No. 5,093,330, e.g. midostaurin; examples of further compounds include e.g. UCN-01, safingol, BAY 43-9006, Bryostatin 1, Perifosine; Ilmofosine; RO 318220 and RO 320432; GO 6976; Isis 3521; LY333531/LY379196; isochinoline compounds such as those disclosed in WO 00/09495; FTIs; PD184352 or QAN697 (a PI3K inhibitor) or AT7519 (CDK inhibitor);
k) compounds targeting, decreasing or inhibiting the activity of protein-tyrosine kinase inhibitors, such as compounds which target, decrease or inhibit the activity of protein-tyrosine kinase inhibitors include imatinib mesylate (GLEEVEC) or tyrphostin. A tyrphostin is preferably a low molecular weight (Mr<1500) compound, or a pharmaceutically acceptable salt thereof, especially a compound selected from the benzylidenemalonitrile class or the S-arylbenzenemalonirile or bisubstrate quinoline class of compounds, more especially any compound selected from the group consisting of Tyrphostin A23/RG-50810; AG 99; Tyrphostin AG 213; Tyrphostin AG 1748; Tyrphostin AG 490; Tyrphostin B44; Tyrphostin B44 (+) enantiomer; Tyrphostin AG 555; AG 494; Tyrphostin AG 556, AG957 and adaphostin (4-{[(2,5-dihydroxyphenyl)methyl]amino}-benzoic acid adamantyl ester; NSC 680410, adaphostin);
l) compounds targeting, decreasing or inhibiting the activity of the epidermal growth factor family of receptor tyrosine kinases (EGFR, ErbB2, ErbB3, ErbB4 as homo- or heterodimers) and their mutants, such as compounds which target, decrease or inhibit the activity of the epidermal growth factor receptor family are especially compounds, proteins or antibodies which inhibit members of the EGF receptor tyrosine kinase family, e.g. EGF receptor, ErbB2, ErbB3 and ErbB4 or bind to EGF or EGF related ligands, and are in particular those compounds, proteins or monoclonal antibodies generically and specifically disclosed in WO 97/02266, e.g. the compound of ex. 39, or in EP 0 564 409, WO 99/03854, EP 0520722, EP 0 566 226, EP 0 787 722, EP 0 837 063, U.S. Pat. No. 5,747,498, WO 98/10767, WO 97/30034, WO 97/49688, WO 97/38983 and, especially, WO 96/30347 (e.g. compound known as CP 358774), WO 96/33980 (e.g. compound ZD 1839) and WO 95/03283 (e.g. compound ZM105180); e.g. trastuzumab (Herceptin™), cetuximab (Erbitux™), Iressa, Tarceva, OSI-774, CI-1033, EKB-569, GW-2016, E1.1, E2.4, E2.5, E6.2, E6.4, E2.11, E6.3 or E7.6.3, and 7H-pyrrolo-[2,3-d]pyrimidine derivatives which are disclosed in WO 03/013541; and
m) compounds targeting, decreasing or inhibiting the activity of the c-Met receptor, such as compounds which target, decrease or inhibit the activity of c-Met, especially compounds which inhibit the kinase activity of c-Met receptor, or antibodies that target the extracellular domain of c-Met or bind to HGF.

Further anti-angiogenic compounds include compounds having another mechanism for their activity, e.g. unrelated to protein or lipid kinase inhibition e.g. thalidomide (THALOMID) and TNP-470.

Compounds which target, decrease or inhibit the activity of a protein or lipid phosphatase are e.g. inhibitors of phosphatase 1, phosphatase 2A, or CDC25, e.g. okadaic acid or a derivative thereof.

Compounds which induce cell differentiation processes are e.g. retinoic acid, α- γ- or δ-tocopherol or α- γ- or δ-tocotrienol.

The term cyclooxygenase inhibitor as used herein includes, but is not limited to, e.g. Cox-2 inhibitors, 5-alkyl substituted 2-arylaminophenylacetic acid and derivatives, such as celecoxib (CELEBREX), rofecoxib (VIOXX), etoricoxib, valdecoxib or a 5-alkyl-2-arylaminophenylacetic acid, e.g. 5-methyl-2-(2′-chloro-6′-fluoroanilino)phenyl acetic acid, lumiracoxib.

The term “bisphosphonates” as used herein includes, but is not limited to, etridonic, clodronic, tiludronic, pamidronic, alendronic, ibandronic, risedronic and zoledronic acid. “Etridonic acid” can be administered, e.g., in the form as it is marketed, e.g. under the trademark DIDRONEL. “Clodronic acid” can be administered, e.g., in the form as it is marketed, e.g. under the trademark BONEFOS. “Tiludronic acid” can be administered, e.g., in the form as it is marketed, e.g. under the trademark SKELID. “Pamidronic acid” can be administered, e.g. in the form as it is marketed, e.g. under the trademark AREDIA™. “Alendronic acid” can be administered, e.g., in the form as it is marketed, e.g. under the trademark FOSAMAX. “Ibandronic acid” can be administered, e.g., in the form as it is marketed, e.g. under the trademark BONDRANAT. “Risedronic acid” can be administered, e.g., in the form as it is marketed, e.g. under the trademark ACTONEL. “Zoledronic acid” can be administered, e.g. in the form as it is marketed, e.g. under the trademark ZOMETA.

The term “mTOR inhibitors” relates to compounds which inhibit the mammalian target of rapamycin (mTOR) and which possess antiproliferative activity such as sirolimus (Rapamune®), everolimus (Certican™), CCI-779 and ABT578.

The term “heparanase inhibitor” as used herein refers to compounds which target, decrease or inhibit heparin sulfate degradation. The term includes, but is not limited to, PI-88.

The term “biological response modifier” as used herein refers to a lymphokine or interferons, e.g. interferon γ.

The term “inhibitor of Ras oncogenic isoforms”, e.g. H-Ras, K-Ras, or N-Ras, as used herein refers to compounds which target, decrease or inhibit the oncogenic activity of Ras e.g. a “farnesyl transferase inhibitor” e.g. L-744832, DK8G557 or R115777 (Zarnestra).

The term “telomerase inhibitor” as used herein refers to compounds which target, decrease or inhibit the activity of telomerase. Compounds which target, decrease or inhibit the activity of telomerase are especially compounds which inhibit the telomerase receptor, e.g. telomestatin.

The term “methionine aminopeptidase inhibitor” as used herein refers to compounds which target, decrease or inhibit the activity of methionine aminopeptidase. Compounds which target, decrease or inhibit the activity of methionine aminopeptidase are e.g. bengamide or a derivative thereof.

The term “proteasome inhibitor” as used herein refers to compounds which target, decrease or inhibit the activity of the proteasome. Compounds which target, decrease or inhibit the activity of the proteasome include e.g. Bortezomid (Velcade™) and MLN 341.

The term “matrix metalloproteinase inhibitor” or (“MMP” inhibitor) as used herein includes, but is not limited to, collagen peptidomimetic and nonpeptidomimetic inhibitors, tetracycline derivatives, e.g. hydroxamate peptidomimetic inhibitor batimastat and its orally bioavailable analogue marimastat (BB-2516), prinomastat (AG3340), metastat (NSC 683551) BMS-279251, BAY 12-9566, TAA211, MMI270B or AAJ996.

The term “compounds used in the treatment of hematologic malignancies” as used herein includes, but is not limited to, FMS-like tyrosine kinase inhibitors e.g. compounds targeting, decreasing or inhibiting the activity of FMS-like tyrosine kinase receptors (Flt-3R); interferon, 1-b-D-arabinofuransylcytosine (ara-c) and bisulfan; and ALK inhibitors e.g. compounds which target, decrease or inhibit anaplastic lymphoma kinase.

Compounds which target, decrease or inhibit the activity of FMS-like tyrosine kinase receptors (Flt-3R) are especially compounds, proteins or antibodies which inhibit members of the Flt-3R receptor kinase family, e.g. PKC412, midostaurin, a staurosporine derivative, SU11248 and MLN518.

The term “HSP90 inhibitors” as used herein includes, but is not limited to, compounds targeting, decreasing or inhibiting the intrinsic ATPase activity of HSP90; degrading, targeting, decreasing or inhibiting the HSP90 client proteins via the ubiquitin proteosome pathway. Compounds targeting, decreasing or inhibiting the intrinsic ATPase activity of HSP90 are especially compounds, proteins or antibodies which inhibit the ATPase activity of HSP90 e.g., 17-allylamino, 17-demethoxygeldanamycin (17AAG), a geldanamycin derivative; other geldanamycin related compounds; radicicol and HDAC inhibitors.

The term “antiproliferative antibodies” as used herein includes, but is not limited to, trastuzumab (Herceptin™), trastuzumab-DM1, erbitux, bevacizumab (Avastin™), rituximab (Rituxan®), PRO64553 (anti-CD40) and 2C4 Antibody. By antibodies is meant e.g. intact monoclonal antibodies, polyclonal antibodies, multispecific antibodies formed from at least 2 intact antibodies, and antibodies fragments so long as they exhibit the desired biological activity.

For the treatment of acute myeloid leukemia (AML), compounds of formula (I) can be used in combination with standard leukemia therapies, especially in combination with therapies used for the treatment of AML. In particular, compounds of formula (I) can be administered in combination with, e.g., farnesyl transferase inhibitors and/or other drugs useful for the treatment of AML, such as Daunorubicin, Adriamycin, Ara-C, VP-16, Teniposide, Mitoxantrone, Idarubicin, Carboplatinum and PKC412.

The term “antileukemic compounds” includes, for example, Ara-C, a pyrimidine analog, which is the 2″-alpha-hydroxy ribose (arabinoside) derivative of deoxycytidine. Also included is the purine analog of hypoxanthine, 6-mercaptopurine (6-MP) and fludarabine phosphate. Compounds which target, decrease or inhibit activity of histone deacetylase (HDAC) inhibitors such as sodium butyrate and suberoylanilide hydroxamic acid (SAHA) inhibit the activity of the enzymes known as histone deacetylases. Specific HDAC inhibitors include MS275, SAHA, FK228 (formerly FR901228), Trichostatin A and compounds disclosed in U.S. Pat. No. 6,552,065, in particular, N-hydroxy-3-[4-[[[2-(2-methyl-1H-indol-3-yl)-ethyl]-amino]methyl]phenyl]-2E-2-propenamide, or a pharmaceutically acceptable salt thereof and N-hydroxy-3-[4-[(2-hydroxyethyl){2-(1H-indol-3-yl)ethyl]-amino]methyl]phenyl]-2E-2-propenamide, or a pharmaceutically acceptable salt thereof, especially the lactate salt.

Somatostatin receptor antagonists as used herein refers to compounds which target, treat or inhibit the somatostatin receptor such as octreotide, and SOM230 (pasireotide).

Tumor cell damaging approaches refer to approaches such as ionizing radiation. The term “ionizing radiation” referred to above and hereinafter means ionizing radiation that occurs as either electromagnetic rays (such as X-rays and gamma rays) or particles (such as alpha and beta particles). Ionizing radiation is provided in, but not limited to, radiation therapy and is known in the art. See Hellman, Principles of Radiation Therapy, Cancer, in Principles and Practice of Oncology, Devita et al., Eds., 4^{th} Edition, Vol. 1, pp. 248-275 (1993). The term “EDG binders” as used herein refers a class of immunosuppressants that modulates lymphocyte recirculation, such as FTY720.

The term “ribonucleotide reductase inhibitors” refers to pyrimidine or purine nucleoside analogs including, but not limited to, fludarabine and/or cytosine arabinoside (ara-C), 6-thioguanine, 5-fluorouracil, cladribine, 6-mercaptopurine (especially in combination with ara-C against ALL) and/or pentostatin. Ribonucleotide reductase inhibitors are especially hydroxyurea or 2-hydroxy-1H-isoindole-1,3-dione derivatives, such as PL-1, PL-2, PL-3, PL-4, PL-5, PL-6, PL-7 or PL-8 mentioned in Nandy et al., Acta Oncologica, Vol. 33, No. 8, pp. 953-961 (1994).

The term “S-adenosylmethionine decarboxylase inhibitors” as used herein includes, but is not limited to the compounds disclosed in U.S. Pat. No. 5,461,076.

Also included are in particular those compounds, proteins or monoclonal antibodies of VEGF disclosed in WO 98/35958, e.g. 1-(4-chloroanilino)-4-(4-pyridylmethyl)phthalazine or a pharmaceutically acceptable salt thereof, e.g. the succinate, or in WO 00/09495, WO 00/27820, WO 00/59509, WO 98/11223, WO 00/27819 and EP 0 769 947; those as described by Prewett et al, Cancer Res, Vol. 59, pp. 5209-5218 (1999); Yuan et al., Proc Natl Acad Sci USA, Vol. 93, pp. 14765-14770 (1996); Zhu et al., Cancer Res, Vol. 58, pp. 3209-3214 (1998); and Mordenti et al., Toxicol Pathol, Vol. 27, No. 1, pp. 14-21 (1999); in WO 00/37502 and WO 94/10202; ANGIOSTATIN, described by O'Reilly et al., Cell, Vol. 79, pp. 315-328 (1994); ENDOSTATIN, described by O'Reilly et al., Cell, Vol. 88, pp. 277-285 (1997); anthranilic acid amides; ZD4190; ZD6474; SU5416; SU6668; bevacizumab; or anti-VEGF antibodies or anti-VEGF receptor antibodies, e.g. rhuMAb and RHUFab, VEGF aptamer e.g. Macugon; FLT-4 inhibitors, FLT-3 inhibitors, VEGFR-2 IgG1 antibody, Angiozyme (RPI 4610) and Bevacizumab (Avastin™).

Photodynamic therapy as used herein refers to therapy which uses certain chemicals known as photosensitizing compounds to treat or prevent cancers. Examples of photodynamic therapy includes treatment with compounds, such as e.g. VISUDYNE and porfimer sodium.

Angiostatic steroids as used herein refers to compounds which block or inhibit angiogenesis, such as, e.g., anecortave, triamcinolone. hydrocortisone, 11-α-epihydrocotisol, cortexolone, 17α-hydroxyprogesterone, corticosterone, desoxycorticosterone, testosterone, estrone and dexamethasone.

Implants containing corticosteroids refers to compounds, such as e.g. fluocinolone, dexamethasone.

“Other chemotherapeutic compounds” include, but are not limited to, plant alkaloids, hormonal compounds and antagonists; biological response modifiers, preferably lymphokines or interferons; antisense oligonucleotides or oligonucleotide derivatives; shRNA or siRNA; or miscellaneous compounds or compounds with other or unknown mechanism of action.

The compounds of the invention are also useful as co-therapeutic compounds for use in combination with other drug substances such as anti-inflammatory, bronchodilatory or anti-histamine drug substances, particularly in the treatment of obstructive or inflammatory air-ways diseases such as those mentioned hereinbefore, for example as potentiators of therapeutic activity of such drugs or as a means of reducing required dosaging or potential side effects of such drugs. A compound of the invention may be mixed with the other drug substance in a fixed pharmaceutical composition or it may be administered separately, before, simultaneously with or after the other drug substance. Accordingly the invention includes a combination of a compound of the invention as hereinbefore described with an anti-inflammatory, bronchodilatory, antihistamine or anti-tussive drug substance, said compound of the invention and said drug substance being in the same or different pharmaceutical composition.

Suitable anti-inflammatory drugs include steroids, in particular glucocorticosteroids such as budesonide, beclamethasone dipropionate, fluticasone propionate, ciclesonide or mometasone furoate, or steroids described in WO 02/88167, WO 02/12266, WO 02/100879, WO 02/00679 (especially those of Examples 3, 11, 14, 17, 19, 26, 34, 37, 39, 51, 60, 67, 72, 73, 90, 99 and 101), WO 03/035668, WO 03/048181, WO 03/062259, WO 03/064445, WO 03/072592, non-steroidal glucocorticoid receptor agonists such as those described in WO 00/00531, WO 02/10143, WO 03/082280, WO 03/082787, WO 03/104195, WO 04/005229;

LTB4 antagonists such LY293111, CGS025019C, CP-195543, SC-53228, BIIL 284, ONO 4057, SB 209247 and those described in U.S. Pat. No. 5,451,700; LTD4 antagonists such as montelukast and zafirlukast; PDE4 inhibitors such cilomilast (Ariflo® GlaxoSmithKline), Roflumilast (Byk Gulden), V-11294A (Napp), BAY19-8004 (Bayer), SCH-351591 (Schering-Plough), Arofylline (Almirall Prodesfarma), PD189659/PD168787 (Parke-Davis), AWD-12-281 (Asta Medica), CDC-801 (Celgene), SeICID™ CC-10004 (Celgene), VM554/UM565 (Vernalis), T-440 (Tanabe), KW-4490 (Kyowa Hakko Kogyo), and those disclosed in WO 92/19594, WO 93/19749, WO 93/19750, WO 93/19751, WO 98/18796, WO 99/16766, WO 01/13953, WO 03/104204, WO 03/104205, WO 03/39544, WO 04/000814, WO 04/000839, WO 04/005258, WO 04/018450, WO 04/018451, WO 04/018457, WO 04/018465, WO 04/018431, WO 04/018449, WO 04/018450, WO 04/018451, WO 04/018457, WO 04/018465, WO 04/019944, WO 04/019945, WO 04/045607 and WO 04/037805; A2a agonists such as those disclosed in EP 409595A2, EP 1052264, EP 1241176, WO 94/17090, WO 96/02543, WO 96/02553, WO 98/28319, WO 99/24449, WO 99/24450, WO 99/24451, WO 99/38877, WO 99/41267, WO 99/67263, WO 99/67264, WO 99/67265, WO 99/67266, WO 00/23457, WO 00/77018, WO 00/78774, WO 01/23399, WO 01/27130, WO 01/27131, WO 01/60835, WO 01/94368, WO 02/00676, WO 02/22630, WO 02/96462, WO 03/086408, WO 04/039762, WO 04/039766, WO 04/045618 and WO 04/046083; A2b antagonists such as those described in WO 02/42298; and beta-2 adrenoceptor agonists such as albuterol (salbutamol), metaproterenol, terbutaline, salmeterol fenoterol, procaterol, and especially, formoterol and pharmaceutically acceptable salts thereof, and compounds (in free or salt or solvate form) of formula I of WO 0075114, which document is incorporated herein by reference, preferably compounds of the Examples thereof, especially a compound of formula

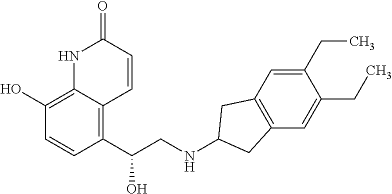

and pharmaceutically acceptable salts thereof, as well as compounds (in free or salt or solvate form) of formula I of WO 04/16601, and also compounds of WO 04/033412. Suitable bronchodilatory drugs include anticholinergic or antimuscarinic compounds, in particular ipratropium bromide, oxitropium bromide, tiotropium salts and CHF 4226 (Chiesi), and glycopyrrolate, but also those described in WO 01/04118, WO 02/51841, WO 02/53564, WO 03/00840, WO 03/87094, WO 04/05285, WO 02/00652, WO 03/53966, EP 424021, U.S. Pat. No. 5,171,744, U.S. Pat. No. 3,714,357, WO 03/33495 and WO 04/018422.

Suitable antihistamine drug substances include cetirizine hydrochloride, acetaminophen, clemastine fumarate, promethazine, loratidine, desloratidine, diphenhydramine and fexofenadine hydrochloride, activastine, astemizole, azelastine, ebastine, epinastine, mizolastine and tefenadine as well as those disclosed in WO 03/099807, WO 04/026841 and JP 2004107299.

Other useful combinations of compounds of the invention with anti-inflammatory drugs are those with antagonists of chemokine receptors, e.g. CCR-1, CCR-2, CCR-3, CCR-4, CCR-5, CCR-6, CCR-7, CCR-8, CCR-9 and CCR10, CXCR1, CXCR2, CXCR3, CXCR4, CXCR5, particularly CCR-5 antagonists such as Schering-Plough antagonists SC-351125, SCH-55700 and SCH-D, Takeda antagonists such as N-[[4-[[[6,7-dihydro-2-(4-methylphenyl)-5H-benzo-cyclohepten-8-yl]carbonynamino]phenyl]-methyl]tetrahydro-N,N-dimethyl-2H-pyran-4-aminium chloride (TAK-770), and CCR-5 antagonists described in U.S. Pat. No. 6,166,037 (particularly claims 18 and 19), WO 00/66558 (particularly claim 8), WO 00/66559 (particularly claim 9), WO 04/018425 and WO 04/026873.

The structure of the active compounds identified by code nos., generic or trade names may be taken from the actual edition of the standard compendium “The Merck Index” or from databases, e.g. Patents International (e.g. IMS World Publications).

The above-mentioned compounds, which can be used in combination with a compound of the formula (I), can be prepared and administered as described in the art, such as in the documents cited above.

By “combination”, there is meant either a fixed combination in one dosage unit form, or a kit of parts for the combined administration where a compound of the formula (I) and a combination partner may be administered independently at the same time or separately within time intervals that especially allow that the combination partners show a cooperative, e.g. synergistic effect.

The invention also provides a pharmaceutical preparation, comprising a compound of formula I as defined herein, or an N-oxide or a tautomer thereof, or a pharmaceutically acceptable salt of such a compound, or a hydrate or solvate thereof, and at least one pharmaceutically acceptable carrier.

A compound of formula I can be administered alone or in combination with one or more other therapeutic compounds, possible combination therapy taking the form of fixed combinations or the administration of a compound of the invention and one or more other therapeutic (including prophylactic) compounds being staggered or given independently of one another, or the combined administration of fixed combinations and one or more other therapeutic compounds. A compound of formula I can besides or in addition be administered especially for tumor therapy in combination with chemotherapy, radiotherapy, immunotherapy, phototherapy, surgical intervention, or a combination of these. Long-term therapy is equally possible as is adjuvant therapy in the context of other treatment strategies, as described above. Other possible treatments are therapy to maintain the patient's status after tumor regression, or even chemopreventive therapy, for example in patients at risk.

The dosage of the active ingredient depends upon a variety of factors including type, species, age, weight, sex and medical condition of the patient; the severity of the condition to be treated; the route of administration; the renal and hepatic function of the patient; and the particular compound employed. A physician, clinician or veterinarian of ordinary skill can readily determine and prescribe the effective amount of the drug required to prevent, counter or arrest the progress of the condition. Optimal precision in achieving concentration of drug within the range that yields efficacy requires a regimen based on the kinetics of the drug's availability to target sites. This involves a consideration of the distribution, equilibrium, and elimination of a drug.

The dose of a compound of the formula I or a pharmaceutically acceptable salt thereof to be administered to warm-blooded animals, for example humans of approximately 70 kg body weight, is preferably from approximately 3 mg to approximately 5 g, more preferably from approximately 10 mg to approximately 1.5 g per person per day, divided preferably into 1 to 3 single doses which may, for example, be of the same size. Usually, children receive half of the adult dose.

The compounds of the invention may be administered by any conventional route, in particular parenterally, for example in the form of injectable solutions or suspensions, enterally, e.g. orally, for example in the form of tablets or capsules, topically, e.g. in the form of lotions, gels, ointments or creams, or in a nasal or a suppository form. Topical administration is e.g. to the skin. A further form of topical administration is to the eye. Pharmaceutical compositions comprising a compound of the invention in association with at least one pharmaceutical acceptable carrier or diluent may be manufactured in conventional manner by mixing with a pharmaceutically acceptable carrier or diluent.

The invention relates also to pharmaceutical compositions comprising an effective amount, especially an amount effective in the treatment of one of the above-mentioned disorders, of a compound of formula I or an N-oxide or a tautomer thereof together with one or more pharmaceutically acceptable carriers that are suitable for topical, enteral, for example oral or rectal, or parenteral administration and that may be inorganic or organic, solid or liquid. There can be used for oral administration especially tablets or gelatin capsules that comprise the active ingredient together with diluents, for example lactose, dextrose, mannitol, and/or glycerol, and/or lubricants and/or polyethylene glycol. Tablets may also comprise binders, for example magnesium aluminum silicate, starches, such as corn, wheat or rice starch, gelatin, methylcellulose, sodium carboxymethylcellulose and/or polyvinylpyrrolidone, and, if desired, disintegrators, for example starches, agar, alginic acid or a salt thereof, such as sodium alginate, and/or effervescent mixtures, or adsorbents, dyes, flavorings and sweeteners. It is also possible to use the pharmacologically active compounds of the present invention in the form of parenterally administrable compositions or in the form of infusion solutions. The pharmaceutical compositions may be sterilized and/or may comprise excipients, for example preservatives, stabilisers, wetting compounds and/or emulsifiers, solubilisers, salts for regulating the osmotic pressure and/or buffers. The present pharmaceutical compositions, which may, if desired, comprise other pharmacologically active substances are prepared in a manner known per se, for example by means of conventional mixing, granulating, confectionning, dissolving or lyophilising processes, and comprise approximately from 1% to 99% by weight, especially from approximately 1% to approximately 60%, active ingredient(s).

Additionally, the present invention provides a compound of formula I or an N-oxide or a tautomer thereof, or a pharmaceutically acceptable salt of such a compound, for use in a method for the treatment of the human or animal body, especially for the treatment of a disease mentioned herein, most especially in a patient requiring such treatment.

The present invention also relates to the use of a compound of formula I or a tautomer thereof, or a pharmaceutically acceptable salt of such a compound, for the preparation of a medicament for the treatment of a proliferative disease, an inflammatory disease, or an obstructtive airway disease, or disorders commonly occurring in connection with transplantation.

Furthermore, the invention relates to a method for the treatment of a proliferative disease which responds to an inhibition of lipid kinases and/or PI3-kinase-related protein kinases, in particular the PI3 kinase, and/or mTOR, and/or DNA protein kinase activity, which comprises administering a compound of formula I or a pharmaceutically acceptable salt thereof, wherein the radicals and symbols have the meanings as defined above, especially in a quantity effective against said disease, to a warm-blooded animal requiring such treatment.

Furthermore, the invention relates to a pharmaceutical composition for treatment of solid or liquid tumours in warm-blooded animals, including humans, comprising an antitumor effective dose of a compound of the formula I as described above or a pharmaceutically acceptable salt of such a compound together with a pharmaceutical carrier.

### Manufacturing Process:

The invention relates also to a process for the manufacture of a compound of the formula I, an N-oxide thereof, a solvate thereof and/or a salt thereof.

Compounds of the formula I can be prepared according to or in analogy to methods that, in principle but with other educts, intermediates and/or final products, are known in the art, especially and according to the invention by a novel process comprising

a) reacting a compound of the formula II,

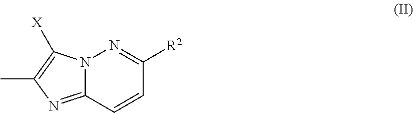

wherein R² is as defined for a compound of the formula I and X is halo, preferably chloro, bromo or iodo, or is trifluoromethansulfonyloxy, under cross-coupling conditions with a boronic acid or boronic acid ester or an organotin compound of the formula III,

R¹-D (III)

wherein R¹ is as defined for a compound of the formula I and is bound via a carbon atom to D and D is —B(OH₂) in free form or in esterified form, e.g. as dialkoxy ester or as a group of the formula A,

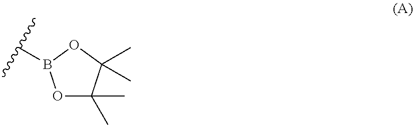

or is —Sn(alk)₃ wherein alk is alkyl, preferably C₁-C₇-alkyl, more preferably methyl,
or
b) reacting a boronic acid or boronic acid ester or organotin compound of the formula IV,

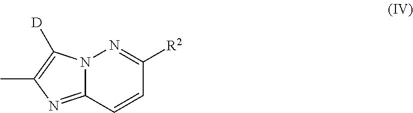

wherein R² is as defined for a compound of the formula I and D is —B(OH₂) in free form or in esterified form, e.g. as a group of the formula A shown under a), or is —Sn(alk)₃ wherein alk is alkyl, preferably C₁-C₇-alkyl, more preferably methyl, under cross-coupling conditions with a compound of the formula V,

R¹—X (V)

wherein R¹ is as defined for a compound of the formula I and X is halogen, especially chloro, bromo or iodo, or trifluoromethansulfonyloxy,
or
c) reacting a compound of the formula VI,

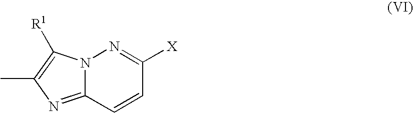

wherein R¹ is as defined for a compound of the formula I and X is halo, especially chloro, bromo or iodo, or is trifluoromethansulfonyloxy, under cross-coupling conditions with a boronic acid or boronic acid ester or organotin compound of the formula VII,

R²-D (VII)

wherein R² is as defined for a compound of the formula I and D is —B(OH₂) in free form or in esterified form, e.g. as a group of the formula A shown under a), or is —Sn(alk)₃ wherein alk is alkyl, preferably C₁-C₇-alkyl, more preferably methyl,
or
d) reacting a pyridazine compound of the formula VIII,

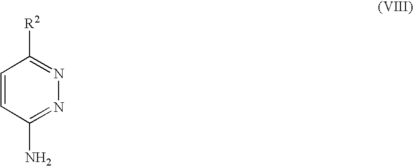

wherein R² is as defined for a compound of the formula I, with a haloketone of the formula IX,

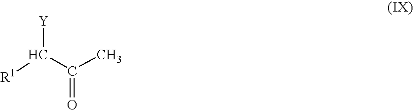

wherein R¹ is as defined for a compound of the formula I and Y is halo, especially chloro or bromo,
or
e) for the manufacture of a compound of the formula I wherein R¹ is pyrazol-3-yl, reacting a compound of the formula X,

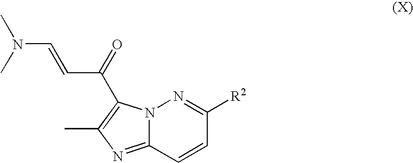

wherein R² is as defined for a compound of the formula I, with hydrazine or a hydrate and/or salt thereof,
and, if desired, a compound of the formula I obtainable according to any one of the reactions a) to e) given above is converted into a different compound of the formula I, an obtainable salt of a compound of the formula I is converted into a different salt thereof, an obtainable free compound of the formula I is converted into a salt thereof, and/or an obtainable isomer of a compound of the formula I is separated from one or more different obtainable isomers of the formula I.

In the following more detailed description of preferred variants of the processes, optional reactions and conversions, synthesis of starting materials and intermediates and the like, R¹ and R² have the meanings given for a compound of the formula I or the compound mentioned specifically, while D is as defined for a compound of the formula III, X as for a compound of the formula II, Y as for a compound of the formula IX, alk as defined for a compound of the formula X, Het as for a compound of the formula XI, Hyl as for a compound of the formula XII and Hea as for a compound of the formula XIII, in each case if not indicated otherwise, respectively.

Where useful or required, the reactions can take place under an inert gas, such as nitrogen or argon. Heating can, for example, be effected by means or microwaves or (e.g. oil) baths or the like, where required in sealed reaction vessels to avoid evaporation at the temperatures used.

The reaction given under process variants a), b) and c), respectively, is, if D is —B(OH)₂ in free form or in esterified form, preferably carried out under the conditions of a Suzuki-reaction or in analogy thereto, preferably in one or more aprotic solvents, such as dimethylformamide (DMF), in an alcohol such as ethanol, in a cyclic ether such as tetrahydrofurane, in a cyclic hydrocarbon such as toluene, a mixture of two or more such solvents and optionally water in the presence of a catalyst for the cross-coupling, especially a noble metal catalyst, preferably a palladium catalyst, such as palladium(II) complex, for example bis(triphenylphosphine)palladium (II) dichloride or [1,1′-Bis(diphenylphosphino)ferrocene]dichloropalladium(II), in the presence of a base, such as potassium carbonate, an alkalimetal C₁-C₇-alkanoate, such as sodium or potassium acetate, sodium hydroxide or sodium carbonate, at a preferred temperature in the range from 80° C. to 150° C.; or according to a another preferred method in a cyclic ether solvent, e.g. tetrahydrofurane, in the presence of a catalyst for the cross coupling, especially a noble metal catalyst, preferably a palladium (0) complex, for example tris(dibenzylideneacetone)-dipalladium(0), or of palladium dibenzylideneacetone as precursor, where useful in the presence an appropriate ligand, such as 2-dicyclohexylphosphino-2′,6′-dimethoxybiphenyl (SPhos) or 2-dicyclohexylphosphino-2′-(N,N-dimethylamino)-biphenyl (P1), and in the presence of a base, e.g. as mentioned above or potassium phosphate, and at a preferred temperatures in the range from 80 to 150° C.; if required conducting the reaction in a sealed vessel (e.g. a seal reactor or a microwave vessel) if the boiling point of the reaction mixture is exceeded and/or especially if (as is a preferred embodiment) the heating is effected by microwave excitation. Where required, other or additional catalyst(s) can be added, e.g. (PdCl₂(PPh₂).Fe.CH₂Cl₂), or mixtures of catalysts can be used.

The reaction given under process variants a), b) and c), respectively, is, if D is —Sn(alk)₃ wherein alk is alkyl, preferably C₁-C₇-alkyl, more preferably methyl, is preferably conducted under Stille coupling conditions, or in analogy thereto, preferably in an appropriate polar solvent, such as N,N-dimethylacetamide or N,N-dimethylformamide, an ether, such as tetrahydrofurane, and/or a mixture of two or more such solvents, in the presence of a a palladium catalyst, especially a palladium (0) complex, for example tetrakistriphenyl-palladium, e.g. at temperatures in the range from 80 to 160° C., if required conducting the reaction in a sealed vessel (e.g. a seal reactor or a microwave vessel) if the boiling point of the reaction mixture is exceeded and/or especially if (as is a preferred embodiment) the heating is effected by microwave excitation.

The reaction between a compound of the formula VIII and a compound of the formula IX (reaction variant d) above) preferably takes place in an appropriate solvent, such as an alcohol, for example in ethanol, at elevated temperatures, e.g. in the range from 80 to 180° C., e.g. at 100 to 170° C., in the absence or if useful presence of a tertiary nitrogen base, such as a tri-(lower alkyl)-amine, for example triethylamine.

The reaction (ring formation) between a compound of the formula X and hydrazine, a salt and/or a solvate thereof, preferably takes place e.g. in an appropriate polar solvent, such as an alcohol, e.g. ethanol, for example at elevated temperatures, e.g. in the range from 50 to 140° C.

Where temperatures are givent hereinbefore or hereinafter, “about” has to be added, as minor deviations from the numeric values given, e.g. variations of ±10%, are tolerable.

### Protecting Groups

If one or more other functional groups, for example carboxy, hydroxy, amino, or mercapto, are or need to be protected in a starting material, e.g. in any one or more starting materials of the formula II or III or other starting materials, intermediates and educts mentioned below, because they should not take part in the reaction or disturb the reaction, these are such groups as are usually used in the synthesis of peptide compounds, and also of cephalosporins and penicillins, as well as nucleic acid derivatives and sugars. Protecting groups are such groups that are no longer present in the final compounds once they are removed, while groups that remain as substitutents are not protecting groups in the sense used here which is groups that are added at a certain intermediate stage and removed to obtain a final compound. For example, tert-butoxy if remaining in a compound of the formula I is a substituent, while if it is removed to obtain the final compound of the formula I it is a protecting group.

The protecting groups may already be present in precursors and should protect the functional groups concerned against unwanted secondary reactions, such as acylations, etherifications, esterifications, oxidations, solvolysis, and similar reactions. It is a characteristic of protecting groups that they lend themselves readily, i.e. without undesired secondary reactions, to removal, typically by acetolysis, protonolysis, solvolysis, reduction, photolysis or also by enzyme activity, for example under conditions analogous to physiological conditions, and that they are not present in the end-products. The specialist knows, or can easily establish, which protecting groups are suitable with the reactions mentioned above and below.

The protection of such functional groups by such protecting groups, the protecting groups themselves, and their removal reactions are described for example in standard reference works, such as J. F. W. McOmie, “Protective Groups in Organic Chemistry”, Plenum Press, London and New York 1973, in T. W. Greene, “Protective Groups in Organic Synthesis”, Third edition, Wiley, New York 1999, in “The Peptides”; Volume 3 (editors: E. Gross and J. Meienhofer), Academic Press, London and New York 1981, in “Methoden der organischen Chemie” (Methods of organic chemistry), Houben Weyl, 4th edition, Volume 15/I, Georg Thieme Verlag, Stuttgart 1974, in H.-D. Jakubke and H. Jescheit, “Aminosauren, Peptide, Proteine” (Amino acids, peptides, proteins), Verlag Chemie, Weinheim, Deerfield Beach, and Basel 1982, and in Jochen Lehmann, “Chemie der Kohlenhydrate: Monosaccharide and Derivate” (Chemistry of carbohydrates: monosaccharides and derivatives), Georg Thieme Verlag, Stuttgart 1974.

An example for an amino (or imino) protecting group is tert-butoxycarbonyl which can be introduced used to protect amino or imino groups and can be removed e.g. by hydrolysis, e.g. with an acid, such as trifluoroacetic acid or hydrochloric acid, in an appropriate solvent, e.g. methylene chloride or dioxane, e.g. at temperatures in the range from 0 to 50° C.

### Optional Reactions and Conversions

A compound of the formula I may be converted into a different compounds of the formula I according to standard reaction procedures.

For example, in a compound of the formula I wherein R¹ is heteroaryl (meaning unsaturated heterocyclyl), such as pyridyl (=pyridinyl), that is substituted by halo, especially by chloro or bromo, e.g. in the p-position, the halo can be replaced by a unsubstituted or substituted ring nitrogen comprising unsaturated heterocyclyl bound via a ring nitrogen atom by reaction with a compound of the formula XI,

H-Het (XI)

wherein Het is an unsubstituted or substituted unsaturated heterocyclyl moiety bound to the hydrogen via a ring nitrogen atom, such as 1,2,4-triazol, pyrazole, benzimidazole, 3-trifluoromethyl-pyrazol, under Ullman-type reaction conditions, e.g. as in see e.g. Chem. Eur. J. (2004), 10, 5607 on the general Ullmann-type arylation of nucleophiles, preferably by reacting the corresponding compound of the formula I and the compound of the formula XI in the presence of Cu₂O, a ligand such as salicylaldehyde hydrazone, a base such as caesium carbonate and a solvent such as acetonitrile at preferred temperatures in the range from 100 to 180° C., e.g. at 160 to 150° C., for example in a microwave oven. This leads to a compound of the formula I wherein R¹ is heteroaryl, e.g. phenyl, substituted by unsubstituted or substituted ring nitrogen comprising unsaturated heterocyclyl bound via a ring nitrogen atom.

Alternatively, for example, in a compound of the formula I wherein R¹ is heteroaryl, such as pyridyl, that is substituted by halo, especially by chloro or bromo, e.g. in the p-position, the halo can be replaced by an unsubstituted or substituted saturated heterocyclyl comprising a nitrogen atom by reaction with a compound of the formula XII,

H-Hyl (XII)

wherein Hyl is an unsubstituted or substituted saturated heterocyclyl moiety bound to the hydrogen via a ring nitrogen atom, such as valerolactame, morpholine, 2-pyrrolidinone or N-methylpiperazine, under reaction conditions such as those described in the reference mentioned in Example 14, e.g. reacting the heterocyclic compound of the formula XI and the corresponding compound of the formula I in the presence of CuI, a base, such as potassium carbonate, and of proline in an appropriate solvent, such as dimethylsulfoxide, preferably at temperatures in the range from 80 to 130° C.

Yet alternatively, in a compound of the formula I wherein R¹ is heteroaryl, such as phenyl, that is substituted by halo, especially by chloro or bromo, e.g. in the p-position, the halo can be replaced by an unsubstituted or substituted saturated heterocyclyl bound via a ring carbon atom by reaction with a compound of the formula XIII,

D*-Hea (XIII)

wherein Hea is unsaturated heterocyclyl (heteroaryl) and D* has the meaning of D given above for compounds of the formulae III, IV and VII, by reaction under conditions analogous to those mentioned above for reaction variants a), b) and c).

In the preceding and subsequent paragraphs on conversions, heterocyclyl or heteroaryl Het, Hyl and Hea can be unsubstituted or substituted as described above for unsubstituted or substituted heterocyclyl, preferably by substitutents other than halo.

In a compound of the formula I wherein an amino or imino group carries a C₁-C₇-alkoxycarbonyl, such as tert-butoxycarbonyl group, this group may be removed under conditions analogous to those described above unter “Protecting groups”.

In a compound of the formula I wherein R¹ is heterocyclyl (especially unsaturated hetero-cyclyl=heteroaryl, e.g. pyrazolyl, pyrazinyl or pyridyl) carrying a hydroxy group, the hydroxy group can be converted into halo, e.g. chloro, by reaction, e.g. with an inorganic acid halide, such as phosphorus oxychloride, under customary conditions, e.g. in the absence or presence of a solvent at elevated temperatures, such as reflux temperature.

In a compound of the formula I wherein R¹ is heterocyclyl comprising an imino group (that is, —NH—), e.g. in pyrazol-3-yl or pyrazin-2-yl, the hydrogen in the imino group may be acylated to C₁-C₇-alkanoylimino, unsubstituted or substited benzoylimino, C₁-C₇-alkanesulfonylimino or unsubstituted or substituted benzenesulfonylino, by reaction with a corresponding acid halogenide, e.g. acid chloride, or with the help of an in situ activating agent (coupling agent), such as HATU or HBTU or the like, see e.g. below for further coupling agents and conditions, under customary reaction conditions, e.g. in the presence of a solvent, such as tetrahydrofurane, or in its absence, in the presence of a tertiary nitrogen base, such as pyridine or triethylamine, at temperatures e.g. in the range from 0 to 50° C.

In a compound of the formula I wherein R² carries an C₁-C₇-alkoxycarbonylamino-C₁-C₇-alkoxy substituent, this may be converted to the free amino-C₁-C₇-alkoxy substituent e.g. as described above for the deprotection of C₁-C₇-alkoxycarbonylamino to amino.

In a compound of the formula I wherein R² carries an amino-C₁-C₇-alkoxy substituent, this substitutent can be converted into C₆-C₁₄-arylcarbonylamino-C₂-C₇-alkoxy wherein C₆-C₁₄-aryl is unsubstituted or substituted by one or more substituents independently selected from the group consisting of C₁-C₇-alkyl, hydroxy, C₁-C₇-alkoxy and halo, or into heterocyclylcarbonylamino-C₁-C₇-alkoxy wherein heterocyclyl has 3 to 10 ring atoms and has one or more hetero ring atoms selected from O, S and N, especially N, by reaction with a corresponding acid or a reactive acid derivative (such as acid halogenide, e.g. acid chloride) which can also be formed in situ, e.g. by means of a coupling agent that forms a reactive derivative of the carboxyl group in situ, for example dicyclohexylcarbodiimide/1-hydroxybenzotriazole (DCC/HOBT); bis(2-oxo-3-oxazolidinyl)phosphinic chloride (BOPCl); O-(1,2-dihydro-2-oxo-1-pyridyl)-N,N,N′,N′-tetramethyluronium tetrafluoroborate (TPTU); O-benzotriazol-1-yl)-N,N,N′,N′-tetramethyluronium tetrafluoroborate (TBTU); (benzotriazol-1-yloxy)-tripyrrolidinophosphonium-hexafluorophosphate (PyBOP), O-(1H-6-chlorobenzotriazole-1-yl)-1,1,3,3-tetramethyluronium hexafluorophosphate, 1-(3-dimethylaminopropyl)-3-ethylcarbodiimide hydrochloride/hydroxybenzotriazole, O-(7-azabenzotriazol-1-yl)-N,N,N′,N′-tetramethyluronium-hexafluorophosphat (HATU) or/1-hydroxy-7-azabenzotriazole (EDC/HOBT or EDC/HOAt) or HOAt alone, or with (1-chloro-2-methyl-propenyl)-dimethylamine. For review of some other possible coupling agents, see e.g. Klauser; Bodansky, Synthesis (1972), 453-463. The reaction mixture, which advantageously can comprise an appropriate solvent, e.g. dimethyl formamide or dioxane, and/or N-methylmorpholine, is preferably kept, e.g. stirred, at a temperature of between approximately −20 and 80° C., especially between 0° C. and 60° C., e.g. at room temperature or at about 50° C.

In a compound of the formula I wherein R² carries an amino-C₁-C₇-alkoxy substituent, this substitutent can be converted into C₆-C₁₄-arylaminocarbonylamino-C₂-C₇-alkoxy (C₆-C₁₄-aryl-NH—C(═O)—NH—C₂-C₇-alkoxy) wherein C₆-C₁₄-aryl is defined as above, preferably is phenyl or naphthyl, and is in each case unsubstituted or substituted by one or more, especially up to three, substituents independently selected from the group consisting of C₁-C₇-alkyl, especially methyl or ethyl, halo-C₁-C₇-alkyl, especially trifluoromethyl, hydroxy, C₁-C₇-alkoxy, especially methoxy, and halo, especially fluoro, or into heterocyclylaminocarbonylamino-C₁-C₇-alkoxy wherein heterocyclyl has 3 to 10 ring atoms and has one or more hetero ring atoms selected from O, S and N, especially N, by reaction with a corresponding isocyanate under customary conditions.

A compound of the formula I wherein R¹ is heterocyclyl, such as pyridyl, that is substituted by cyano can be converted to a corresponding compound of the formula I wherein instead of the cyano an 1H-tetrazol-5-yl moiety is present by reaction with an azide salt, such as sodium azide, preferably in the presence of an ammonium salt, such as ammonium chloride, at a temperature e.g. from 120 to 160° C.

A compound of the formula I wherein R¹ is heterocyclyl, such as pyrazolyl, pyrazinyl or pyridyl, substituted by nitro can be reduced to a corresponding compound of the formula I wherein instead of the nitro an amino group is present, e.g. by reduction by hydrogenation in the presence of a hydrogenation catalyst, e.g. a noble metal catalyst, such as palladium, which can preferably be bound to a carrier, such as charcoal, in an appropriate solvent, such as an alcohol, e.g. methanol, preferably at temperatures in the range from 0 to 50° C., e.g. at room temperature. As by-product, the alkylation product resulting from the alcohol can be obtained, e.g. in the case of methanol the corresponding methylamino compound of the formula I, which can be isolated according to standard procedures, such as chromatography.

In a compound of the formula I wherein R¹ is heteroaryl, such as pyrazolyl, pyrazinyl or pyridyl, substituted by chloro, bromo or iodo, the chloro, bromo or iodo can be converted into a group D as described above for a compound of the formula III, for example by reaction first with n-butyllithium (replacing the chloro, bromo or iodo by Li) and subsequent reaction with a corresponding trialkoxyborane, such as triisopropylborane; or by reaction of the chloro, bromo or iodo compound in the presence of a transition metal catalyst (e.g. PdCl(dppf) with alkoxydiborone), or the like. Alternatively, also triflate (trifluoromethanesulfonyloxy) substituents instead of halo can be substituted accordingly in corresponding starting materials. The free boronic acids (unesterified) can be obtained e.g. by working up in the presence of an inorganic acid, such as hydrochloric acid.

The compound of the formula I carrying a group D as just described can then be reacted with an unsubstituted or substituted aryl or unsaturated heterocyclyl compound under conditions as described above for reaction a) (e.g. cross coupling, such as Suzuki coupling) to a corresponding compound of the formula I wherein instead of the original chloro, bromo or iodo an aryl or unsaturated heterocyclyl substituent is present (each of which may be substituted as well as described above).

A nitrogen ring atom of the imidazo[1,2-b]pyridazine core or a nitrogen-containing heterocyclyl substituent can form an N-oxide in the presence of a suitable oxidizing agent, e.g. a peroxide, such as m-chloro-perbenzoic acid or hydrogen peroxide.

Also in the optional process steps, carried out “if desired”, functional groups of the starting compounds which should not take part in the reaction may be present in unprotected form or may be protected for example by one or more of the protecting groups mentioned hereinabove under “protecting groups”. The protecting groups are then wholly or partly removed according to one of the methods described there.

Salts of a compound of formula I with a salt-forming group may be prepared in a manner known per se. Acid addition salts of compounds of formula I may thus be obtained by treatment with an acid or with a suitable anion exchange reagent, salt with bases by treatment with a corresponding base or a suitable cation exchange reagent.

Salts can usually be converted to free compounds, e.g. acid addition salts by treating with suitable basic compounds, for example with alkali metal carbonates, alkali metal hydrogen-carbonates, or alkali metal hydroxides, typically potassium carbonate or sodium hydroxide, salt with bases by treating with suitable acid compounds, such as hydrochloric acid, sulfuric acid or the like.

Mixtures of constitutional isomers or of products and by-products can be separated according to standard procedures, e.g. by distribution, chromatography or the like.

Stereoisomeric mixtures, e.g. mixtures of diastereomers, can be separated into their corresponding isomers in a manner known per se by means of suitable separation methods. Diastereomeric mixtures for example may be separated into their individual diastereomers by means of fractionated crystallization, chromatography, solvent distribution, and similar procedures. This separation may take place either at the level of a starting compound or in a compound of formula I itself. Enantiomers may be separated through the formation of diastereomeric salts, for example by salt formation with an enantiomer-pure chiral acid, or by means of chromatography, for example by HPLC, using chromatographic substrates with chiral ligands. Separation may take place in solutions and/or in emulsions, e.g. macro- or microemulsions.

It should be emphasized that reactions analogous to the conversions mentioned in this chapter may also take place at the level of appropriate intermediates (and are thus useful in the preparation of corresponding starting materials).

### Starting Materials:

The starting materials of the formulae II, III, IV, V, VI, VII, VIII, IX, X, XI, XII and XIII as well as other starting materials, intermediates or educts mentioned herein, e.g. below, can be prepared according to or in analogy to methods that are known in the art, the materials are known in the art and/or are commercially available, or by or in analogy to methods mentioned in the Examples. Novel starting materials (e.g. in Example 1 the compound of Stage 1.1, 3-bromo-6-(3,4-dimethoxy-phenyl)-2-methyl-imidazo[1,2-b]pyridazine, or analogues wherein instead of the bromo a chloro or iodo or trifluoromethansulfonyloxy is present), as well as processes for the preparation thereof, are likewise an embodiment of the present invention. In the preferred embodiments, such starting materials are used and the reaction chosen are selected so as to enable the preferred compounds to be obtained.

Starting materials of the formula II are known in the art, commercially available or can be prepared according to or in analogy to methods known in the art.

For example, a compound of the formula II can be obtained by reacting a compound of the formula XIV,

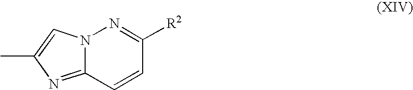

in the presence of a halogenating agent, e.g. N-iodo-, N-bromo- or N-chloro-succinimide (with N-bromosuccinimide being preferred), in an appropriate solvent, such as an alkylated amide, e.g. dimethyl formamide, or a halogenide, methylene chloride, chloroform or the like, e.g. at temperatures in the range from −20 to 50° C., to the corresponding compound of the formula (II) wherein X is halo (preferably bromo).

In a compound of the formula XIV wherein R² has a hydroxy substituent, this hydroxy can be converted into C₁-C₇-alkoxy e.g. by reaction in the presence of a base, such as potassium carbonate, with a corresponding C₁-C₇-alkylhalogenide, such as -iodide, in an appropriate solvent, e.g. N,N-dimethylacetamide or the like, at elevated temperatures, e.g. in the range from 50° C. to 120 !C, e.g. at 100° C.

A compound of the formula XIV can, for example, be obtained by reacting a compound of the formula VIII with a halogenated acetone of the formula XV,

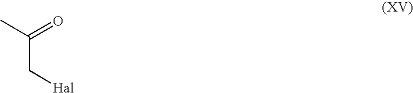

wherein Hal is halo, especially chloro, under conditions analogous to those described above (under process variant d)) for the reaction of a compound of the formula VIII with a halo-ketone compound of the formula IX.

A compound of the formula VIII can, for example, be obtained by reacting a pyridazine compound of the formula XVI,

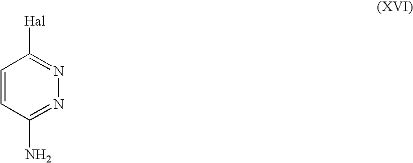

wherein Hal is halo, especially chloro or bromo, with a boronic acid or boronic acid ester of the formula VII mentioned above under conditions analogous to those mentioned above (for process variant c)) for the reaction of a compound of the formula VI and a compound of the formula VII.

Alternatively, a compound of the formula XIV can be obtained by reacting a compound of the formula XX,

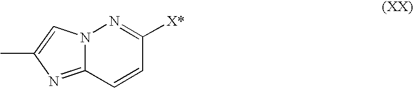

wherein X has the same meaning as X in formula II or VI, with a compound of the formula VII given above under conditions as described for process c) above.

A compound of the formula XX can, for example, be obtained by reacting a pyridazine compound of the formula XVI as described above with a compound of the formula XV as described above, preferably under conditions analogous to those described above (under process variant d)) for the reaction of a compound of the formula VIII with a haloketone compound of the formula IX.

A compound of the formula IV can for example be obtained from a compound of the formula II by replacing the group X with a group —B(OH)₂ in free (obtainable in the presence of an acid, such as hydrochloric acid, from an esterified form) or esterified form e.g. under reaction conditions analogous to those mentioned under the conversions for a compound of the formula I wherein R¹ is unsaturated heterocyclyl (=heteroaryl), such as pyrazolyl, pyrazinyl or pyridyl, substituted by chloro, bromo or iodo, the chloro, bromo or iodo, into the corresponding compound wherein the chloro, bromo or iodo is replaced with a group —B(OH)₂ in free or preferably esterified form; or with a group —Sn(alk)₃ wherein alk is as defined above for a compound of the formula III, IV or VII by reaction with a bis(trialkylstannane), such as bis(tributylstannane) or bis(trimethylstannane), in an appropriate solvent, such as toluene, preferably at elevated temperatures, e.g. from 100° C. to 150° C.

A compound of the formula VI can preferably be obtained by reaction of a compound of the formula XV mentioned above with a compound of the formula IX as defined under process variant d) under reaction conditions analogous to those mentioned above (for process variant d)) for the reaction of a compound of the formula VIII with a compound of the formula IX.

A compound of the formula IX can, for example, be prepared by reacting a compound of the formula XVII,

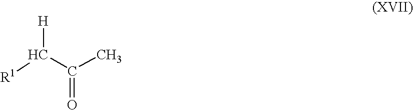

in the presence of a halogenating agent, e.g. an inorganic acid halide, such as a sulfuryl halogenide, preferably sulfurylchloride, in an appropriate solvent, e.g. methylene chloride, e.g. at temperatures in the range from −20 to 50° C.

A compound of the formula XVII can, for example, be obtained by reacting a compound of the formula XVIII,

R¹—Br (XVIII)

with isopentyl acetate in the presence of tributyltin methoxide, a catalyst, e.g. Pd₂(dba)₃, and of 2-dicyclohexylphosphino-2′-(N,N-dimethylamino)-biphenyl in an appropriate solvent, e.g. toluene, at elevated temperatures, e.g. under reflux conditions.

Alternatively, a compound of the formula XVII can be obtained by reacting an aldehyde of the formula XIX,

R¹—CHO (XIX)

in the presence of nitroethane and ammonium acetate at an elevated temperature, e.g. 60 to 130° C., followed by conversion of the resulting 2-nitropropenyl intermediate in the presence of iron powder (with or without addition of FeCl₃) and an acid, such as hydrogen chloride or acetic acid, in an aqueous solvent, e.g. at elevated temperature, for example at temperatures between 50° C. and reflux temperature of the reaction mixture.

A compound of the formula III can, for example, be obtained by reacting a compound of the formula XXI,

R²—X* (XXI)

wherein R² is as defined for a compound of the formula I and X* is defined as X in compounds of the formula II, V or VI, by replacing the group X* with a group —B(OH)₂ in free (obtainable in the presence of an acid, such as hydrochloric acid, from an esterified form) or esterified form e.g. under reaction conditions analogous to those mentioned under the conversions for a compound of the formula I wherein R¹ is unsaturated heterocyclyl (=heteroaryl), such as pyrazolyl, pyrazinyl or pyridyl, substituted by chloro, bromo or iodo, the chloro, bromo or iodo, into the corresponding compound wherein the chloro, bromo or iodo is replaced with a group —B(OH)₂ in free or preferably esterified form; or with a group —Sn(alk)₃ wherein alk is as defined above for a compound of the formula III, IV or VII by reaction with a bis(trialkylstannane), such as bis(tributylstannane) or bis(trimethylstannane), in an appropriate solvent, such as toluene, preferably at elevated temperatures, e.g. from 100° C. to 150° C.

A compound of the formula XXI can, for example, be obtained from a compound of the formula XXII,

R²—H (XXII)

wherein R² is as described for a compound of the formula I by reaction with a N-halo-succinimide, e.g. N-bromo- or N-chloro-succinimide, e.g. in acetonitrile at about room temperature, or the like.

Compound of the formula V can, in analogy to the preceding paragraph, be prepared from corresponding compounds of the formula XXIII,

R¹—H (XXIII)

wherein R¹ is as described for a compound of the formula I.

A compound of the formula X can, for example, be prepared starting from a compound of the formula XXIV,

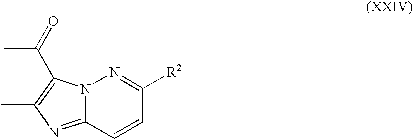

wherein R² is as defined for a compound of the formula I, by reaction with dimethylformamide dimethylacetale, e.g. in the absence of a solvent at elevated reaction temperatures, e.g. in the range from 100 to 150° C.

A compound of the formula XXIV can, for example, be obtained by reaction of a compound of the formula VIII, as defined above, with 3-chloro-2,4-pentanedione, e.g. in an alcohol, such as ethanol, preferably at elevated temperatures e.g. in the range from 100 to 160° C.

All remaining starting materials, including other starting materials of the formulae for which ways of synthesis are described above, are known, capable of being prepared according to known processes, and/or they are commercially obtainable; in particular, they can be prepared using processes as described or in analogy to those described in the Examples.

### EXAMPLES

The following examples illustrate the invention without limiting the scope thereof.

Temperatures are given in degree Celsius (° C.). Where no temperature is given, the reaction is at room temperature. Ratios of solvents or eluents are given as volume per volume (v/v).

Note that some compounds of the formula I are also presented as intermediates (“Stage . . . ”) below—these compounds that fall under formula I are also to be considered as examples.

### ABBREVIATIONS

ABCR ABCR GmbH & Co. KG, Karlsruhe, Germany
Acros Acros Organics, Geel, Belgium
Aldrich Sigma-Aldrich Corp., St. Louis, Mo., USA
Alfa Aesar ALFA AESAR, Ward Hill, Mass., USA
Boc tert-butoxycarbonyl
Boron Molecular Boron Molecular, Inc., Researcz Triangle Park, N.C., USA
CH2Cl2 dichloromethane
CH3CN acetonitrile
Combi Blocks Combi-Blocks, Inc., San Diego, Calif., USA
DMA N,N-dimethylacetamide
DMF N,N′ dimethyl formamide
DMSO dimethylsulfoxide
Emrys Optimizer Emrys™ Optimizer, Microwave oven from Personal Chemistry, Biotage AB, Uppsala, Sweden
EtOH ethanol
EtOAc ethyl acetate
Fluke Fluke, Buchs, Switzerland (belongs to Sigma-Aldrich)
Fluorochem Fluorochem Ltd., Old Glossop, Derbyshire, United Kingdom
Frontier Frontier Scientific, Inc., Logan, Utah, USA
h hour(s)
HATU O-(7-Azabenzotriazol-1-yl)-N,N,N′,N′-tetramethyluronium hexafluorophosphate
HBTU O-Benzotriazole-N,N,N′,N′-tetramthyl-uronium-hexafluoro-phosphate
HPLC High Performance Liquid Chromatography
hyflo Hyflo Super Cel is a diatomaceous earth used in filtration processes (trademark of Johns Manville Corp., Denver, Colo., USA
K2CO3 potassium carbonate
KOAc potassium acetate
K3PO4 potassium phosphate
Maybridge Maybridge, Trevillett and Tintagel, United Kingdom (belong to Thermo Fischer Scientific, Inc., Waltham, Mass., USA)
MeOH methanol
mL milliliter(s)
min minute(s)
MS mass spectrometry
MS-ES electrospray mass spectrometry
NaHCO3 sodium hydrogencarbonate
Na2CO3 disodium carbonate
Na2SO4 sodium sulfate
NBS N-bromosuccinimide
NEt3 triethylamine
NH3 ammonia
NH4OH ammonium hydroxide
NMP 1-methyl-2-pyrrolidinone
PdCl2(dppf) [1,1′-Bis(diphenylphosphino)ferrocene] dichloropalladium(II)
Pd(dba)₂ palladium dibenzylidenacetone
Pd2(dba)₃ palladium trisdibenzylidenacetone
Pd(PPh3)2Cl2 Bis(triphenylphosphine)palladium(II) chloride
P1 2-dicyclohexylphosphino-2′-(N,N-dimethylamino)-biphenyl
POCl3 phosphorus oxychloride
RT room temperature
sat. saturated
Sigma-Aldrich Sigma-Aldrich Co., St. Louis, Mo., USA
SPhos 2-Dicyclohexylphosphino-2′,6′-dimethoxybiphenyl
TBME tert-butyl methyl ether
TFA trifluoro acetic acid
THF tetrahydrofuran
TLC thin layer chromatography
t_{R} retention time
UV Ultraviolet

### Analytical HPLC Conditions:

### System 1

Linear gradient 20-100% CH₃CN (0.1% TFA) and H₂O (0.1% TFA) in 7 min+2 min 100% CH₃CN (0.1% TFA); detection at 215 nm, flow rate 1 mL/min at 30° C. Column: Nucleosil 100-3 C18HD (125×4 mm)

All HPLC retention times refer to System 1 unless noted otherwise.

### Example 1

### 6-(3,4-Dimethoxy-phenyl)-3-(6-fluoro-pyridin-3-yl)-2-methyl-imidazo[1,2-b]pyridazine (1)

In a 3 ml vial for microwave with crown cap and magnetic stir bar, 80 mg (0.207 mmol) of 3-bromo-6-(3,4-dimethoxy-phenyl)-2-methyl-imidazo[1,2-b]pyridazine (for preparation see Stage 1.1.) and 54 mg (0.204 mmol) of 2-fluoro-5-trimethylstannanyl-pyridine (for preparation see J. Med. Chem. (2004), 47, 2453; instead of the 5-iodo-2-fluoro pyridine as reported in the literature, the commercially available 5-bromo-2-fluoro pyridine (Aldrich) is used) are dissolved in 1.5 ml of DMA. Through the solution a slow flow of Argon gas is passed for 5 min. Thereafter 11.9 mg of tetrakis-triphenylphosphin palladium is added and the reaction mixture is heated in the microwave for 60 min at 150° C. After that time, no more starting material can be detected in the HPLC or MS. The solvent is evaporated, the residue is dissolved acetonitrile and washed three times with hexane. The acetonitrile solution is evaporated and the residue purified by chromatography on silicagel C18. Solvent system: acetonitrile-water, 1% trifluoroacetic acid) to yield the title compound. MS-ES: (M+1)=365.2, HPLC: t_{R}=4.293 min.-

### Stage 1.1: 3-Bromo-6-(3,4-dimethoxy-phenyl)-2-methyl-imidazo[1,2-b]pyridazine

To an ice-cold solution of 300 mg (1.06 mmol) of 6-(3,4-dimethoxy-phenyl)-2-methyl-imidazo[1,2-b]pyridazine in 3 ml of DMF, 178 mg (1.02 mmol) of NBS is added. Stirring is continued for 2 h at 0-5° C. and then 1 additional h at RT. After that time, the reaction mixture is evaporated to dryness, the residue is dissolved in ethylacetate and the organic phase is washed with water (2×) and brine (1×). After drying over Na₂SO₄, the solvent is evaporated. The title compound crystallizes from an ethylacetate-hexane mixture. MS-ES.: 348/350. Rf (CH₂Cl₂-MeOH=95:5)=0.56.

### Stage 1.2: 6-(3,4-Dimethoxy-phenyl)-2-methyl-imidazo[1,2-b]pyridazine

In a 6 ml vial for microwave with crown cap and magnetic stir bar, a mixture of 500 mg (2.05 mmol) 6-(3,4-dimethoxy-phenyl)-pyridazin-3-ylamine, 0.364 ml (4.11 mmol) of chloroacetone (Fluka), and 0.716 ml of Et₃N in 4 ml of ethanol are heated in a microwave (Emrys Optimizer) at 170° C. for 30 min. The reaction mixture is evaporated to dryness and the residue is dissolved in CH₂Cl₂. The organic phase is washed with water (2×) and brine (1×). After drying over Na₂SO₄, the solvent is evaporated and the residue purified by chromatography on silica gel. Solvent system: CH₂Cl₂ (100%; start) to CH₂Cl₂-MeOH 98:2 (end). MS: (M+1)=270; HPLC: t_{R}=3.37 min.

### Example 2

### 6-(3,4-Dimethoxy-phenyl)-2-methyl-3-pyridin-3-yl-imidazo[1,2-b]pyridazine (2)

In a 6 ml vial for microwave with crown cap and magnetic stir bar, a mixture of 50 mg (0.144 mmol) of 3-bromo-6-(3,4-dimethoxy-phenyl)-2-methyl-imidazo[1,2-b]pyridazine (for preparation see Stage 1.1.), 29 mg (0.192 mmol) of 3-pyridylboronic acid dimethyl ester (Apollo Scientific), 80 mg (0.579 mmol) of K2CO3, 6 mg PdCl2(dppf) (ABCR) are suspended in 2 ml of EtOH and 3 ml of toluene (degassing with argon before catalysator is added) and heated in a microwave oven at 110° C. for 26½ h. The reaction mixture is poured into CH2Cl2 and the organic phase is washed with water. After drying with Na2SO4, the solvent is evaporated and the residue purified by chromatography on silicagel. Solvent system: CH2Cl2-EtOAc-MeOH=100:0:0 to 50:40:10. The title compound is isolated as a beige solid. MS-ES: (M+1)=346, HPLC: t_{R}=2.982 min.-

The Example compounds in the following table are prepared in analogy to the compound prepared in Example 2:

| | | | micro- |
|---|---|---|---|
| | | | wave |
| Ex- | | | con- |
| am- | | boronic | ditions; |
| ple | Product | acid | data |
| 3 | 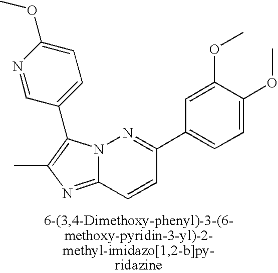 | 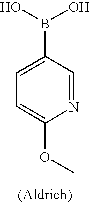 | 110° C. 30 min LC-MS: (M + 1) = 377, HPLC: t_{R} = 4.457 min.- |
| 4 | 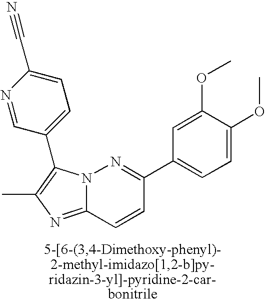 | 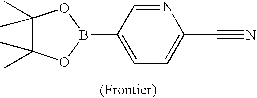 | 110° C. 30 min. LC-MS: (M + 1) = 372, HPLC: t_{R} = 4.401 min.- |
| 5 | 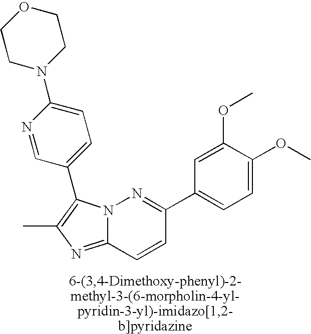 | 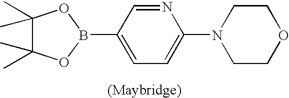 | 105° C. 4½ h LC-MS: (M + 1) = 432, HPLC: t_{R} = 3.350 min.- |
| 6 | 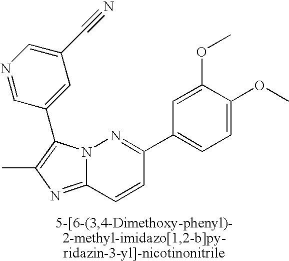 | 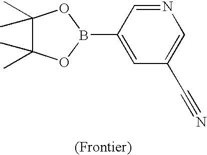 | 105° C. 3½ h LC-MS: (M + 1) = 372, HPLC: t_{R} = 4.112 min.- |
| 7 | 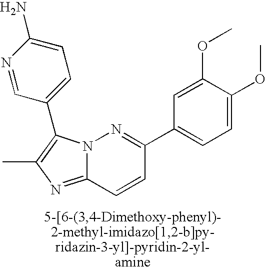 | 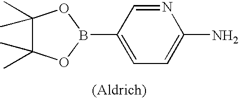 | 110° C. 30 min ES-MS: (M + 1) = 362, HPLC: t_{R} = 2.673 min.- |
| 8 | 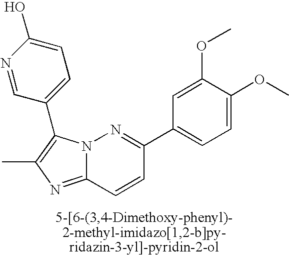 | 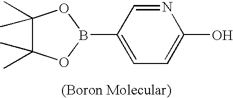 | 105° C. 6½ h ES-MS: (M + 1) = 363, HPLC: t_{R} = 2.949 min.- |
| 9 | 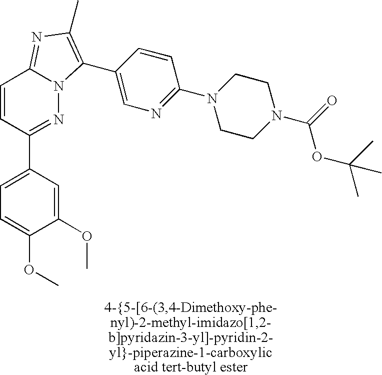 | 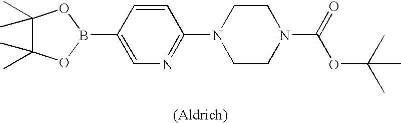 | 110° C. 30 min ES-MS: (M + 1) = 531, HPLC: t_{R} = 4.6 min.- |

### Example 10

### 3-(6-Chloro-pyridin-3-yl)-6-(3,4-dimethoxy-phenyl)-2-methyl-imidazo[1,2-b]pyridazine (10)

32.5 mg (0.0897 mmol) of 5-[6-(3,4-dimethoxy-phenyl)-2-methyl-imidazo[1,2-b]pyridazin-3-yl]-pyridin-2-ol (Ex. 8) in 0.082 ml of POCl3 are heated at reflux temperature for 15 h. After that time, the mixture is cooled and poured into ice. After extracting the water phase with CH2Cl2 and after washing the organic phase with water, the solvent is dried over Na2SO4 and evaporated to yield the title compound. LC-ES: (M+1)=381; HPLC: t_{R}=4.568 min.-

### Example 11

### 6-(3,4-Dimethoxy-phenyl)-2-methyl-3-(6-piperazin-1-yl-pyridin-3-yl)-imidazo[1,2-b]pyridazine (11)

To a solution of 44 mg of 4-{5-[6-(3,4-dimethoxy-phenyl)-2-methyl-imidazo[1,2-b]pyridazin-3-yl]-pyridin-2-yl}-piperazine-1-carboxylic acid tert-butyl ester (Ex. 9) in 1 ml of CH2Cl2, 60.9 μL of TFA is added slowly by a syringe. After 2 h stirring at RT, NaHCO3 is added to neutralize the TFA. The solvent is evaporated, the residue suspended in diethylether and filtered. The title compound is obtained as a yellow solid. ES-MS: (M+1)=431; HPLC: t_{R}=2.8 min.-

### Example 12

### 5-[6-(3,4-Dimethoxy-phenyl)-2-methyl-imidazo[1,2-b]pyridazin-3-yl]-[2,3′]bipyridinyl-6′-carbonitrile (12)

A mixture of 40 mg (0.105 mmol) of 3-(6-chloro-pyridin-3-yl)-6-(3,4-dimethoxy-phenyl)-2-methyl-imidazo[1,2-b]pyridazine (Ex. 10), 30 mg (0.130 mmol) of 2-cyanopyridine-5-boronic acid pinacol ester (Frontier), 3 mg of Pd(dba)₂ (Acros), 68 mg of K3PO4, 4 mg of SPhos, 7.3 mg of Pd(PPh3)2Cl2 (Fluka) in 0.13 ml of 2M Na2CO3-solution, and 1.5 ml of THF are heated in a microwave oven for 30 min at 145° C. (no reaction at 110° C. and 130° C.). After that time, the reaction mixture is poured into CH2Cl2 and washed with water. The organic phase is dried over Na2SO4 and the solvent is evaporated. The residue is purified by combiflash chromatographie on silicagel (solvent system CH2Cl2-EtOAc: 100/0 to 0/100). Further purification is done by chromatographie on preparative HPLC to yield the title compound. ES-MS: (M+1)=449; HPLC: t_{R}=4.779 min.-

### Example 13

### 5-[6-(4-Ethoxy-3-methoxy-phenyl)-2-methyl-imidazo[1,2-b]pyridazin-3-yl]-[2,3]bipyridinyl-6′-carbonitrile (13)

In a 6 ml vial for microwave with crown cap and magnetic stir bar, a mixture of 93 mg (0.236 mmol) of 3-(6-chloro-pyridin-3-yl)-6-(4-ethoxy-3-methoxy-phenyl)-2-methyl-imidazo[1,2-b]pyridazine (preparation see Stage 13.1.), 120 mg (0.522 mmol) of 5-(4,4,5,5-Tetramethyl-[1,3,2]dioxaborolan-2-yl)-pyridine-2-carbonitrile (Frontier), 0.59 ml of 1M aqueous K2CO3-solution, 10 mg of Pd(PPh3)2Cl2 (Fluka) in 3 ml of DMF is heated at 105° C. for 2 h. After that time, the solvent is evaporated under reduced pressure and the residue purified by Combiflash chromatography on silicagel. Solvent system: CH2Cl2-EtOAc 100:0 to 0:100. Fractions containing the product are combined and the solvent evaporated. The residue is suspended in diethylether, filtered and evaporated to dryness to yield the title compound as a yellow solid. ES-MS: (Mil)=463; HPLC: t_{R}=5.137 min.-

### Stage 13.1 (also a compound of the formula I and an Example): 3-(6-Chloro-pyridin-3-yl)-6-(4-ethoxy-3-methoxy-phenyl)-2-methyl-imidazo[1,2-b]pyridazine (13a)

89 mg (0.236 mmol) of 5-[6-(4-ethoxy-3-methoxy-phenyl)-2-methyl-imidazo[1,2-b]pyridazin-3-yl]-pyridin-2-ol (preparation see Stage 13.2.) in 0.216 ml of POCl3 are heated under reflux for 3 h. The mixture is allowed to cool and is poured into ice-water. The water phase is extracted with CH2Cl2, the organic phase washed with water and dried with Na2SO4, filtered and evaporated to dryness. The product is used in the next step without further purification. ES-MS: (M+1)=395; HPLC: t_{R}=4.97 min.-

### Stage 13.2 (also a compound of the formula I and an Example): 5-[6-(4-Ethoxy-3-methoxyphenyl)-2-methyl-imidazo[1,2-b]pyridazin-3-yl]-pyridin-2-ol (13b)

The title compound is prepared in analogy to Example 8 starting from 570 mg (1.37 mmol) of 3-bromo-6-(4-ethoxy-3-methoxy-phenyl)-2-methyl-imidazo[1,2-b]pyridazine (preparation see Stage 13.3.), 364 mg (1.647 mmol) of 2-hydroxypyridine-5-boronic acid, pinacol ester (Boron Molecular), 59 mg of PdCl2(PPh₃)₂ (Fluka), 3.4 ml of 1M aqueous K2CO3-solution and 10 ml of DMF. ES-MS: (M+1)=377; HPLC: t_{R}=3.406 min.-

### Stage 13.3. 3-Bromo-6-(4-ethoxy-3-methoxy-phenyl)-2-methyl-imidazo[1,2-b]pyridazine

388 mg (1.369 mmol) of 6-(4-ethoxy-3-methoxy-phenyl)-2-methyl-imidazo[1,2-b]pyridazine (preparation see Stage 13.4.) and 262 mg (1.4 mmol) of NBS in 40 ml DMF are stirred for 1 h at 0-5° C. and 3 h at RT. The mixture is concentrated at reduced pressure, the residue taken up in CH2Cl2 and washed with water. After drying with Na2SO4, the solvent is evaporated. The residue is suspended in diethylether, stirred at RT and filtered. The title compound is obtained as a beige solid and is used in the next step without further purification. ES-MS: (M+1)=364; HPLC: t_{R}=5.176 min.-

### Stage 13.4. 6-(4-Ethoxy-3-methoxy-phenyl)-2-methyl-imidazo[1,2-b]pyridazine

In a 20 ml vial for microwave with crown cap and magnetic stir bar, a mixture of 600 mg (2.35 mmol) of 2-methoxy-4-(2-methyl-imidazo[1,2-b]pyridazin-6-yl)-phenol (preparation see Stage 13.5.), 0.192 μL (2.38 mmol) ethyliodide, 390 mg (2.822 mmol) of K2CO3 in 15 ml of DMA is stirred under argon at 100° C. for 1 h. The mixture is poured into CH2Cl2 and washed with water. After drying with Na2SO4, the solvent is evaporated and the residue purified by chromatography on silicagel. Solvent system: CH2Cl2-EtOAc 100:0 to 0:100.

Fractions containing the product are combined and the solvent evaporated. The residue is suspended in diethylether, filtered and dried. The title compound is isolated as a colorless solid. ES-MS: (M+1)=284; HPLC: t_{R}=3.895 min.- Stage 13.5: 2-Methoxy-4-(2-methyl-imidazo[1,2-b]pyridazin-6-yl)-phenol.

In a 20 ml vial for microwave with crown cap and magnetic stir bar, a mixture of 250 mg (1.34 mmol) of 6-chloro-2-methyl-imidazo[1,2-b]pyridazine (preparation see Stage 13.6.), 526 mg (1.75 mmol) of 2-methoxy-4-(4,4,5,5-tetramethyl-1,3,2-dioxaborolan-2-yl)phenylacetate Aldrich), 750 mg (5.37 mmol) of K2CO3, 58.7 mg of PdCl2(dppf) (ABCR) in 6 ml dry EtOH and 12 ml of dry toluene is degassed with argon and thereafter heated in the microwave oven at 110° C. for 45 min. The reaction suspension is evaporated to dryness and the residue is dissolved in CH2Cl2. The organic phase is washed with water and brine and dried with Na2SO4. The solvent is evaporated and the crude material purified by chromatography on silicagel to give the title compound. Solvent system: A=CH2Cl2; B=EtOAc-MeOH: 80:20. Chromatography starts with 100% A for 20 min, then 60 min 100% B. ES-MS: (M+1)=256.2; HPLC: t_{R}=2.727 min.-

### Stage 13.6: 6-Chloro-2-methyl-imidazo[1,2-b]pyridazine

A mixture of 5.2 g (38.9 mmol) of 6-chloropyridayzine-3-amine (Maybridge), 6.9 ml (78 mmol) of chloroacetone (Fluka), 13.6 ml (97.3 mmol) of NEt3 in 30 ml of dry EtOH is divided in 3 equal parts, and each part is filled (after degassing with argon) in a 20 ml vials for microwave with crown cap and magnetic stir bar and heated at 150° C. for 30 min. The reaction suspensions are combined and are evaporated to dryness, and the residue is dissolved in CH2Cl2. The organic phase is washed with water and brine, dried with Na2SO4 and evaporated. The raw material is purified by chromatography on silicagel. Solvent system: start with pure CH2Cl2, then CH2Cl2-MeOH 9:1. The title compound is crystallized from diethylether-diisopropylether. ES-MS: (M+1)=168; HPLC: t_{R}=1.696 min.-

### Example 14

### 6-(4-Ethoxy-3-methoxy-phenyl)-2-methyl-3-(6-morpholin-4-yl-pyridin-3-yl)-imidazo[1,2-b]pyridazine (14)

In a 6 ml vial for microwave with crown cap and magnetic stir bar, a mixture of 50 mg (0.138 mmol) of 3-bromo-6-(4-ethoxy-3-methoxy-phenyl)-2-methyl-imidazo[1,2-b]pyridazine (preparation see Stage 13.3.), 58 mg (1.4 mmol) of 2-morpholinepyridine-5-boronic acid pinacol ester (Maybridge), 0.35 ml of 1M aqueous K2CO3-solution, 6 mg of Pd(PPh3)2Cl2 (Fluka) in 1.5 ml of DMF is heated in the microwave oven at 105° C. for 1 h. The mixture is concentrated under reduced pressure, dissolved in CH2Cl2 and washed with water. After drying the organic phase with Na2SO4, the solvent is evaporated and the residue purified by chromatography on C18-silicagel by HPLC. The title compound is obtained as a yellow solid. LC-MS: (M+1)=446; HPLC: t_{R}=3.729 min.-

### Example 15

### 5-[6-(3,4-Dimethoxy-phenyl)-2-methyl-imidazo[1,2-b]pyridazin-3-yl]-3-trifluoromethyl-pyridin-2-ylamine (15)

In a 6 ml vial for microwave with crown cap and magnetic stir bar, 220 mg (ca. 0.277 mmol) of the crude tin compound prepared in Stage 15.1. and 186 mg (0.695 mmol) of 5-bromo-3-trifluoromethyl-pyridin-2-ylamine (for preparation see WO2006/099972-A, p. 87) are dissolved in 3 ml of DMA and degassed with argon. 16 mg of tetrakis-triphenylphosphin palladium are added, and the mixture is heated in the microwave oven at 150° C. for 1 h. The solvent is evaporated, the residue is dissolved in acetonitrile and washed three times with hexane. After evaporating the solvent, the purification is done by chromatography on silicagel. Solvent system: A=CH2Cl2; B=CH2Cl2-MeOH-95:5. Start with 100% A (15 min), then 45 min A:B=1:1, then end with 100% B for 45 min. The title compound is isolated as a yellow solid. LC-MS: (M+1)=430.1; HPLC: t_{R}=4.216 min.-

### Stage 15.1: 6-(3,4-Dimethoxy-phenyl)-2-methyl-3-trimethylstannanyl-imidazo[1,2-b]pyridazine

A solution of 500 mg (1.29 mmol) of 3-bromo-6-(3,4-dimethoxy-phenyl)-2-methyl-imidazo[1,2-b]pyridazine (for preparation see Stage 1.1.) and 0.342 ml (1.62 mmol) of 1,1,1,2,2,2-Hexamethyl-distannane (Fluka) in 10 ml of toluene is degased with argon, then 74.7 mg of tetrakis-triphenylphosphin-palladium are added and the reaction mixture is heated in a sealed tube at 125° C. for 4 h. HPLC and TLC control show that the starting material has been consumed, MS shows the presence of the title compound. The reaction mixture is filtered over hyflo, and the solvent is evaporated. The product is used in the next step without further purification. HPLC indicates a content of ca. 54% of the desired product. ES-MS: (highest peak)=433.9; HPLC: t_{R}=5.272 min.-

### Example 16

### 6-(3,4-Dimethoxy-phenyl)-2-methyl-3-(3-methyl-pyridin-2-yl)-imidazo[1,2-b]pyridazine (16)

The title compound is prepared in analogy to Example 15, starting from 250 mg (ca. 0.315 mmol) raw tin compound from Stage 15.1, 92.5 μL (0.788 mmol) of 2-bromo-3-methyl-pyridine (Aldrich), 18.2 mg of tetrakis-triphenylphosphin-palladium and 3 ml of DMA. Reaction in the microwave oven at 150° C. for 40 min. ES-MS: (M+1)=361.2; HPLC: t_{R}=3.672 min.-

### Example 17

### 6-(3,4-Dimethoxy-phenyl)-2-methyl-3-pyrazin-2-yl-imidazo[1,2-b]pyridazine (17)

The title compound is prepared in analogy to Example 15, starting from 250 mg (ca. 0.315 mmol) raw tin compound from Stage 15.1, 71.8 μL (0.788 mmol) of chloropyrazine (Aldrich), 18.2 mg of tetrakis-triphenylphosphin-palladium and 3 ml of DMA. Reaction in the microwave oven at 150° C. for 60 min. ES-MS: (M+1)=348.2; HPLC: t_{R}=3.837 min.-

### Example 18

### 6-(4′-Methoxy-biphenyl-4-yl)-2-methyl-3-(6-morpholin-4-yl-pyridin-3-yl)-imidazo[1,2-b]pyridazine (18)

In a 6 ml vial for microwave with crown cap and magnetic stir bar, 50 mg (0.127 mmol) of 3-bromo-6-(4′-methoxy-biphenyl-4-yl)-2-methyl-imidazo[1,2-b]pyridazine (preparation see Stage 18.1), 0.32 ml of a 1M K2CO3-solution, 49 mg (0.169 mmol) of 2-morpholinepyridine-5-boronic acid pinacol ester (Maybridge), 5 mg of Pd(PPh3)2Cl2 (Fluka) in 1.5 ml of DMF is degassed with argon and then heated in an oil bath at 105° C. for 2 h 30 min. The solvent is evaporated, the residue taken up in CH2Cl2 and the organic phase washed with water. After drying with Na2SO4 and after evaporating the solvent, the title compound is purified by chromatography on C18-silicagel (preparative HPLC; solvent CH3CN-water)). The compound crystallizes by concentrating the solvent. It is filtered off and is isolated as a yellow solide. ES-MS: (M+1)=478.2; HPLC: t_{R}=4.755 min.-

### Stage 18.1: 3-Bromo-6-(4′-methoxy-biphenyl-4-yl)-2-methyl-imidazo[1,2-b]pyridazine

490 mg (1.554 mmol) of 6-(4′-methoxy-biphenyl-4-yl)-2-methyl-imidazo[1,2-b]pyridazine (preparation see Stage 18.2.) and 297 mg (1.58 mmol) of NBS in 50 ml of DMF are stirred at 0-5° C. for 1 h and additional 2 h at RT. The solvent is evaporated at reduced pressure, the residue is dissolved in CH2Cl2 and the organic phase is washed with water and brine. After drying the solution with Na2SO4, the solvent is evaporated and the residue purified by chromatography on silicagel. Solvent system: CH2Cl2-EtOAc=100:0 to 0:100. The title compound is isolated as a yellow solid. ES-MS: (M+1)=396; HPLC: t_{R}=6.988 min.-

### Stage 18.2: 6-(4′-Methoxy-biphenyl-4-yl)-2-methyl-imidazo[1,2-b]pyridazine

In a 6 ml vial for microwave with crown cap and magnetic stir bar, 300 mg (1.79 mmol) of 6-chloro-2-methyl-imidazo[1,2-b]pyridazine (preparation see Stage 13.6), 500 mg (2.192 mmol) of 4′-methoxybiphenyl-4-ylboronic acid (Combi Blocks), 41 mg of Pd(dba)₂ (Acros), 1.15 g (5.418 mmol) of K3PO4 and 59 mg of SPhos are suspended in 15 ml anhydrous THF and heated in a microwave oven at 110° C. for 30 min. The solvent is evaporated, the residue is dissolved in CH2Cl2 and the organic phase washed with water. After drying with Na2SO4, the solvent is evaporated. The raw material is purified by chromatography on silicagel. Solvent system: CH2Cl2-EtOAc=100:0 to 0:100. The title compound is isolated as a yellow solid. ES-MS: (M+1)=316; HPLC: t_{R}=5.124 min.-

### Example 19

### 5-[6-(4-Methanesulfonyl-phenyl)-2-methyl-imidazo[1,2-b]pyridazin-3-yl]-nicotinonitrile (19)

In a 6 ml vial for microwave with crown cap and magnetic stir bar, a mixture of 50 mg (0.137 mmol) of 3-bromo-6-(4-methanesulfonyl-phenyl)-2-methyl-imidazo[1,2-b]pyridazine (preparation see Stage 19.1), 32 mg (0.139 mmol) of 3-cyanopyridine-5-boronic acid pinacol ester (Frontier), 0.34 ml of a aqueous 1M K2CO3-solution, 6 mg Pd(PPh3)2Cl₂ (Fluka) in 1.5 ml DMF is heated under argon in a oil bath at 105° C. for 4½ h. The reaction mixture is poured into CH2Cl2 and washed with water. After drying with Na2SO4, the solvent is evaporated. The material obtained after chromatography on silicagel is triturated in diethylether to yield the title compound as a yellow solid. ES-MS: (M+1)=390; HPLC: t_{R}=3.732 min.-

### Stage 19.1: 3-Bromo-6-(4-methanesulfonyl-phenyl)-2-methyl-imidazo[1,2-b]pyridazine

603 mg (2.099 mmol) of 6-(4-methanesulfonyl-phenyl)-2-methyl-imidazo[1,2-b]pyridazine (preparation see Stage 19.2), and 401 mg (2.14 mmol) of NBS are stirred in 10 ml of DMF for 1 h at 0-5° C. and 2 more h at RT. The solvent is evaporated, and the residue is dissolved in CH2Cl2. The organic phase is extracted with water and washed with brine. After drying with Na2SO4, the solvent is evaporated. The title compound obtained is used in the next step without further purification. LC-MS: (M+1)=367; HPLC: t_{R}=4.472 min.-

### Stage 19.2: 6-(4-Methanesulfonyl-phenyl)-2-methyl-imidazo[1,2-b]pyridazine

500 mg (2.983 mmol) of 6-chloro-2-methyl-imidazo[1,2-b]pyridazine (preparation see Stage 13.6), 800 mg (3.88 mmol) of (4-methylsulfonylphenylboronic acid (Combi Blocks), 7.5 ml of a aqueous 1M K2CO3-solution, and 117 mg of Pd(PPh3)2Cl2 (Fluka) in 10 ml of DMF are stirred under argon in an oil bath at 105° C. for 5 h. The reaction mixture is poured into CH2Cl2 and extracted with water. After drying with Na2SO4, the solvent is evaporated. The residue is purified by chromatography on silicagel. Solvent system: CH2Cl2-EtOAc: start with 100% CH2Cl2, end with 100 EtOAc. The compound obtained is suspended in diethylether, filtered and dried to yield the desired product as a yellow solid. ES-MS: (M+1)=288; HPLC: t_{R}=2.55 min.-

### Example 20

### 6-(3,4-Dimethoxy-phenyl)-2-methyl-3-(1H-pyrazol-3-yl)-imidazo[1,2-b]pyridazine (20)

In a 6 ml vial for microwave with crown cap and magnetic stir bar, 162 mg (0.442 mmol) of 1-[6-(3,4-dimethoxy-phenyl)-2-methyl-imidazo[1,2-b]pyridazin-3-yl]-3-dimethylamino-propenone (preparation see Stage 20.1) and 63 mg (0.529 mmol) of hydrazine hydrochloride (Fluka) in 1.5 ml dry ethanol are heated at 120° C. for 15 min. The reaction mixture is poured into a solution of saturated NaHCO3 and is extracted with CH2Cl2. The organic layer is dried (Na2SO4), filtered and evaporated to dryness to give the title compound. ES-MS: (M+1)=336; HPLC: t_{R}=3.67 min.-

### Stage 20.1: 1-[6-(3,4-Dimethoxy-phenyl)-2-methyl-imidazo[1,2-b]pyridazin-3-yl]-3-dimethylamino-propenone

In a 6 ml vial for microwave with crown cap and magnetic stir bar, 220 mg (0.707 mmol) of 1-[6-(3,4-dimethoxy-phenyl)-2-methyl-imidazo[1,2-b]pyridazin-3-yl]-ethanone (preparation see Stage 20.2) in 1.8 ml of dimethylformamide dimethylacetale (Fluka) are heated in a microwave oven at 145° C. for 4 h. The mixture is poured into hexane. The title compound precipitates as a beige solid which is filtered off and dried. ES-MS: (M+1)=367; HPLC: t_{R}=3.50 min.-

### Stage 20.2: 1-[6-(3,4-Dimethoxy-phenyl)-2-methyl-imidazo[1,2-b]pyridazin-3-yl]-ethanone

In a 3 ml vial for microwave with crown cap and magnetic stir bar, a mixture of 500 mg (1.51 mmol) of 6-(3,4-dimethoxy-phenyl)-pyridazin-3-ylamine (preparation see Stage 20.3) and 0.19 ml (1.66 mmol) of 3-chloro-2,4-pentanedione (Sigma-Aldrich) in 5 ml dry EtOH is heated in a microwave oven first at 150° C. for 2 h and then, as the reaction is not complete (HPLC-control), for another 90 min at the same temperature. The reaction mixture is evaporated to dryness, the residue taken up in CH2Cl2 and washed with sat. aqueous NaHCO₃-solution. The organic phase is dried (Na2SO4), filtered, and concentrated under reduced pressure. The raw material is purified by chromatography on silicagel to yield the title compound. Solvent system: CH2Cl2-EtOAc: start with 100:0, end with 0:100. ES-MS: (M+1)=312; HPLC: t_{R}=4.402 min.-

### Stage 20.3: 6-(3,4-Dimethoxy-phenyl)-pyridazin-3-ylamine

In a round bottom flask, 500 mg (3.86 mmol) of 3-amino-6-chloropyridazine, 840 mg (2.78 mmol) 3,4-dimethoxyphenylboronic acid, 97.5 mg Pd-catalyst [PdCl₂(PPh₃)₂], and 5.8 ml aqueous K₂CO₃ 1 M are heated under inert conditions in 10 ml DMF at 105° C. for 20 h. After that time, a saturated solution of NaHCO₃ is added and the mixture is extracted with CH₂Cl₂. The organic phase is dried and the solvent evaporated. The residue is purified by chromatography on silica gel. The beige solid is further suspended in methanol, filtered off and dried under high vacuum to yield the title compound. MS-ES.: (M+1)=232; (M−1)=230; HPLC: t_{R}=2.76 min.; LC-MS: t_{R}=1.41 min; (M+1)=232.

### Example 21

### 4-{3-[6-(3,4-Dimethoxy-phenyl)-2-methyl-imidazo[1,2-b]pyridazin-3-yl]-pyrazole-1-sulfonyl}-benzonitrile (21)

To a solution of 40 mg (0.119 mmol) of 6-(3,4-dimethoxy-phenyl)-2-methyl-3-(1H-pyrazol-3-yl)-imidazo[1,2-b]pyridazine (see Example 20) in 1.5 ml of dry pyridine, 47 mg (0.233 mmol) of 4-cyano-benzenesulfonyl chloride (Alfa Aesar) are added. The reactions mixture is stirred at RT for 6 h, then quenched by the addition of saturated aqueous NaHCO3-solution. The product is extracted with CH2Cl2. The organic phase is dried (Na2SO4), filtered and evaporated to dryness. The residue is purified on silicagel by chromatography. Solvent system: CH2Cl2-EtOAc: start with 100:0, end with 0:100. The title compound is isolated as a yellow solid. ES-MS: (M+1)=501; HPLC: t_{R}=5.35 min.-

### Example 22

### 6-(3,4-Dimethoxy-phenyl)-3-[1-(4-methoxy-benzenesulfonyl)-1H-pyrazol-3-yl]-2-methyl-imidazo[1,2-b]pyridazine (22)

The title compound is prepared in analogy to Example 21. The title compound is isolated as a yellow solid. ES-MS: (M+1)=506; HPLC: t_{R}=5.458 min.-

### Example 23

### 3-{3-[6-(3,4-Dimethoxy-phenyl)-2-methyl-imidazo[1,2-b]pyridazin-3-yl]-pyrazole-1-sulfonyl}-benzonitrile (23)

The title compound is prepared in analogy to Example 22. The product is isolated as a yellow solid. ES-MS: (M+1)=501; HPLC: t_{R}=5.314 min.-

### Example 24

### 5-{6-[4-(2-Amino-ethoxy)-3-methoxy-phenyl]-2-methyl-imidazo[1,2-b]pyridazin-3-yl}-3-trifluoromethyl-pyridin-2-ylamine (24)

To a solution of 76 mg (0.129 mmol) of (2-{4-[3-(6-amino-5-trifluoromethyl-pyridin-3-yl)-2-methyl-imidazo[1,2-b]pyridazin-6-yl]-2-methoxy-phenoxy}-ethyl)-carbamic acid tert-butylester (preparation see Stage 24.1) in 2 ml of CH2Cl2 are added 0.25 ml of mixture of trifluoro acetic acid-water 9:1. The reaction mixture is stirred at RT for 3 h (HPLC and MS-control). After this period, the reaction mixture is cooled to 0-5° C. (ice water), and 1 ml of 6M NH3 in EtOH is added and stirred for 10 min. Ca. 1 g of silicagel is added, the solvents are evaporated and the compound absorbed on the silicagel matrix is purified by chromatography. Solvent system: A: CH2Cl2; B: CH2Cl2-MeOH—NH4OH 32%=90:10:1. Start with 15 min with A, then 20 min B. The title compound is isolated as a yellow solid. ES-MS: (M+1)=459.1; HPLC: t_{R}=2.956 min.-

### Stage 24.1: (also a compound of the formula I according to the invention, that is, an Example) (2-{4-[3-(6-Amino-5-trifluoromethyl-pyridin-3-yl)-2-methyl-imidazo[1,2-b]pyridazin-6-yl]-2-methoxy-phenoxy}-ethyl)-carbamic acid tert-butyl ester (24a)

In a 6 ml vial for microwave with crown cap and magnetic stir bar, 152 mg (0.302 mmol) of {2-[4-(3-bromo-2-methyl-imidazo[1,2-b]pyridazin-6-yl)-2-methoxy-phenoxy]-ethyl}-carbamic acid tert-butyl ester (preparation see Stage 24.2.), 174 mg (0.605 mmol) of 5-(4,4,5,5-tetramethyl-[1,3,2]dioxaborolan-2-yl)-3-trifluoromethyl-pyridin-2-ylamine (preparation see Stage 24.3.), and 0.76 ml of 1M aqueous K2CO3-solution in 4 ml of DMA are degassed with argon. Then 10.8 mg of Pd(PPh3)2Cl2 (Fluka) are added and the mixture is heated in the microwave oven at 150° C. for 30 min. The reaction mixture is evaporated to dryness, the residue taken up in CH2Cl2. The organic phase is washed with water and brine, dried with Na2SO4 and evaporated. Purification is done by chromatography on silicagel to yield the title compound. Solvent system: A: CH2Cl2; B: CH2Cl2-MeOH=98:2. Start with 20 min with A, then 30 min B. ES-MS: (M+1)=559; HPLC: t_{R}=4.92 min.-

### Stage 24.2: {2-[4-(3-Bromo-2-methyl-imidazo[1,2-b]pyridazin-6-yl)-2-methoxy-phenoxy]-ethyl}-carbamic acid tert-butylester

A solution of 732 mg (1.75 mmol) of {2-[2-methoxy-4-(2-methyl-imidazo[1,2-b]pyridazin-6-yl)-phenoxy]-ethyl}-carbamic acid tert-butyl ester (preparation see Stage 24.5.) in 7.3 ml of DMF is cooled down to 0-5° C. Argon is passed through the reaction mixture. NBS is added in one portion and the mixture is stirred at the same temperature for 2 h. The reaction mixture is poured into EtOAc and the organic phase is washed with water and brine. The organic phase is dried and evaporated to dryness to give the title compound. No further purification is done. ES-MS: (M+1)=478.9; HPLC: t_{R}=5.486 min.-

### Stage 24.3: 5-(4,4,5,5-Tetramethyl-[1,3,2]dioxaborolan-2-yl)-3-trifluoromethyl-pyridin-2-yl-amine

8.04 g (31.7 mmol) of 5-bromo-3-trifluoromethyl-pyridin-2-ylamine (preparation see Stage 24.4.), 10.5 g (41.2 mmol) of 4,4,5,5,4′,4′,5′,5′-octamethyl-[2,2′]bi[[1,3,2]dioxaborolanyl] (Aldrich), 9.62 g (95.1 mmol) of KOAc in 100 ml dioxane are degassed with argon for 15 min. Then 776 mg (0.951 mmol) of bis(diphenylphosphino)ferrocene dichloropalladium(II)dichloromethane (ABCR) are added and the mixture is degassed for 15 more minutes. The reaction mixture is heated at 115° C. for 8 h. After that time, the reaction mixture is filtered and the solvent evaporated. The residue is purified by simple filtration on silicagel (solvent system: t-butyl-methyl ether-EtOAc-NEt3=50:50:0.1) to yield the title compound as almost colorless solid. ES-MS: (M+1)=289; Tlc: Rf=0.77 in t-buthyl-methyl ether-EtOAc 1:1.-

### Stage 24.4: 5-Bromo-3-trifluoromethyl-pyridin-2-ylamine

To a solution of 5.37 g (32.8 mmol) of 3-trifluoromethyl-pyridin-2-ylamine (Fluorochem) in 100 ml of dry CH3CN, 6.45 g of NBS are added in 4 equal portions over a period of 1 h at 0-5° C. under argon. The cooling bath is removed and stirring is continued for 3 h. The solvent is evaporated under vacuum, the residue is dissolved in EtOAc and washed with water and brine. The organic phase is dried over Na2SO4 and evaporated. The title compound is a reddish-yellow oil which is used after drying in the dark for 5 h at RT and under high vacuum in the next step without further purification. ES-MS (M+1):=239; 241; HPLC: t_{R}=5.501 min.-

### Stage 24.5: {2-[2-Methoxy-4-(2-methyl-imidazo[1,2-b]pyridazin-6-yl)-phenoxy]-ethyl}-carbamic acid tert-butyl ester

500 mg (1.86 mmol) of 2-methoxy-4-(2-methyl-imidazo[1,2-b]pyridazin-6-yl)-phenol (preparation see Stage 13.5), 860 mg (3.72 mmol) of 2-(Boc-amino)ethyl bromide (Fluka), and 1.12 g (4.65 mmol) of K2CO3 in 10 ml of dry DMA are heated in a sealed vial in a oil bath at 100° C. After 2.5 h starting material can still be detected. Additional Boc-amino reagent (344 mg; 0.8 equivalents) is added. The reaction is finished after 8 h. The solvent is evaporated to dryness, the residue is taken up in EtOAc and is washed with water and brine. The organic phase is dried over Na2SO4 with the addition of charcoal, the solution is filtered over hyflo, and the solvent is evaporated. Purification is done by chromatography on silicagel. Solvent system: A=CH2Cl2; B=CH2Cl2-MeOH: 98/2. Start with A for 15 min, then B for a total of 40 min. ES-MS (M+1): =399.2; HPLC: t_{R}=4.47 min.-

### Example 25

### 5-{6-[4-(3-Amino-propoxy)-3-methoxy-phenyl]-2-methyl-imidazo[1,2-b]pyridazin-3-yl}-3-trifluoromethyl-pyridin-2-ylamine (25)

The title compound is prepared in analogy to Example 24 starting from 98 mg (0.163 mmol) of (3-{4-[3-(6-amino-5-trifluoromethyl-pyridin-3-yl)-2-methyl-imidazo[1,2-b]pyridazin-6-yl]-2-methoxy-phenoxy}-propyl)-carbamic acid tert-butyl ester (preparation see Stage 25.1). ES-MS (M+1):=473.1; HPLC: t_{R}=3.097 min.-

### Stage 25.1: (also a compound of the formula I according to the invention, that is, an Example): (3-{4-[3-(6-Amino-5-trifluoromethyl-pyridin-3-yl)-2-methyl-imidazo[1,2-b]pyridazin-6-yl]-2-methoxy-phenoxy}-propyl)-carbamic acid tert-butyl ester (25a)

The title compound is prepared in analogy to the compound prepared in Stage 24.2. starting from 150 mg (0.29 mmol) of {3-[4-(3-bromo-2-methyl-imidazo[1,2-b]pyridazin-6-yl)-2-methoxy-phenoxy]-propyl}-carbamic acid tert-butyl ester (preparation see Stage 25.2.) and 167 mg (0.58 mmol) of the boronic acid prepared in Stage 24.3. ES-MS (M+1):=573.1; HPLC: t_{R}=5.128 min.-

### Stage 25.2: {3-[4-(3-Bromo-2-methyl-imidazo[1,2-b]pyridazin-6-yl)-2-methoxy-phenoxy]-propyl}-carbamic acid tert-butyl ester

The title compound is prepared in analogy to the compound prepared in Stage 24.2. starting from 660 mg (1.52 mmol) of {3-[2-methoxy-4-(2-methyl-imidazo[1,2-b]pyridazin-6-yl)-phenoxy]-propyl}-carbamic acid tert-butyl ester (preparation see Stage 25.3.) and 290 mg (1.55 mmol) of NBS. ES-MS:=491; 493; HPLC: t_{R}=5.788 min.-

### Stage 25.3: {3-[2-Methoxy-4-(2-methyl-imidazo[1,2-b]pyridazin-6-yl)-phenoxy]-propyl}-carbamic acid tert-butyl ester

The title compound is prepared in analogy to the compound prepared in Stage 24.5. starting from 500 mg (1.86 mmol) of 2-methoxy-4-(2-methyl-imidazo[1,2-b]pyridazin-6-yl)phenol (preparation see Stage 13.5.) and 3-(Boc-amino)propyl bromide (Fluka). ES-MS (M+1):=413.2; HPLC: t_{R}=4.712 min.-

Further Examples The following compounds are manufactured according to methods described herein or by reactions as described in the following reaction schemes:

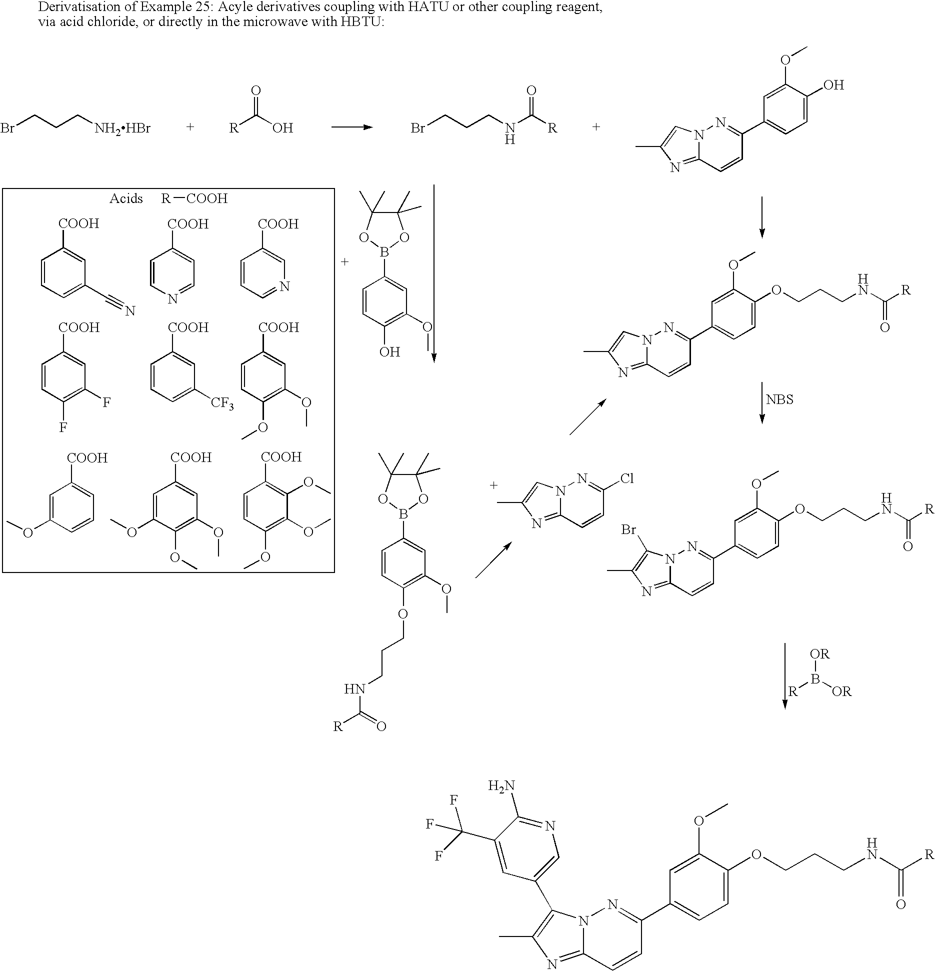

Analogously, the ethyl derivatives (analogues of Example 24) can be prepared

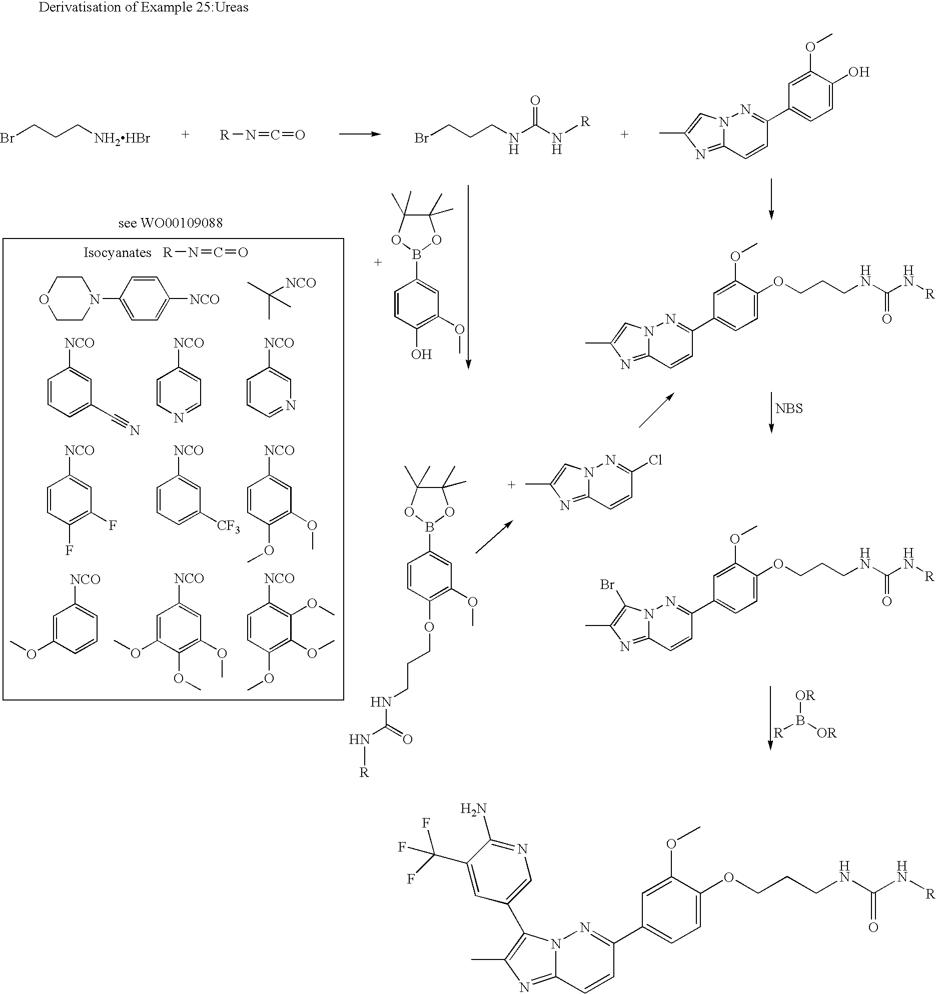

Analogously, the ethyl derivatives (analogues of Example 24) can be prepared.

The compounds which are of the formula (26A) below are given in the following table together with some data (the asterisk (*) marks the end of the bond with which the corresponding moiety Rvar is bound to the oxygen of the rest of the molecule):

(26A)

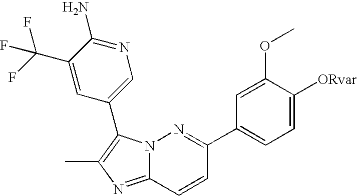

| Example | Rvar | ESI-MS+ | HPLC |
|---|---|---|---|
| 26 | 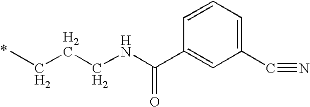 | | |
| 27 | 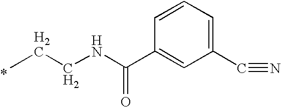 | 588 | 4.484 |
| 28 | 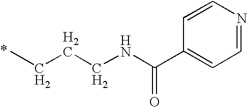 | | |
| 29 | 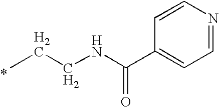 | 564 | 3.337 |
| 30 | 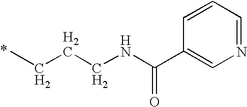 | | |
| 31 | 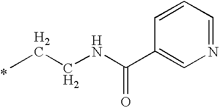 | 564 | 3.369 |
| 32 | 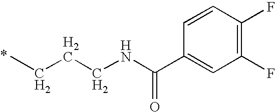 | 599 | 4.844 |
| 33 | 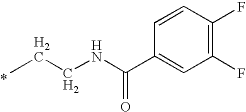 | | |
| 34 | 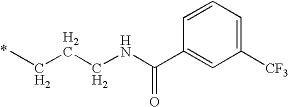 | | |
| 35 | 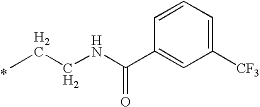 | | |
| 36 | 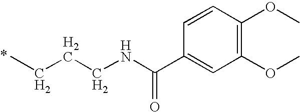 | | |
| 37 | 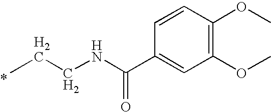 | 623 | 4.31 |
| 38 | 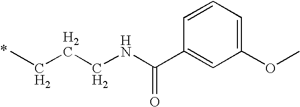 | | |
| 39 | 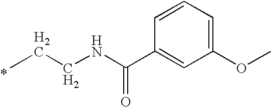 | 593 | 4.585 |
| 40 | 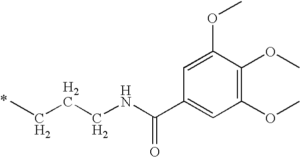 | | |
| 41 | 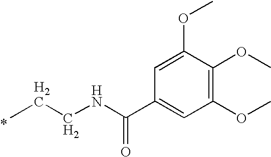 | 653 | 4.461 |
| 42 | 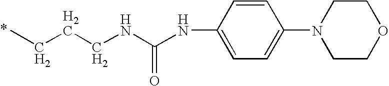 | | |
| 43 | 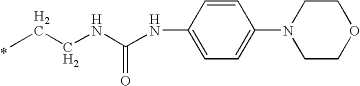 | | |
| 44 | 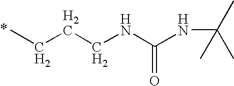 | | |
| 45 | 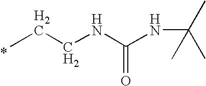 | | |
| 46 | 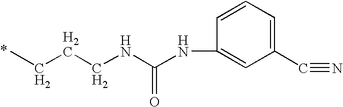 | | |
| 47 | 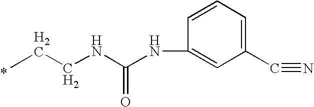 | 603 | 4.616 |
| 48 | 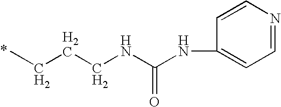 | | |
| 49 | 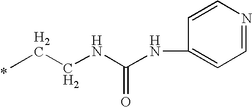 | | |
| 50 | 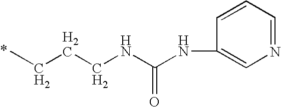 | | |
| 51 | 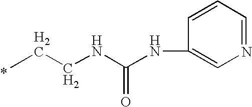 | 579 | 3.364 |
| 52 | 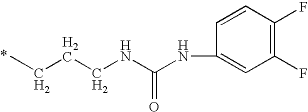 | | |
| 53 | 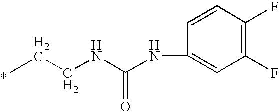 | | |
| 54 | 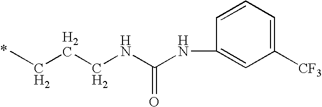 | | |
| 55 | 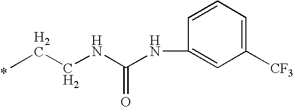 | 631 | 5.141 |
| 56 | 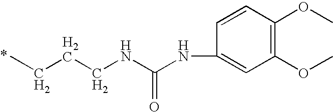 | | |
| 57 | 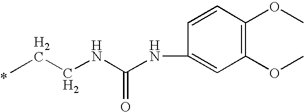 | | |
| 58 | 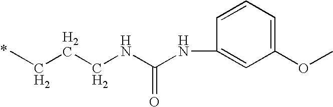 | 622 | 4.694 |
| 59 | 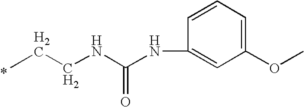 | 608 | 4.624 |
| 60 | 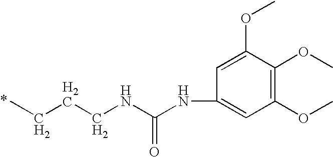 | | |
| 61 | 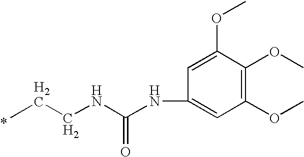 | 668 | 4.326 |
| 62 | 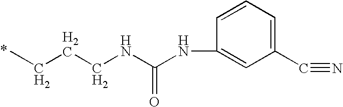 | | |
| 63 | 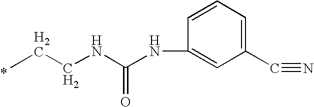 | | |

The following Examples are also compounds of the formula (26A) given above and are prepared by or in analogy to methods described herein, or as mentioned specifically:

| Example | Rvar | | |
|---|---|---|---|
| 64 | 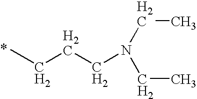 | | |
| 65 | 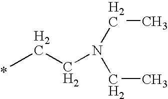 | | |
| 66 | 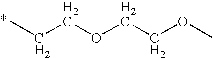 | | |
| 67 | 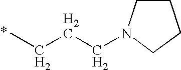 | | |
| 68 | 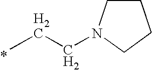 | | |
| 69 | 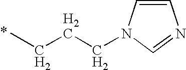 | | |
| 70 | 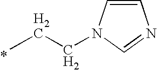 | | |
| 71 | 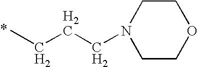 | | |
| 72 | 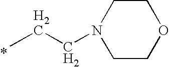 | | |
| 73 | 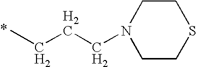 | | |
| 74 | 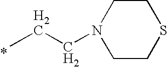 | | |
| 75 | 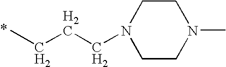 | | |
| 76 | 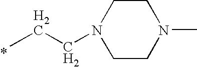 | | |
| 77 | 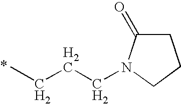 | 541 | 4.074 |
| 78 | 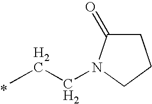 | | |

These compounds can be prepared in analogy to a compound of the formula 25 or 24, but with RVar instead of the 2-aminoethyl or 3-aminopropyl group. The corresponding precursors (or also the compounds themselves) can be prepared from the starting materials wherein OH is present by reaction with a compound Rvar-Z wherein Z is halo, especially chloro or bromo, or if Z is OH by reaction under Mitsunobu conditions for arylether synthesis.

A starting material for Example 69 (and analogously for Example 70) can be prepared as follows:

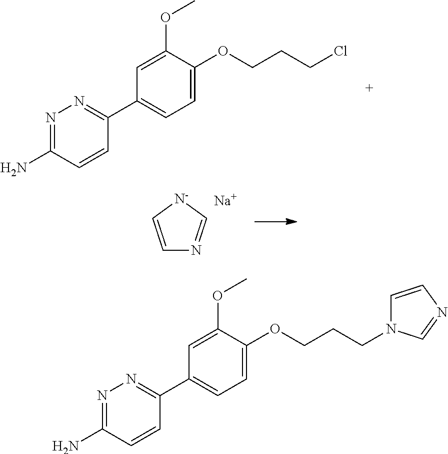

The following Examples are also compounds of the formula (26) given above and are prepared by or in analogy to methods described herein, or as mentioned specifically:

| Example | Rvar | | |
|---|---|---|---|
| 79 | 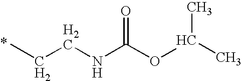 | 545 | 4.603 |
| 80 | 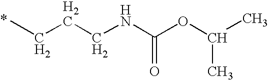 | 559.1 | 4.8 |
| 81 | 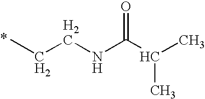 | 529 | 4.07 |
| 82 | 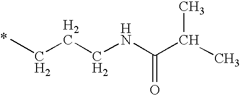 | 543.1 | 4.246 |

| EX- | | | | | |
|---|---|---|---|---|---|
| AM- | | | | | |
| PLE | | | | | |
| NUM- | | | method of | ESI- | |
| BER | structure | compound name | preparation | MS+ | HPLC |
| 83 | 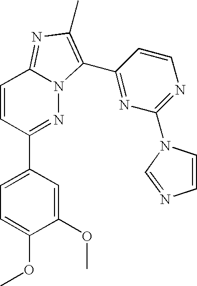 | 6-(3,4-Dimethoxy- phenyl)-3-(2-imidazol-1- yl-pyrimidin-4-yl)-2- methyl-imidazo[1,2- b]pyridazine | Method A | 414.1 | 3.85 |
| 84 | 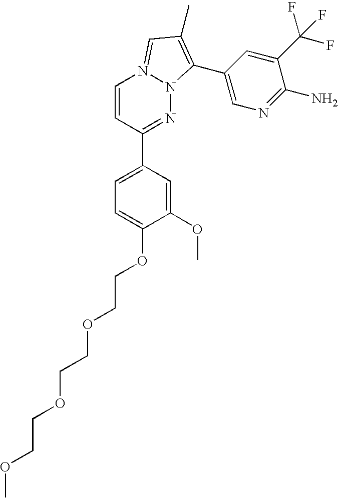 | 5-[6-(3-Methoxy-4-{2-[2- (2-methoxy-ethoxy)- ethoxy]-ethoxy}-phenyl)- 2-methyl-imidazo[1,2- b]pyridazin-3-yl]-3- trifluoromethyl-pyridin-2- ylamine | same as example 24 (stage 24.1-24.5) using 1-chloro-2-[2-(2- methoxy-ethoxy)- ethoxy]-ethane instead | 562.1 | 4.219 |
| 85 | 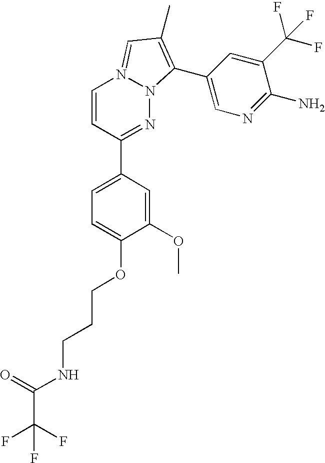 | N-(3-{4-[3-(6-Amino-5- trifluoromethyl-pyridin-3- yl)-2-methyl-imidazo[1,2- b]pyridazin-6-yl]-2- methoxy-phenoxy}- propyl)-2,2,2-trifluoro- acetamide | side product of example 25 | 568.6 | 4.725 |
| 86 | 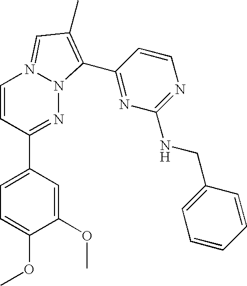 | Benzyl-{4-[6-(3,4- dimethoxy-phenyl)-2- methyl-imidazo[1,2- b]pyridazin-3-yl]- pyrimidin-2-yl}-amine | Method A using benzylamine instead | 453.2 | 5.096 |
| 87 | 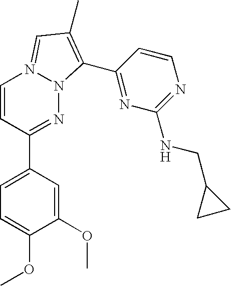 | Cyclopropylmethyl-{4-[6- (3,4-dimethoxy-phenyl)-2- methyl-imidazo[1,2- b]pyridazin-3-yl]- pyrimidin-2-yl}-amine | Method A using cyclopropanemethyl amine instead | 417.2 | 4.76 |
| 88 | 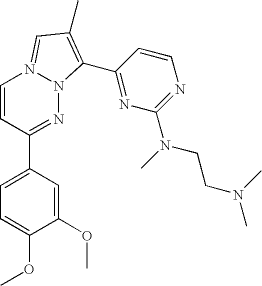 | N-{4-[6-(3,4-Dimethoxy- phenyl)-2-methyl- imidazo[1,2-b]pyridazin-3- yl]-pyrimidin-2-yl}-N,N′,N′- trimethyl-ethane-1,2- diamine | Method A using N,N,N- trimethylendiamine instead | 448.2 | 3.522 |
| 89 | 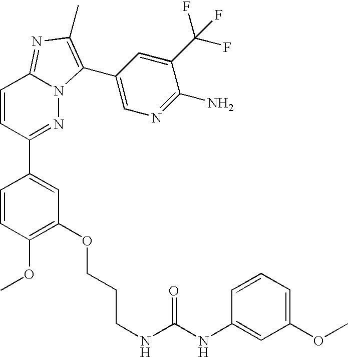 | 1-(3-{5-[3-(6-Amino-5- trifluoromethyl-pyridin-3- yl)-2-methyl-imidazo[1,2- b]pyridazin-6-yl]-2- methoxy-phenoxy}- propyl)-3-(3-methoxy- phenyl)-urea | see method B | 622 | 4.691 |
| 90 | 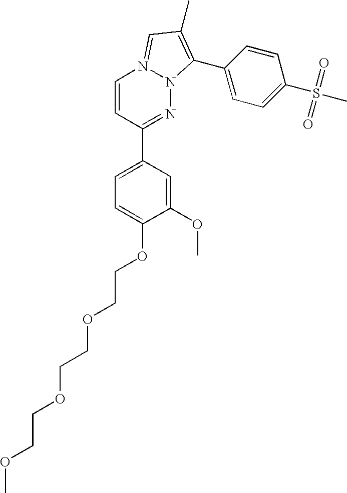 | 3-(4-Methanesulfonyl- phenyl)-6-(3-methoxy-4- {2-[2-(2-methoxy-ethoxy)- ethoxy]-ethoxy}-phenyl)- 2-methyl-imidazo[1,2- b]pyridazine | same as example 84 using 4- methanesulfonyl- boronic acid instead | 556.1 | 4.141 |
| 91 | 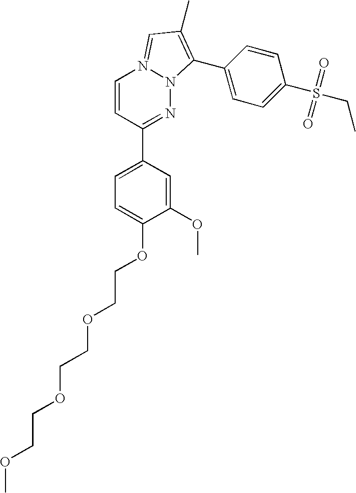 | 3-(4-Ethanesulfonyl- phenyl)-6-(3-methoxy-4- {2-[2-(2-methoxy-ethoxy)- ethoxy]-ethoxy}-phenyl)- 2-methyl-imidazo[1,2- b]pyridazine | same as example 84 using 4- ethanesulfonyl- boronic acid instead | 570 | 4.354 |
| 92 | 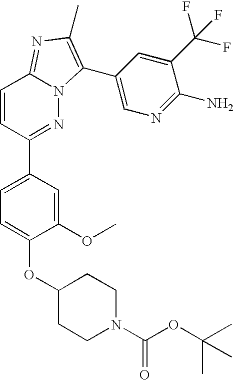 | 4-{4-[3-(6-Amino-5- trifluoromethyl-pyridin-3- yl)-2-methyl-imidazo[1,2- b]pyridazin-6-yl]-2- methoxy-phenoxy}- piperidine-1-carboxylic acid tert-butyl ester | same as example 24 (stage 24.1-24.5) using 4- methanesulfonyloxy- piperidine-1- carboxylic acid tert-butyl ester instead | 599 | 5.585 |
| 93 | 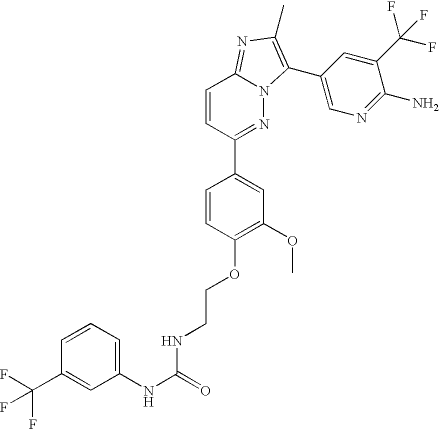 | 1-(2-{4-[3-(6-Amino-5- trifluoromethyl-pyridin-3- yl)-2-methyl-imidazo[1,2- b]pyridazin-6-yl]-2- methoxy-phenoxy}-ethyl)- 3-(3-trifluoromethyl- phenyl)-urea | same as example 89 using 1-isocyanato-3- trifluoromethyl- benzene instead | 646 | 5.285 |
| 94 | 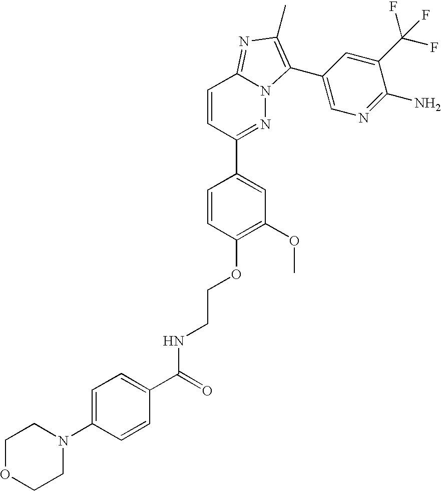 | N-(2-{4-[3-(6-Amino-5- trifluoromethyl-pyridin-3- yl)-2-methyl-imidazo[1,2- b]pyridazin-6-yl]-2- methoxy-phenoxy}-ethyl)- 4-morpholin-4-yl- benzamide | see method C | 648 | 4.327 |
| 95 | 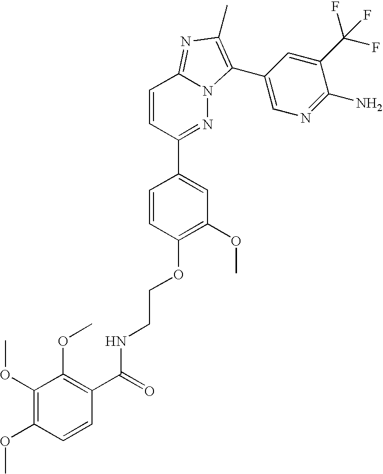 | N-(2-{4-[3-(6-Amino-5- trifluoromethyl-pyridin-3- yl)-2-methyl-imidazo[1,2- b]pyridazin-6-yl]-2- methoxy-phenoxy}-ethyl)- 2,3,4-trimethoxy- benzamide | same as example 94 using 2,3,4- trimethoxy- benzoic acid | 653 | 4.751 |
| 96 | 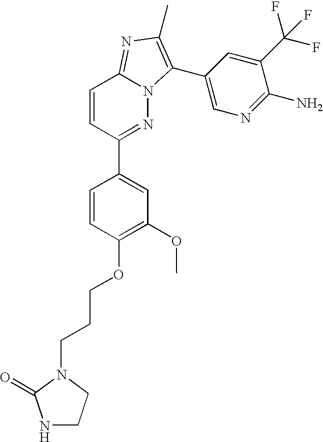 | 1-(3-{4-[3-(6-Amino-5- trifluoromethyl-pyridin-3- yl)-2-methyl-imidazo[1,2- b]pyridazin-6-yl]-2- methoxy-phenoxy}- propyl)-imidazolidin-2-one | see method D | 542 | 3.865 |
| 97 | 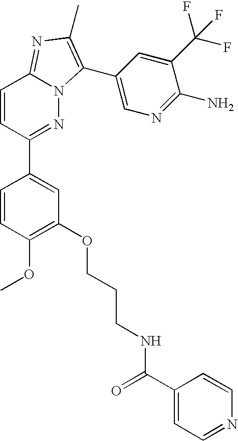 | N-(3-{5-[3-(6-Amino-5- trifluoromethyl-pyridin-3- yl)-2-methyl-imidazo[1,2- b]pyridazin-6-yl]-2- methoxy-phenoxy}- propyl)-isonicotinamide | same as example 94 using 2- methoxy-5-(2- methyl-imidazo[1,2- b]pyridazin-6-yl)- phenol and isonicotinic acid instead | 578 | 3.494 |
| 98 | 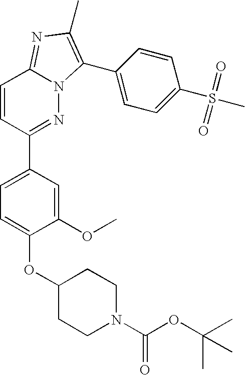 | 4-{4-[3-(4- Methanesulfonyl-phenyl)- 2-methyl-imidazo[1,2- b]pyridazin-6-yl]-2- methoxy-phenoxy}- piperidine-1-carboxylic acid tert-butyl ester | same as example 92 using 4- methanesulfonyl- boronic acid instead | 593.2 | 5.504 |
| 99 | 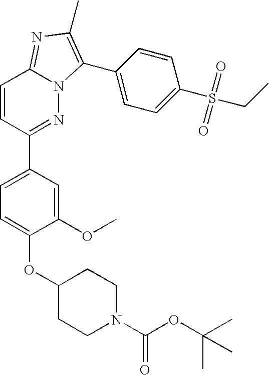 | 4-{4-[3-(4-Ethanesulfonyl- phenyl)-2-methyl- imidazo[1,2-b]pyridazin-6- yl]-2-methoxy-phenoxy}- piperidine-1-carboxylic acid tert-butyl ester | same as example 92 using 4- ethanesulfonyl- boronic acid instead | 607 | 5.693 |
| 100 | 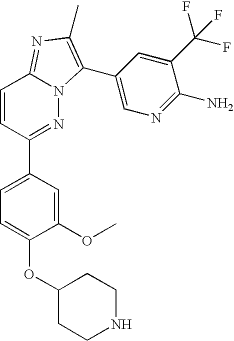 | 5-{6-[3-Methoxy-4- (piperidin-4-yloxy)- phenyl]-2-methyl- imidazo[1,2-b]pyridazin-3- yl}-3-trifluoromethyl- pyridin-2-ylamine | same as example 11 starting from 4-{4-[3-(6- Amino-5- trifluoromethyl- pyridin-3-yl)-2- methyl- imidazo[1,2-b] pyridazin- 6-yl]-2-methoxy- phenoxy}- piperidine-1- carboxylic acid tert-butyl ester (example 92) | 498 | 3.18 |
| 101 | 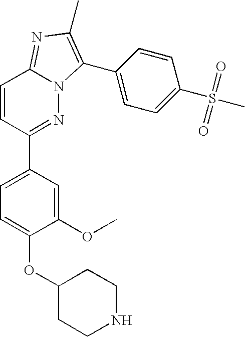 | 3-(4-Methanesulfonyl- phenyl)-6-[3-methoxy-4- (piperidin-4-yloxy)- phenyl]-2-methyl- imidazo[1,2-b]pyridazine | same as example 11 starting from 4-{4-[3- (4- Methanesulfonyl- phenyl)-2-methyl imidazo[1,2-b] pyridazin- 6-yl]-2-methoxy- phenoxy}- piperidine-1- carboxylic acid tert-butyl ester (example 98) | 493.1 | 3.072 |
| 102 | 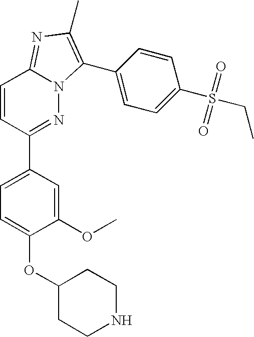 | 3-(4-Ethanesulfonyl- phenyl)-6-[3-methoxy-4- (piperidin-4-yloxy)- phenyl]-2-methyl- imidazo[1,2-b]pyridazine | same as example 11 starting from 4-{4- [3-(4- Ethanesulfonyl- phenyl)- 2-methyl-imidazo[1,2- b]pyridazin-6-yl]-2- methoxy-phenoxy}- piperidine-1- carboxylic acid tert-butyl ester (example 99) | 507 | 3.295 |
| 103 | 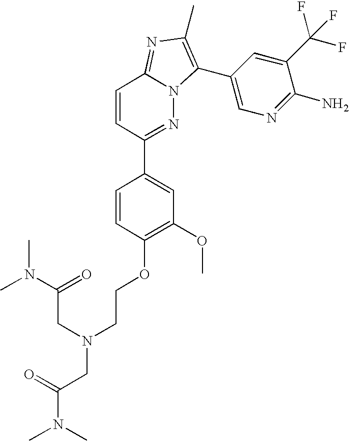 | 2-[(2-{4-[3-(6-Amino-5- trifluoromethyl-pyridin-3- yl)-2-methyl-imidazol[1,2- b]pyridazin-6-yl]-2- methoxy-phenoxy}-ethyl)- dimethylcarbamoylmethyl- amino]-N,N-dimethyl- acetamide | same as example 96 using 5-{6-[4-(3- Amino- propoxy)-3-methoxy- phenyl]-2-methyl- imidazo[1,2-b] pyridazin- 3-yl}-3- trifluoromethyl- pyridin-2-ylamine (example 25) and N,N- dimethyl- chloroacetamide instead | 629 | 3.302 |
| 104 | 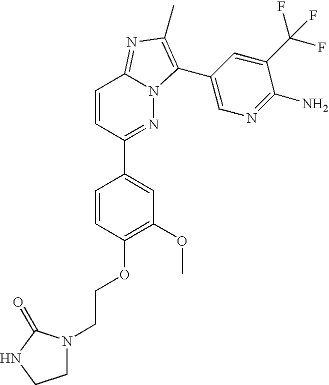 | 1-(2-{4-[3-(6-Amino-5- trifluoromethyl-pyridin-3- yl)-2-methyl-imidazo[1,2- b]pyridazin-6-yl]-2- methoxy-phenoxy}-ethyl)- imidazolidin-2-one | same as example 24 (stage 24.1-24.5) using methanesulfonic acid 2- (2-oxo- imidazolidin-1-yl)- ethyl ester instead | 528 | 3.713 |
| 105 | 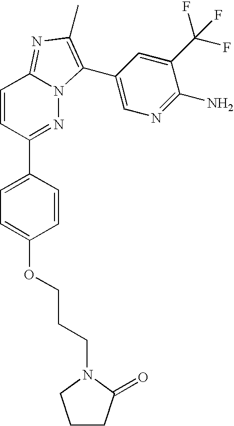 | 1-(3-{4-[3-(6-Amino-5- trifluoromethyl-pyridin-3- yl)-2-methyl-imidazo[1,2- b]pyridazin-6-yl]- phenoxy}-propyl)- pyrrolidin-2-one | same as example 24 (stage 24.1-24.5) using 4-hydroxyphenyl- boronic acid and methanesulfonic acid 3- (2-oxo-pyrrolidin- 1-yl)-propyl ester instead | 511.1 | 4.206 |
| 106 | 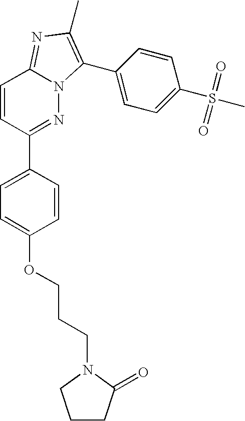 | 1-(3-{4-[3-(4- Methanesulfonyl-phenyl)- 2-methyl-imidazo[1,2- b]pyridazin-6-yl]- phenoxy}-propyl)- pyrrolidin-2-one | same as example 105 using 4- methanesulfonyl- boronic acid instead | 505.1 | 4.104 |
| 107 | 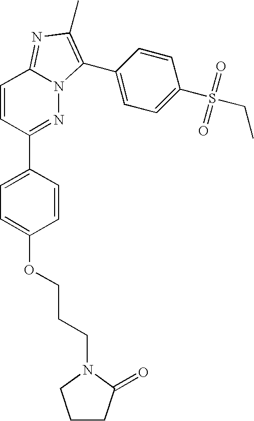 | 1-(3-{4-[3-(4- Ethanesulfonyl-phenyl)-2- methyl-imidazo[1,2- b]pyridazin-6-yl]- phenoxy}-propyl)- pyrrolidin-2-one | same as example 105 using 4-ethanesulfonyl- boronic acid instead | 519.1 | 4.307 |
| 108 | 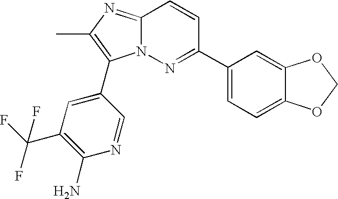 | 5-(6-Benzo[1,3]dioxol-5- yl-2-methyl-imidazo[1,2- b]pyridazin-3-yl)-3- trifluoromethyl-pyridin-2- ylamine | same as example 19 using benzo[1,3] dioxole- 5-carboxylic acid and 5- (4,4,5,5-tetramethyl- [1,3,2]dioxaborolan-2- yl)-3-trifluoromethyl- pyridin-2-ylamine (example 24; 24.3) instead | 414 | 4.358 |
| 109 | 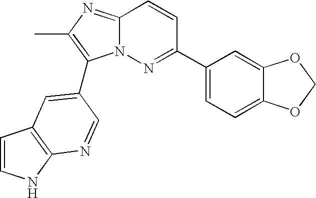 | 6-Benzo[1,3]dioxol-5-yl-2- methyl-3-(1H-pyrrolo[2,3- b]pyridin-5-yl)- imidazo[1,2-b]pyridazine | same as example 108 using 5-(4,4,5,5- tetramethyl- [1,3,2]dioxaborolan-2- yl)-1H-pyrrolo[2,3- b]pyridine instead | 370.1 | 4.117 |
| 110 | 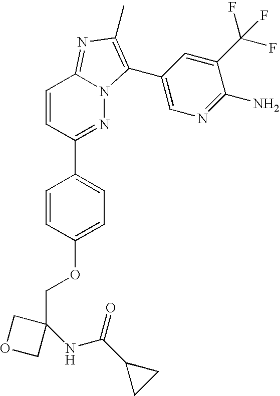 | Cyclopropanecarboxylic acid (3-{4-[3-(6-amino-5- trifluoromethyl-pyridin-3- yl)-2-methyl-imidazo[1,2- b]pyridazin-6-yl]- phenoxymethyl}-oxetan-3- yl)-amide | see text | | |
| 111 | 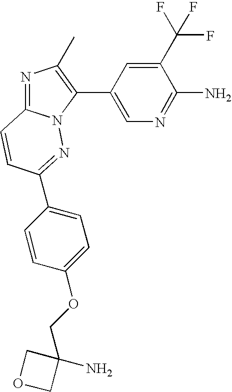 | 5-{6-[4-(3-Amino-oxetan- 3-ylmethoxy)-phenyl]-2- methyl-imidazo[1,2- b]pyridazin-3-yl}-3- trifluoromethyl-pyridin-2- ylamine | see text | | |
| 112 | 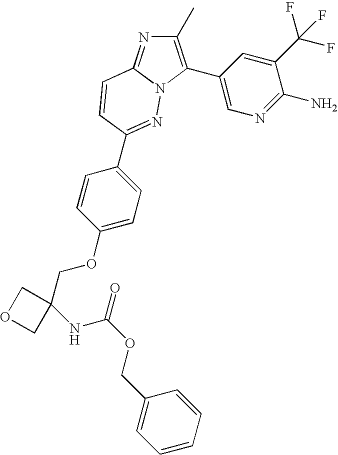 | (3-{4-[3-(6-Amino-5- trifluoromethyl-pyridin-3- yl)-2-methyl-imidazo[1,2- b]pyridazin-6-yl]- phenoxymethyl}-oxetan-3- yl)-carbamic acid benzyl ester | see text | | |
| 113 | 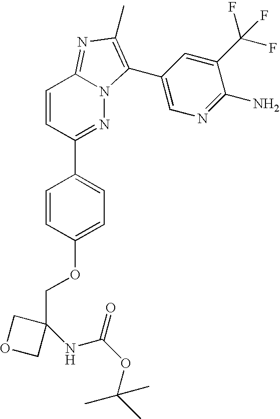 | (3-{4-[3-(6-Amino-5- trifluoromethyl-pyridin-3- yl)-2-methyl-imidazo[1,2- b]pyridazin-6-yl]- phenoxymethyl}-oxetan-3- yl)-carbamic acid tert- butyl ester | see text | | |
| 114 | 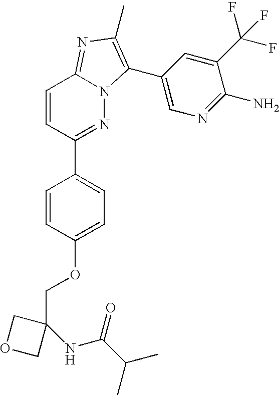 | N-(3-{4-[3-(6-Amino-5- trifluoromethyl-pyridin-3- yl)-2-methyl-innidazo[1,2- b]pyridazin-6-yl]- phenoxymethyl}-oxetan-3- yl)-isobutyramide | see text | | |
| 115 | 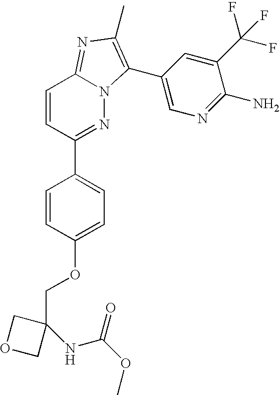 | (3-{4-[3-(6-Amino-5- trifluoromethyl-pyridin-3- yl)-2-methyl-imidazo[1,2- b]pyridazin-6-yl]- phenoxymethyl}-oxetan-3- yl)-carbamic acid methyl ester | see text | | |
| 116 | 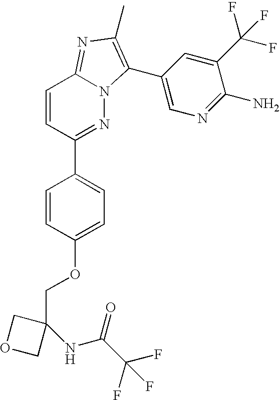 | N-(3-{4-[3-(6-Amino-5- trifluoromethyl-pyridin-3- yl)-2-methyl-imidazo[1,2- b]pyridazin-6-yl]- phenoxymethyl}-oxetan-3- yl)-2,2,2-trifluoro- acetamide | see text | | |

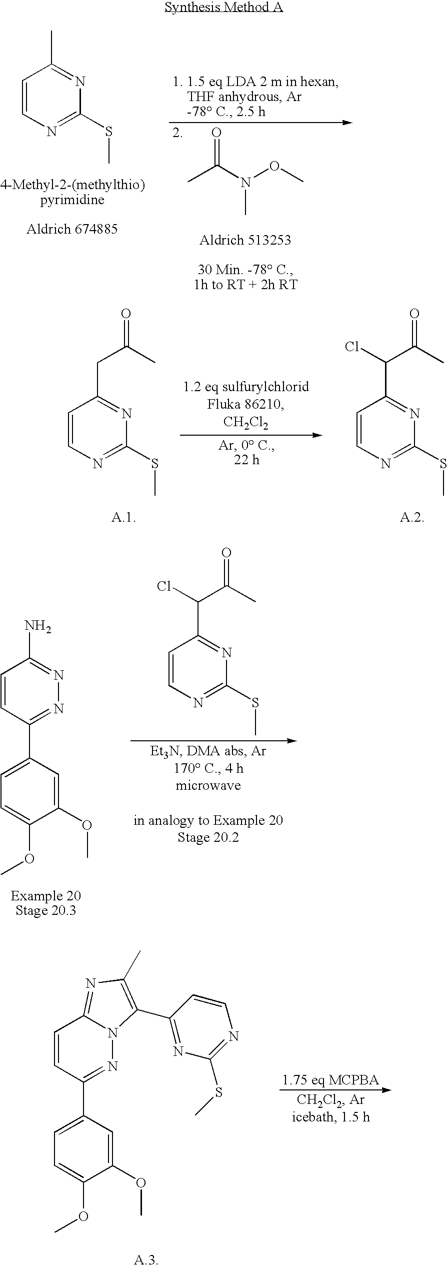

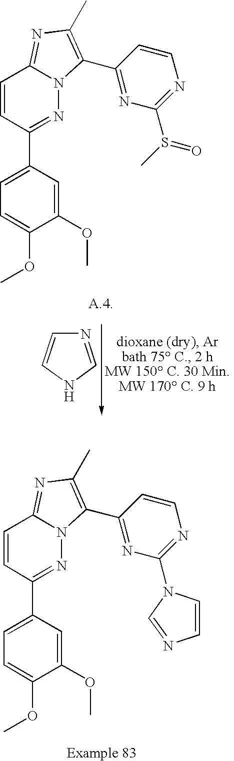

### Example 83

### 6-(3,4-Dimethoxy-phenyl)-3-(2-imidazol-1-yl-pyrimidin-4-yl)-2-methyl-imidazo[1,2-b]pyridazine

To a dry vial (argon flushed) containing 6-(3,4-Dimethoxy-phenyl)-3-(2-methanesulfinyl-pyrimidin-4-yl)-2-methyl-imidazo[1,2b]pyridazine (method A, stage A.4.) (45 mg; 0.075 mMol) dissolved in dioxane (2 mL) is added imidazole (15.3 mg; 0.224 mMol) and the mixture kept stirring at 75° C. for 2 h. After this the mixture is heated in a microwave oven for 1 h at 100° C., followed by 30 min at 150° C., then 7 h at 170° C. to complete the reaction. After removal of the solvent under reduced pressure, the residue is taken up into CH₂Cl₂ (30 mL), washed with NaHCO₃ (satd. soln.; 20 mL) and brine (20 mL). The combined organics are dried over Na₂SO₄, filtered off and freed from the solvent under reduced pressure. Purification is done by chromatography on silica gel (Redisep 12 g; CH₂Cl₂/CH₃OH/NH₄OH (32%) 98:2.0.2) to obtain the title compound (25.5 mg) as yellow powder. Title compound: MS (ESI⁺):m/z=414.1 (M+H)⁺; HPLC: tRet=3.850 minutes.

### Stage A.1: 4-Methyl-2-methylsulfanyl-pyrimidine (A.1.)

4-Methyl-2-methylsulfanyl-pyrimidine (14 g; 94.9 mMol) is dissolvedin THF anhydrous under argon and colled to −78° C. Within 60 min. LDA (2 M soln in hexane; 71 mL; 140 mMol) is added dropwise while keeping the temperature <−75° C. Stirring is continued for 3 h followed by addition of N-methoxy-N-methylacetamide (10.0 g; 94.9 mMol) at −75° C. After this, cooling is removed and the mixture is allowed to stir at RT for 3 h. After removal of the solvent under reduced pressure, the residue is taken up into CH₂Cl₂ (250 mL), washed with water (100 mL) and brine (100 mL). The combined organics are dried over Na₂SO₄, filtered off and freed from the solvent under reduced pressure. Purification is done by chromatography on silica gel (Redisep 40 g; hexane/EtOAc 3/1) to obtain the title compound (4.29 g) as yellow oil. Title compound: MS (ESI⁺):m/z=183.1 (M+H)⁺; HPLC: tRet=3.714 minutes.

### Stage A2: 1-Chloro-1-(2-methylsulfanyl-pyrimidin-4-yl)-propan-2-one (A.2.)

4-Methyl-2-methylsulfanyl-pyrimidine (A.1.) (method A, stage A.1.) (0.8 g; 4.39 mMol) is dissolved in CH₂Cl₂ (10 mL) under an atmosphere of argon and cooled to 0°-4° C. Within 60 min a solution of sulfurylchloride (0.431 mikroL; 5.26 mMol) dissolved in CH₂Cl₂ (10 mL) is added dropwise and stirring is continued at 0° C. for 19 h. After this, sulfurylchloride (0.107 mikroL; 1.31 mMol) is added and stirring continued for another 1.5 h. To this reaction mixture, water (22 mL, cold) is added dropwise, followed by addition of CH₂Cl₂. The organic layer is extracted with water (1×, cold) and brine. The aqueous layers are back extracted with water (2×, cold) and the combined organics are dried over Na₂SO₄, filtered off and freed from the solvent under reduced pressure. The crude product (yellow oil; 850 mg) is used in the next step without further purification. Title compound: MS (ESI⁺):m/z=217.1 (M+H)⁺; HPLC: tRet=6.248 minutes.

### Stage A.3.: 6-(3,4-Dimethoxy-phenyl)-2-methyl-3-(2-methylsulfanyl-pyrimidin-4-yl)-imidazo[1,2-b]pyridazine (A.3.)

6-(3,4-Dimethoxy-phenyl)-pyridazin-3-ylamine (example 20; stage 20.2) (410 mg; 1.70 mMol) and 1-Chloro-1-(2-methylsulfanyl-pyrimidin-4-yl)-propan-2-one (method A, stage A.2.) (639 mg; 2.55 mMol) is dissolved DMA (12 mL) under an atmosphere of argon followed by addition of Et3N (0.538 mL; 3.82 mMol) and the mixture is then heated under stirring at 170° C. in a microwave oven. After cooling to RT, the mixture is freed from the solvent under reduced pressure.

The residue is taken up into CH₂Cl₂, washed with water (2×) and brine (1×). The combined organics are dried over Na₂SO₄, filtered off and freed from the solvent under reduced pressure. Purification is done by chromatography on silica gel (Redisep 40 g; eluting with EtOAc) to obtain the title compound (0.385 g) as yellow oil. Title compound: MS (ESI⁴):m/z=394.1 (M+H)⁺; HPLC: tRet=5.218 minutes.

### Stage A.4.: 6-(3,4-Dimethoxy-phenyl)-3-(2-methanesulfinyl-pyrimidin-4-yl)-2-methyl-imidazo[1,2b] pyridazine (A.4.)

6-(3,4-Dimethoxy-phenyl)-2-methyl-3-(2-methylsulfanyl-pyrimidin-4-yl)-imidazo[1,2-b]pyridazine (method A, stage A.3.) (374 mg; 0.644 mMol) is dissolved in CH₂Cl₂ (25 mL) under an atmosphere of argon and cooled to 0°-4° C. Within 15 min, 3-chloroperbenzoic acid (278 mg; 1.128 mMol) is added portion wise and the mixture is kept stirring at 0° C. for 90 min.

The reaction mixture is diluted with water and extracted with CH₂Cl₂, washed with water (2×) and brine (1×). The combined organics are dried over Na₂SO₄, filtered off and freed from the solvent under reduced pressure. Purification is done by chromatography on silica gel (Redisep 1 g; eluting with CH₂Cl₂/EtOAc) to obtain the title compound (0.189 g) as a beige foam. Title compound: MS (ESI⁺):m/z=410.1 (M+H)⁺; HPLC: tRet=3.795 minutes.

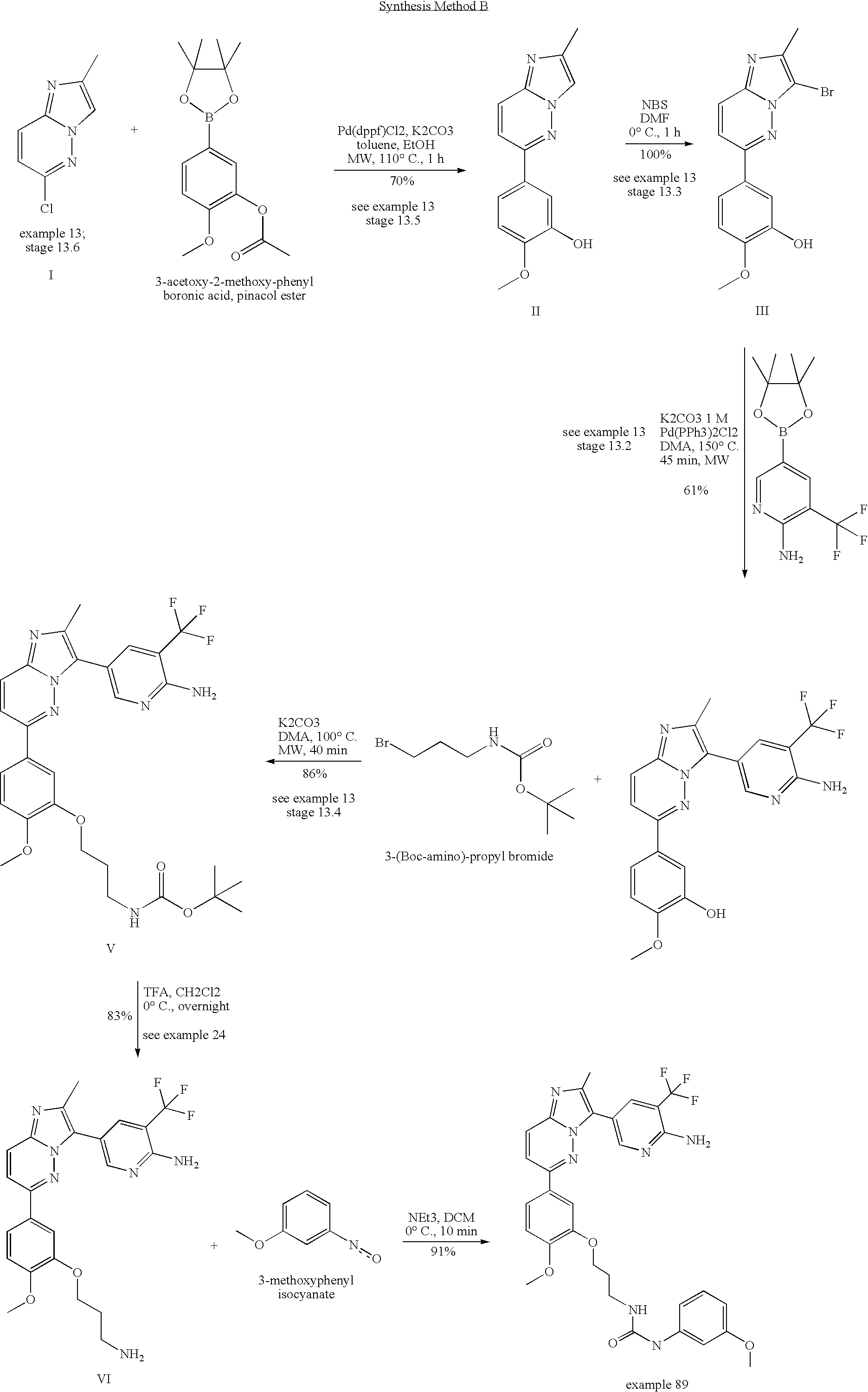

### Example 89

### 1-(3-{5-[3-(6-Amino-5-trifluoromethyl-pyridin-3-yl)-2-methyl-imidazo[1,2-b]pyridazin-6-yl]-2-methoxy-phenoxy}-propyl)-3-(3-methoxy-phenyl)-urea

To a solution of 5-{6-[3-(3-Amino-propoxy)-4-methoxy-phenyl]-2-methyl-imidazo[1,2-b]pyridazin-3-yl}-3-trifluoromethyl-pyridin-2-ylamine (VI) (35 mg; 0.074 mMol), triethylamine

(10.8 mikroL; 0.0778 mMol) in DMF (1 mL) at 0° C. is added 3-methoxyphenyl isocyanate

(9.46 mikroL; 0.0733 mMol) and the mixture is kept stirring for 10 min at 0° C. NaHCO₃ (satd. soln., 2 mL) is added and stirring continued for another 10 min at 0° C. The reaction mixture is extracted with CH₂Cl₂ and a few drops of EtOH (2×). The combined organic layers are dried over Na₂SO₄, filtered off and freed from the solvent under reduced pressure. Purification is done by chromatography on silica gel (Redisep 4 g; eluting with CH₂Cl₂/EtOAc/MeOH from 100/0/0 to 90/8/2) to obtain the title compound (42 mg) as a yellow powder. Title compound: MS (ESI⁺):m/z=622 (M+H)⁺; HPLC: tRet=4.691 minutes.

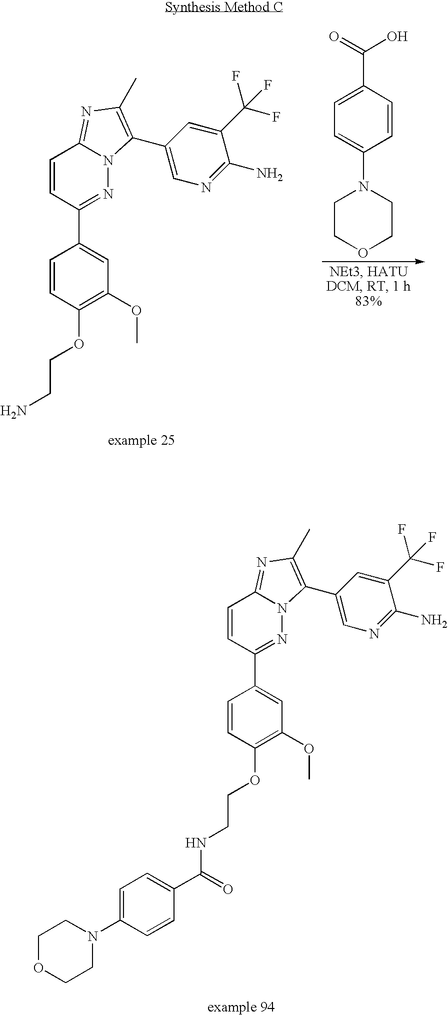

### Example 94

### 1-(3-{5-[3-(6-Amino-5-trifluoromethyl-pyridin-3-yl)-2-methyl-imidazo[1,2-b]pyridazin-6-yl]-2-methoxy-phenoxy}-propyl)-3-(3-methoxy-phenyl)-urea

A solution of HATU (34 mg; 0.089 mMol), triethylamine (30 mikroL) and 4-morpholine-4-yl-benzoic acid (27 mg; 0.13 mMol) in CH₂Cl₂ (2 mL) is stirred at RT for 10 min. Then a solution of 5-{6-[4-(2-Amino-ethoxy)-3-methoxy-phenyl]-2-methyl-imidazo[1,2-b]pyridazin-3-yl}-3-trifluoromethyl-pyridin-2-ylamine (example 25) (40 mg; 0.087 mMol) in CH₂Cl₂ (2 mL) is added and the mixture kept stirring at RT for 1 h. The reaction mixture is extracted with CH₂Cl₂/MeOH (9/1) and water. The aqueous layer is washed with CH₂Cl₂. The combined organic layers are dried over Na₂SO₄, filtered off and freed from the solvent under reduced pressure. Purification is done by chromatography on silica gel (Redisep 4 g; eluting with CH₂Cl₂/EtOAc/MeOH from 100/0/0 to 30/24/6) to obtain the title compound (47 mg) as a yellow powder. Title compound: MS (ESI⁺): m/z=648 (M+H)⁺; HPLC: tRet=4.327 minutes.

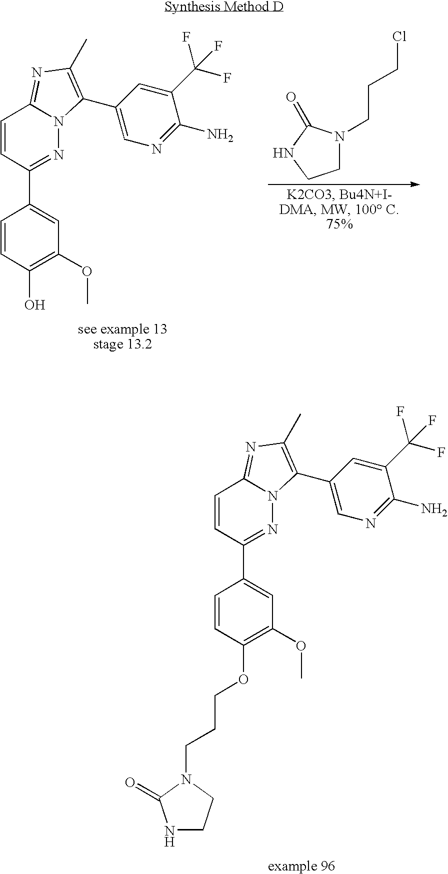

### Example 96

### 1-(3-{4-[3-(6-Amino-5-trifluoromethyl-pyridin-3-yl)-2-methyl-imidazo[1,2-b]pyridazin-6-yl]-2-methoxy-phenoxy}-propyl)-imidazolidin-2-one

A mixture of 4-[3-(6-Amino-5-trifluoromethyl-pyridin-3-yl)-2-methyl-imidazo[1,2-b]pyridazin-6-yl]-2-methoxy-phenol (see example 13; stage 13.2) (50 mg; 0.12 mMol), 1-(3-chloropropyl)-imidazolidinone (20 mg; 0.123 mMol), K₂CO₃ (18 mg; 0.130 mMol) and tetrabutylammonium iodide (4 mg; 0.011 mMol) is dissolved in DMA (3 mL) and stirred for 1 h at 120° C. in the microwave oven, followed by 90 min at 120° C. The reaction mixture is extracted with CH₂Cl₂ and water. The aqueous layer is washed with CH₂Cl₂. The combined organic layers are dried over Na₂SO₄, filtered off and freed from the solvent under reduced pressure. Purification is done by chromatography on silica gel (Redisep 4 g; eluting with CH₂Cl₂/EtOAc/MeOH from 100/0/0 to 0/80/20) to obtain the title compound (49 mg) as a powder. Title compound: MS (ESI⁺):m/z=542 (M+H)⁺; HPLC: tRet=3.865 minutes.

### Analytical HPLC Conditions:

### System 2

Linear gradient 2-100% CH₃CN (0.1% TFA) and H₂O (0.1% TFA) in 7 min+2 min 100% CH₃CN (0.1% TFA); detection at 215 nm, flow rate 1 mL/min at 30° C. Column: Nucleosil 100-3 C18HD (125×4 mm)

### Example 110

### Cyclopropanecarboxylic acid (3-{4-[3-(6-amino-5-trifluoromethyl-pyridin-3-yl)-2-methyl-imidazo[1,2-b]pyridazin-6-yl]-phenoxymethyl}-oxetan-3-yl)-amide

In a 5 ml vial with magnetic stir bar 50 mg (0.064 mmol) of 5-{6-[4-(3-Amino-oxetan-3-ylmethoxy)-phenyl]-2-methyl-imidazo[1,2-b]pyridazin-3-yl}-3-trifluoromethyl-pyridin-2-ylamine (for preparation see example 111) and 22.4 μL (0.159 mmol) of triethylamine are dissolved in 1 mL of CH2Cl2 under nitrogen. Thereafter a solution of 5.9 μL (0.064 mmol) of cyclopropanecarbonyl chloride in 0.5 mL of CH2Cl2 is added slowly at room temperature. After complete addition, no more starting material can be detected in the HPLC or MS. The reaction mixture is filtered and the solvent is evaporated. The crude product is purified by preparative HPLC. Fractions containing pure product are treated with soda and then the solvent is evaporated. The residue is partitioned between ethyl acetate and sodium bicarbonate solution and the organic phase is washed with brine, dried with sodium sulfate and evaporated to yield the title compound as a yellow solid. MS-ES: (M+1)=539.1, HPLC: t_{R}=4.934 min. (System 2) M.p. 191-193° C.

### Example 111

### 5-{6-[4-(3-Amino-oxetan-3-ylmethoxy)-phenyl]-2-methyl-imidazo[1,2-b]pyridazin-3-yl}-3-trifluoromethyl-pyridin-2-ylamine

Crude (3-{-4-[3-(6-amino-5-trifluoromethyl-pyridin-3-yl)-2-methyl-imidazo[1,2-b]pyridazin-6-yl]-phenoxymethyl}-oxetan-3-yl)-carbamic acid benzyl ester (for preparation see stage 111.1), 0.67 g (˜0.94 mmol) is deprotected by hydrogenation at 5 bar and room at temperature with 10% Palladium on carbon (0.15 g) in 30 mL of THF. After 18 hours the hydrogenation is stopped and the catalyst filtered off through a pad of hyflo. The solvent is evaporated, the residue is taken up in CH2Cl2 and extracted with 2 N hydrochloric acid. The organic phase is re-extracted with water and the aqueous phase is washed with CH2Cl2. Thereafter the pH of the combined aqueous extracts is adjusted to ˜10 by the addition of sodium hydroxide solution. Extraction with CH2Cl2 (3×) followed by drying over Na₂SO₄, and evaporation of the solvent gives the title compound as a yellow solid. MS-ES.: (M+1) 471.1, HPLC: t_{R}=4.177 min. (System 2) M.p. 198-200° C.

### Stage 111.1 Example 112 (3-{4-[3-(6-Amino-5-trifluoromethyl-pyridin-3-yl)-2-methyl-imidazo[1,2-b]pyridazin-6-yl]-phenoxymethyl}-oxetan-3-yl)-carbamic acid benzyl ester

A 100 mL flask containing a mixture of 1.5 g (90%, ˜3.07 mmol) {3-[4-(4,4,5,5-tetramethyl-[1,3,2]dioxaborolan-2-yl)-phenoxymethyl]-oxetan-3-yl}-carbamic acid benzyl ester (for preparation see stage 111.2), 1.17 g (95%, 3.39 mmol) 5-(6-chloro-2-methyl-imidazo[1,2-b]pyridazin-3-yl)-3-trifluoromethyl-pyridin-2-ylamine, 138 mg (98%, 0.166 mmol) PdCl2(dppf), 1.52 g (11.0 mmol) potassium carbonate, 20 mL ethanol and 40 mL of toluene is purged with nitrogen. The mixture is then heated under reflux for 8 hours. Only traces of starting material can be detected in the HPLC after this time. The reaction mixture is filtered through a pad of hyflo and the solvent is evaporated. Trituration of the residue with ethyl acetate and filtration gives the title compound as a yellow solid. MS: (M+1)=605.0; HPLC: t_{R}=5.669 min. (System 2) M.p. 209-212° C. Rf (CH2Cl2/EtOH 95:5)=0.4.

### Stage 111.2 {3-[4-(4,4,5,5-tetramethyl-[1,3,2]dioxaborolan-2-yl)-phenoxymethyl]-oxetan-3-yl}-carbamic acid benzyl ester

A 250 mL flask containing a mixture of 3.1 g (95%, 7.51 mmol) [3-(4-bromo-phenoxymethyl)-oxetan-3-yl]-carbamic acid benzyl ester (for preparation see stage 111.3), 2.16 g (8.25 mmol) bis-(pinacolato)diboron, 271 mg (98%, 0.368) Pd(PPh3)2Cl2, 1.55 g (15.8 mmol) potassium acetate and 80 mL of toluene is purged with nitrogen. The mixture is then heated under reflux for 16 hours. No starting material can be detected in the HPLC and MS after this time. The reaction mixture is filtered through a pad of hyflo and the solvent is evaporated. The brown residue is purified by chromatography on 40 g of silica gel on a Combiflash Companion (Isco Inc.) using a gradient of hexanes/ethyl acetate from 9:1 to 8:2. Pure fractions are combined and the solvent is evaporated to leave the title compound as a colorless resin. MS: (M+1)=440.0; HPLC: t_{R}=4.703 min. (System 2) Rf (hexanes/EtOAc 2:1)=0.5.

### Stage 111.3 [3-(4-Bromo-phenoxymethyl)-oxetan-3-yl]-carbamic acid benzyl ester

A mixture of 2 g (6.83 mmol) 3-(4-bromo-phenoxymethyl)-oxetane-3-carboxylic acid (for preparation see stage 111.4), 0.79 mL (7.5 mmol) benzyl alcohol, 1.8 mL (˜90%, 7.49 mmol) DPPA, 1.06 mL (7.5 mmol) triethylamine and 75 mL of toluene in a 250 mL flask is heated to 100° C. under nitrogen for 5 hours. Only traces of starting material can be detected in the HPLC after this time. After cooling the reaction mixture is washed with NaHCO3 solution. The aqueous phase is extracted with toluene and the combined organic layers are washed with brine and dried with Na2SO4. Evaporation of the solvent gave an oil which is purified by chromatography on 80 g of silica gel on a Combiflash Companion (Isco Inc.) using a gradient of hexanes/ethyl acetate from 9:1 to 8:2. Pure fractions are combined and the solvent is evaporated to leave the title compound as a colorless solid. MS: (M+1)=392.0/393.9; HPLC: t_{R}=7.081 min. (System 2) Rf (hexanes/EtOAc 2:1)=0.4; M.p. 101-103° C.

### Stage 111.4 3-(4-Bromo-phenoxymethyl)-oxetane-3-carboxylic acid

In a 500 mL three-necked flask equipped with a condenser, stir bar and nitrogen inlet are placed 6 g (95%, 20.9 mmol) of [3-(4-bromo-phenoxymethyl)-oxetan-3-yl]-methanol (for preparation see stage 111.5), 0.333 g (2.09 mmol) TEMPO, 240 mL acetonitrile and 120 mL of phosphate buffer (pH 7). A solution of 5.6 g (49.5 mmol) NaClO2 (sodium chlorite), 0.72 mL (1.04 mmol) of a 11% sodium hypochlorite solution and 30 mL of water is then added at RT and the mixture is heated at 77° C. for 20 hours. After cooling the acetonitrile is evaporated and the aqueous residue washed with ethyl acetate, acidified with 2N HCl and extracted with ethyl acetate. The organic extracts are washed with brine, dried with Na2SO4 and evaporated to give a colorless residue. The first ethyl acetate washings are extracted with NaHCO3 solution and the aqueous phase is acidified with 2N HCl. This equeous phase is then extracted with CH2Cl2 and the organic phase washed with brine, dried with Na2SO4 and evaporated to give a colorless solid. According to HPLC analysis both residues are identical. They are re-dissolved, combined and the solvent is evaporated to give the title compound as a colorless solid. MS: (M+1)=285/287.2; HPLC: t_{R}=5.845 min. (System 2); M.p. 122-124° C.

### Stage 111.5 [3-(4-Bromo-phenoxymethyl)-oxetan-3-yl]-methanol

In a 250 mL three-necked flask equipped with a condenser, stir bar and nitrogen inlet are placed 7.5 g (62.2 mmol) (3-hydroxymethyl-oxetan-3-yl)-methanol (for preparation see stage 111.6), 11 g (62.3 mmol) 4-bromophenol, 16.7 g (62.2 mmol) triphenylphosphin and 120 mL of THF. Thereafter, 12.3 mL (62.2 mmol) diisopropyl azodicarboxylate is added dropwise within 1.5 hours (slightly exothermic). After stirring the solution at RT for 4 hours 1 mL of diisopropyl azodicarboxylate is added (5 minutes) and the solution stirred for one more hour. The THF is then evaporated and the resulting yellow oil is taken up in ethyl acetate and treated with hexanes. After stirring for 10 minutes the precipitate is filtered off and discarded and the filtrate is concentrated to a yellow oil. This is purified by chromatography on 80 g of silica gel on a Combiflash Companion (Isco Inc.) using a gradient of CH2Cl2/EtOAc 9:1 to 1:1. Enriched fractions are combined, evaporated and re-chromatographed on 80 g of silica gel on a Combiflash Companion (Isco Inc.) using a gradient of hexanes/EtOAc from 85:15 to 75:25. Pure fractions are combined and the solvent is evaporated to leave the title compound as a colorless solid. MS: (M−1)=271/273; HPLC: t_{R}=5.77 min. (System 2).

### Stage 111.6 (3-Hydroxymethyl-oxetan-3-yl)-methanol

A 1 L flask is charged with 100 g (0.727 mol) 2-bis-hydroxymethyl-propane-1,3-diol (pentaerythritol, ABCR), 115 mL (0.92 mol) diethyl carbonate and 13 mL EtOH. Powdered potassium hydroxide, 237 mg (3.63 mmol), is added and the mixture heated under reflux for 4 hours. After addition of another portion of 230 mg of potassium hydroxide the reflux condenser is replaced and EtOH is distilled out of the reaction mixture (bath temperature ˜135° C.). Within 4 hours 90 mL of ethanol are collected. The condenser is replaced again by a solid trap the apparatus is connected to a vacuum pump and the mixture is gradually heated to 240° C. at 0.5 to 1 mbar. The title compound is collected as a colorless solid. MS: (M+1)=119.0; Rf (EtOAc/EtOH 9:1)=0.3.

### Example 113

### (3-{4-[3-(6-Amino-5-trifluoromethyl-pyridin-3-yl)-2-methyl-imidazo[1,2-b]pyridazin-6-yl]-phenoxymethyl}-oxetan-3-yl)-carbamic acid tert-butyl ester

The title compound is prepared in analogy to the compound prepared in stage 111.1. MS: (M+1)=571.1; HPLC: t_{R}=5.569 min. (System 2). Rf (CH2Cl2:EtOH 95:5)=0.31. The starting material is prepared according to stage 113.1.

### Stage 113.1 {3-[4-(4,4,5,5-Tetramethyl-[1,3,2]dioxaborolan-2-yl)-phenoxymethyl]-oxetan-3-yl}-carbamic acid tert-butyl ester

The title compound is prepared in analogy to the compound prepared in stage 111.2. MS: (M+1)=406.1; HPLC: t_{R}=7.427 min. (System 2). Rf (hexanes/EtOAc)=0.41. The starting material is prepared according to stage 113.2.

### Stage 113.2 [3-(4-Bromo-phenoxymethyl)-oxetan-3-yl]-carbamic acid tert-butyl ester

The title compound is prepared in analogy to the compound prepared in stage 111.3. MS: (M+1)=358.1/360; HPLC: t_{R}=7.074 min. (System 2).

The example compounds in the following table are prepared in analogy to the compound prepared in Example 110:

| Example | Product | data |
|---|---|---|
| 114 | 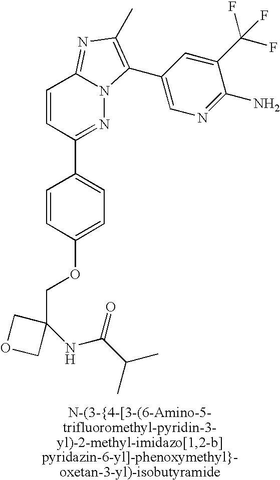 | MS: (M + 1) = 541.4, HPLC: t_{R} = 5.014 (System 2) min., Rf (CH2Cl2/EtOH 9: 1) = 0.22 |
| 115 | 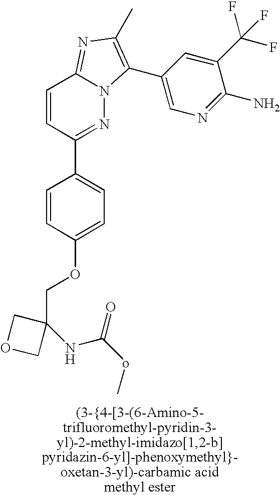 | MS: (M + 1) = 529.3, HPLC: t_{R} = 4.932 min. (System 2), Rf (CH2Cl2/EtOH 95: 5) = 0.34, M. p. 202-204° C. |
| 116 | 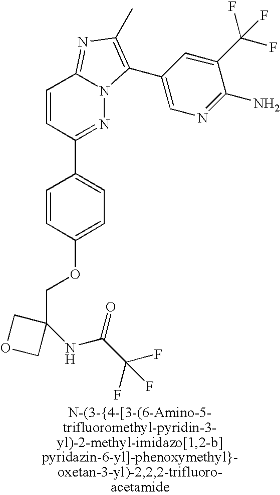 | MS: (M + 1) = 567.0, HPLC: t_{R} = 5.319 min. (System 2), Rf (CH2Cl2/EtOH 95: 5) = 0.35, M. p. 214-216° C. |

Biological Data

| | Enzymatic Data | | | |
|---|---|---|---|---|
| | | | | PI3K |
| | | PI3K beta | PI3K delta | gamma |
| EXAMPLE | PI3K alfa | IC50 | IC50 | IC50 |
| NUMBER | IC50 [umol] | [umol] | [umol] | [umol] |
| 1 | 0.206 | 1.682 | 1.353 | 0.241 |
| 2 | 0.059 | 1.371 | 0.309 | |
| 3 | 0.0964 | 2.310 | 0.514 | |
| 4 | 0.044 | 1.212 | 0.152 | 0.345 |
| 5 | 0.111 | 1.571 | 1.090 | 0.613 |
| 6 | 0.455 | 4.194 | 2.710 | 1.848 |
| 7 | 0.096 | 0.976 | 0.694 | 0.419 |
| 8 | 0.042 | 1.170 | 0.456 | 0.303 |
| 11 | 0.110 | 0.613 | 2.392 | 0.536 |
| 13 | 0.058 | 4.158 | >25 | >25 |
| 14 | 0.138 | 5.152 | 2.049 | 0.688 |
| 15 | 0.0059 | 0.150 | 0.069 | 0.157 |
| 16 | >25 | >25 | >25 | >25 |
| 17 | 0.181 | 4.476 | 2.022 | 0.797 |
| 18 | >25 | 5.160 | >25 | >25 |
| 19 | 1.286 | 12.277 | 5.501 | 5.184 |
| 20 | 0.22 | 3.244 | 1.388 | 0.358 |
| 21 | 0.408 | >9.1 | 0.406 | 0.655 |
| 22 | 0.781 | >9.1 | 0.889 | 1.502 |
| 23 | 0.136 | >9.1 | 0.124 | 0.511 |
| 24 | 0.039 | 0.051 | 0.052 | 0.211 |
| 25 | 0.015 | 0.193 | 0.024 | 0.507 |
| 27 | 0.456 | >9.100 | >9.100 | 2.281 |
| 29 | 0.093 | >9.100 | 1.588 | 2.741 |
| 31 | 0.038 | >9.100 | 0.748 | 4.324 |
| 32 | 3.561 | >9.100 | >9.100 | >9.100 |
| 37 | 0.233 | >9.100 | >9.100 | 2.300 |
| 39 | 0.368 | >9.100 | >9.100 | >9.100 |
| 41 | 0.294 | >9.100 | >9.100 | 0.933 |
| 47 | 1.812 | >9.100 | >9.100 | 4.501 |
| 51 | 0.028 | >9.100 | 1.664 | 2.515 |
| 55 | 3.688 | >9.100 | >9.100 | >9.100 |
| 58 | 0.925 | >9.100 | >9.100 | >9.100 |
| 59 | 0.371 | >9.100 | >9.100 | 2.643 |
| 61 | 0.242 | >9.100 | 2.714 | 0.194 |
| 77 | 0.075 | 3.793 | 0.281 | 1.261 |
| 79 | 0.099 | >9.1 | 2.045 | 0.648 |
| 80 | 0.175 | >9.1 | 2.704 | 1.932 |
| 81 | 0.030 | >9.1 | 0.372 | 0.503 |
| 82 | 0.019 | 4.008 | 0.164 | 0.087 |
| 83 | 0.276 | >9.100 | 0.397 | 1.162 |
| 84 | 0.040 | >9.100 | 0.174 | 0.065 |
| 85 | 0.244 | >9.100 | 3.490 | 2.587 |
| 86 | 2.149 | >9.100 | 3.995 | 8.511 |
| 87 | 0.296 | >9.100 | 0.357 | 0.098 |
| 88 | 0.051 | 3.032 | 0.041 | 0.098 |
| 89 | 1.640 | >9.100 | >9.100 | 6.856 |
| 90 | 2.363 | >9.100 | 1.740 | 3.462 |
| 91 | 4.105 | >9.100 | 3.398 | >9.100 |
| 92 | 1.780 | >9.100 | >9.100 | >9.100 |
| 93 | 2.325 | >9.100 | 4.132 | 3.306 |
| 94 | 0.367 | >9.100 | 6.776 | >9.100 |
| 95 | 0.245 | >9.100 | 5.368 | 4.739 |
| 96 | 0.031 | 4.862 | 0.424 | 0.805 |
| 97 | 0.118 | >9.100 | 0.966 | 1.332 |
| 98 | 9.087 | >9.100 | >9.100 | >9.100 |
| 99 | >9.1 | >9.100 | >9.100 | >9.100 |
| 100 | 0.063 | 1.039 | 0.153 | 1.176 |
| 101 | 1.044 | >9.100 | 1.183 | >9.100 |
| 102 | 2.988 | >9.100 | 2.562 | >9.100 |
| 103 | 0.015 | 1.829 | 0.362 | 0.662 |
| 104 | 0.031 | 2.080 | 0.255 | 0.477 |
| 105 | 0.083 | 6.257 | 0.273 | 0.518 |
| 106 | 2.205 | >9.100 | 1.045 | 1.055 |
| 107 | 4.686 | >9.100 | 2.986 | >9.100 |
| 108 | 0.985 | 7.083 | 2.274 | 6.792 |
| 109 | 5.911 | >9.100 | 3.943 | >9.100 |
| 110 | 0.126 | 8.190 | 1.594 | 2.076 |
| 111 | 0.131 | 2.389 | 0.895 | 1.190 |
| 112 | 0.821 | >9.100 | 1.816 | >9.100 |
| 113 | 1.008 | >9.100 | 5.108 | >9.100 |
| 114 | 0.049 | 8.732 | 0.435 | 1.524 |
| 115 | 0.295 | >9.100 | 1.970 | 3.162 |
| 116 | 0.059 | 4.239 | 0.937 | 4.665 |

### Example 117

### Soft Capsules

5000 soft gelatin capsules, each comprising as active ingredient 0.05 g of one of the compounds of formula I mentioned in the preceding Examples, are prepared as follows:

Composition

| | |
|---|---|
| Active ingredient | 250 g |
| Lauroglycol | 2 litres |

Preparation process: The pulverized active ingredient is suspended in Lauroglykol® (propylene glycol laurate, Gattefossa S.A., Saint Priest, France) and ground in a wet pulverizer to produce a particle size of about 1 to 3 μm. 0.419 g portions of the mixture are then introduced into soft gelatin capsules using a capsule-filling machine.

### Example 118

### Tablets Comprising Compounds of the Formula I

Tablets, comprising, as active ingredient, 100 mg of any one of the compounds of formula I of Examples 1 to 116 are prepared with the following composition, following standard procedures:

Composition

| | |
|---|---|
| Active Ingredient | 100 mg |
| crystalline lactose | 240 mg |
| Avicel | 80 mg |
| PVPPXL | 20 mg |
| Aerosil | 2 mg |
| magnesium stearate | 5 mg |
| | 447 mg |

Manufacture: The active ingredient is mixed with the carrier materials and compressed by means of a tabletting machine (Korsch EKO, Stempeldurchmesser 10 mm).

Avicel® is microcrystalline cellulose (FMC, Philadelphia, USA). PVPPXL is polyvinylpolypyrrolidone, cross-linked (BASF, Germany). Aerosil® is silicium dioxide (Degussa, Germany).

1. A compound of the formula I, 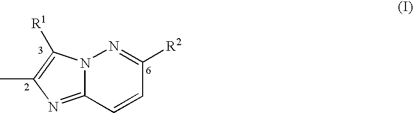
wherein
R¹ is unsubstituted or substituted aryl or heterocyclyl;
and
R² is substituted phenyl or substituted naphthyl;
and/or an N-oxide thereof, a solvate and/or a salt thereof.

2. A compound of the formula I according to claim 1, wherein
R¹ is unsubstituted or substituted aryl or heterocyclyl wherein,
aryl has 6 to 18 carbon atoms and is a mono-, di- or tricyclic unsaturated carbocyclic moiety with conjugated double bonds in the ring, each of which is unsubstituted or substituted by one to three substituents independently selected from the group consisting C₁-C₇-alkyl;
C₂-C₇-alkenyl;
C₂-C₇-alkinyl;
C₆-C₁₈-aryl-C₁-C₇-alkyl in which aryl is unsubstituted or substituted by C₁-C₇-alkyl, pyrrolidinyl, piperazinyl, amino, N-mono- or N,N-di-C₁-C₇-alkylamino, halo, hydroxy, C₁-C₇-alkoxy, and/or halo-C₁-C₇-alkyl;
Heterocyclyl-C₁-C₇-alkyl, in which heterocyclyl is pyrrolidinyl, piperidinyl, piperazinyl, morpholino, thiomorpholino, pyridinyl, pyrimidinyl, pyrazinyl, pyridazinyl, oxazolyl or thiazolyl and wherein the heterocyclyl is unsubstituted or substituted by C₁-C₇-alkyl, pyrrolidinyl, piperazinyl, amino, N-mono- and N,N-di-C₁-C₇-alkylamino, halo, hydroxyl, C₁-C₇-alkoxy, oxo and/or halo-C₁-C₇-alkyl;
Heterocyclyl-oxy-C₁-C₇-alkyl, in which heterocyclyl is pyrrolidinyl, piperidinyl, piperazinyl, morpholino, thiomorpholino, pyridinyl, pyrimidinyl, pyrazinyl, pyridazinyl, oxazolyl or thiazolyl and wherein heterocyclyl is unsubstituted or substituted by C₁-C₇-alkyl, pyrrolidinyl, piperazinyl, amino, N-mono- and N,N-di-C₁-C₇-alkylamino, halo, hydroxyl, C₁-C₇-alkoxy, oxo and/or halo-C₁-C₇-alkyl;
Heterocyclyl-carbonyl-C₁-C₇-alkyl, in which heterocyclyl is pyrrolidinyl, piperidinyl, piperazinyl, morpholino, thiomorpholino, pyridinyl, pyrimidinyl, pyrazinyl, pyridazinyl, oxazolyl or thiazolyl wherein the heterocycyl is unsubstituted or substituted by C₁-C₇-alkyl, pyrrolidinyl, piperazinyl, amino, N-mono- and N,N-di-C₁-C₇-alkylamino, halo, hydroxyl, C₁-C₇-alkoxy, oxo and/or halo-C₁-C₇-alkyl;
halo-C₁-C₇-alkyl;
hydroxy-C₁-C₇-alkyl;
C₁-C₇-alkoxy-C₁-C₇-alkyl;
C₁-C₇-alkoxy-C₁-C₇-alkoxy-C₁-C₇-alkyl;
phenyloxy-C₁-C₇-alkyl- or naphthyloxy-C₁-C₇-alkyl;
phenyl-C₁-C₇-alkoxy-C₁-C₇-alkyl or naphthyl-C₁-C₇-alkoxy-C₁-C₇-alkyl;
amino-C₁-C₇-alkyl;
N-mono- or N,N-di-(C₁-C₇-alkyl, C₁-C₇-alkoxy-C₁-C₇-alkyl and/or (mono- or di-(C₁-C₇-alkyl)-amino)-C₁-C₇-alkyl)-amino-C₁-C₇-alkyl;
C₁-C₇-alkoxy-C₁-C₇-alkylamino-C₁-C₇-alkyl;
mono- or di-[C₆-C₁₈-aryl]-C₁-C₇-alkyl in which aryl is preferably phenyl, naphthyl, biphenylenyl, indacenyl, acenaphthylenyl, fluorenyl, phenalenyl, phenanthrenyl or anthracenyl and unsubstituted or substituted by C₁-C₇-alkyl, such as methyl or ethyl, by pyrrolidinyl, especially pyrrolidino, by piperazinyl, especially piperazino, by amino, by N-mono- and/or N,N-di-C₁-C₇-alkylamino, by halo, by hydroxyl, by C₁-C₇-alkoxy, such as methoxy, and/or by halo-C₁-C₇-alkyl, such as trifluoromethyl;
(naphthyl- or phenyl-C₁-C₇-alkyl)-amino-C₁-C₇-alkyl;
C₁-C₇-alkanoylamino-C₁-C₇-alkyl;
carboxy-C₁-C₇-alkyl;
benzoyl- or naphthoylamino-C₁-C₇-alkyl;
C₁-C₇-alkylsulfonylamino-C₁-C₇-alkyl;
phenyl- or naphthylsulfonylamino-C₁-C₇-alkyl wherein phenyl or naphthyl is unsubstituted or substituted by one or more, especially one to three, C₁-C₇-alkyl moieties;
phenyl- or naphthyl-C₁-C₇-alkylsulfonylamino-C₁-C₇-alkyl;
cyano-C₁-C₇-alkyl;
halo, especially fluoro (preferred), chloro (preferred) or bromo;
hydroxy;
C₁-C₇-alkoxy;
C₆-C₁₈-aryl-C₁-C₇-alkoxy in which aryl is phenyl or naphthyl and each aryl is unsubstituted or substituted by C₁-C₇-alkyl, C₁-C₇-alkoxy, pyrrolidinyl, piperazinyl, amino, N-mono- and/or N,N-di-C₁-C₇-alkylamino, halo, hydroxyl, C₁-C₇-alkoxy, and/or by halo-C₁-C₇-alkyl;
hydroxy-C₁-C₇-alkoxy;
C₁-C₇-alkoxy-C₁-C₇-alkoxy;
C₁-C₇-alkoxy-C₁-C₇-alkoxy-C₁-C₇-alkoxy;
halo-C₁-C₇-alkoxy;
amino-C₁-C₇-alkoxy;
N-mono- or N,N-di-(C₁-C₇-alkyl)-amino-C₁-C₇-alkoxy;
N—C₁-C₇-alkanoylamino-C₁-C₇-alkoxy;
C₁-C₇-alkoxycarbonylamino-C₁-C₇-alkoxy;
C₆-C₁₄-arylcarbonylamino-C₂-C₇-alkoxy wherein aryl is unsubstituted or substituted by one to three substituents independently selected from the group consisting of C₁-C₇-alkyl, halo-C₁-C₇-alkyl, hydroxy, C₁-C₇-alkoxy, halo and cyano;
N-unsubstituted-, N-mono- or N,N-di-(C₁-C₇-alkyl)carbamoyl-C₁-C₇-alkoxy;
phenyloxy- or naphthyloxy;
phenyl-C₁-C₇-alkyloxy or naphthyl-C₁-C₇-alkyloxy;
heterocyclyl-C₁-C₇-alkoxy in which the heterocyclyl is pyrrolyl, pyrrolidinyl, imidazolyl, imidazolidinyl piperidinyl piperazinyl, pyridinyl, pyrimidinyl, pyrazinyl, pyridazinyl, oxazolyl, thiazolyl, morpholinyl, thiomorpholinyl, S-oxothiomorpholinyl or S,S-dioxothiomorpholinyl wherein the heterocyclyl is unsubstituted or substituted by C₁-C₇-alkyl, pyrrolidinyl, piperazinyl, amino, N-mono- and/or N,N-di-C₁-C₇-alkylamino, halo, hydroxyl, C₁-C₇-alkoxy, oxo and/or halo-C₁-C₇-alkyl;
heterocyclyl-oxy-C₁-C₇-alkoxy in which the heterocyclyl is pyrrolyl, pyrrolidinyl imidazolyl, imidazolidinyl, piperidinyl, piperazinyl, pyridinyl, pyrimidinyl, pyrazinyl, pyridazinyl, oxazolyl, thiazolyl, morpholinyl, thiomorpholinyl, S-oxothiomorpholinyl or S,S-dioxothiomorpholinyl wherein heterocyclyl is unsubstituted or substituted by C₁-C₇-alkyl, pyrrolidinyl, piperazinyl, amino, N-mono- and/or N,N-di-C₁-C₇-alkylamino, halo, hydroxyl, C₁-C₇-alkoxy methoxy, oxo and/or halo-C₁-C₇-alkyl;
C₃-C₈-cyloalkoxy;
pyridincarbonylamino-C₁-C₇-alkoxy, C₆-C₁₄-arylaminocarbonylamino-C₂-C₇-alkoxy wherein C₆-C₁₄-aryl is unsubstituted or substituted by one to three substituents independently selected from the group consisting of C₁-C₇-alkyl, halo-C₁-C₇-alkyl, hydroxy, C₁-C₇-alkoxy, halo and cyano;
pyridinylaminocarbonylamino-C₁-C₇-alkoxy;
C₁-C₇-alkanoyloxy;
benzoyloxy- or naphthoyloxy;
amino;
mono- or di-(C₁-C₇-alkyl, C₃-C₈-cyloalkyl and/or hydroxyl-C₁-C₇-alkyl)-amino;
mono- or di-(naphthyl- or phenyl-C₁-C₇-alkyl)-amino;
C₁-C₇-alkanoylamino;
unsubstituted or amino-, N-mono- or N,N-di-(C₁-C₇-alkyl and/or phenyl- or naphthyl-C₁-C₇-alkyl)amino-substituted benzoylamino- or naphthoylamino;
C₁-C₇-alkoxycarbonylamino;
(phenyl or naphthyl)-C₁-C₇-alkoxycarbonylamino;
C₁-C₇-alkylsulfonylamino;
phenyl- or naphthylsulfonylamino wherein phenyl or naphthyl is unsubstituted or substituted by one to three C₁-C₇-alkyl moieties;
phenyl- or naphthyl-C₁-C₇-alkylsulfonylamino;
C₁-C₇-alkanoyl;
unsubstituted or substituted benzoyl wherein the substituted benzoyl has one to three substitutents independently selected from the group consisting of hydroxy, C₁-C₇-alkoxy and cyano;
C₁-C₇-alkylthio;
halo-C₁-C₇-alkylthio;
C₁-C₇-alkane-sulfonyl;
C₃-C₈-cyloalkyl-sulfonyl;
C₁-C₇-alkoxy-C₁-C₇-alkylthio;
phenyl- or naphthylthio;
phenyl- or naphthyl-C₁-C₇-alkylthio;
C₁-C₇-alkanoylthio;
benzoyl- or naphthaylthio;
C₁-C₇-alkanoyl;
C₁-C₇-alkoxy-C₁-C₇-alkanoyl;
carboxyl (—COOH);
C₁-C₇-alkoxy-carbonyl;
phenoxy- or naphthoxycarbonyl;
phenyl- or naphthyl-C₁-C₇-alkoxycarbonyl;
C₁-C₄-alkylendioxy;
carbamoyl;
N-mono- or N,N-di-[C₁-C₇-alkyl, naphthyl-C₁-C₇-alkyl, phenyl-C₁-C₇-alkyl, N′-mono- or N′,N′-di-(C₁-C₇-alkyl)amino-C₁-C₇-alkyl, pyrrolidinyl C₁-C₇-alkyl, piperidinyl C₁-C₇-alkyl, piperazinyl- or N—(C₁-C₇-alkyl)piperazinyl C₁-C₇-alkyl, mono-C₁-C₇-alkoxy-C₁-C₇-alkyl, (N′-mono- or N′,N′-di-(C₁-C₇-alkyl)-amino)-C₁-C₇-alkyl, phenyl, pyridinyl, oxazolyl or thiazolyl each of which is unsubstituted or substituted by C₁-C₇-alkoxy, halo, pyrrolidino, piperidino, piperazino, hydroxyl-C₁-C₇-alkylamino, hydroxyl-C₁-C₇-alkyl, amino or by N-mono- or N,N-di-(C₁-C₇-alkyl)amino, C₃-C₈-cyloalkyl, pyrrolidinyl, piperidinyl, morpholinyl, piperazinyl, pyrimidinyl, pyrazinyl and/or pyridazinyl]-amino-carbonyl;
pyrrolidin-1-carbonyl;
amino-N-pyrrolidin-1-carbonyl;
N-mono- or N,N-di(C₁-C₇-alkyl)amino-pyrrolidin-1-carbonyl;
piperidin-1-carbonylmorpholin-4-carbonyl;
morpholinocarbonyl, thiomorpholinocarbonyl, S-oxo- or S,S-dioxo-thiomorpholino-carbonyl, thiomorpholin-4-carbonyl;
S-oxo-thiomorpholin-4-carbonyl;
S,S-dioxothiomorpholin-4-carbonyl;
piperazin-1-carbonyl;
N—C₁-C₇-alkyl-piperazin-1-carbonyl;
N—C₁-C₇-alkoxycarbonyl-piperazin-1-carbonyl;
N-mono- or N,N-di-(C₁-C₇-alkyl)-amino-substituted or unsubstituted pyrrolidinyl-C₁-C₇-alkylcarbonyl;
cyano;
C₁-C₇-alkenylene or -alkinylene;
C₁-C₇-alkylsulfonyl;
phenyl- or naphthylsulfonyl wherein phenyl or naphthyl is unsubstituted or substituted by one to three moieties independently selected from the group consisting of hydroxy, C₁-C₇-alkoxy and cyano;
phenyl- or naphthyl-C₁-C₇-alkylsulfonyl;
sulfamoyl;
Heterocyclyl-aminosulfonyl wherein the heterocyclyl is pyrrolidinyl, piperidinyl, piperazinyl, pyridinyl, pyrimidinyl, pyrazinyl, pyridazinyl, oxazolyl or thiazolyl;
pyrazolyl;
pyrazolidinyl;
pyrrolyl;
pyridinyl that is unsubstituted or substituted by C₁-C₇-alkoxy or halo-C₁-C₇-alkyl;
pyrrolidinyl;
oxo-pyrrolidinyl;
piperidinyl;
oxo-piperidinyl;
morpholinyl;
thiomorpholinyl;
S-oxo-thiomorpholinyl;
S,S-dioxothiomorpholinyl;
piperazinyl;
N—C₁-C₇-alkyl-piperazinyl;
4-(phenyl-C₁-C₇-alkyl)-piperazinyl;
4-(naphthyl-C₁-C₇-alkyl)-piperazinyl;
4-(C₁-C₇-alkoxycarbonyl)-piperazinyl;
4-(phenyl-C₁-C₇-alkoxycarbonyl)-piperazinyl;
4-(naphthyl-C₁-C₇-alkoxycarbonyl)-piperazinyl;
oxazolyl;
thiazolyl;
triazolyl, e.g. 1,2,4-triazol-1-yl;
carbamoyl-triazolyl;
pyrazolyl;
halo-C₁-C₇-alkyl-pyrazolyl;
halophenyl-pyrazolyl;
pyrimidin-(2-, 4- or 5-)yl;
benzimidazolyl;
pyrrolo-pyrimidinyl;
C₁-C₇-alkyl-substituted pyrrolo-pyrimidinyl;
1H,4H,5H -trihydropyrazolo[2,3-c]piperidin-1-yl;
nitro;
C₃-C₈-cycloalkyl, phenyl or naphthyl each of which is unsubstituted or substituted by one or two moieties independently selected from the group consisting of halo, C₁-C₇-alkoxy, C₁-C₇-alkanesulfonyl, nitro and cyano;
tetrazolyl;
indol-(e.g.5-)yl;
indazolyl;
and pyrrolo-pyridinyl, and
when R₁ is heterocyclyl, the heterocyclyl is unsaturated, saturated or partially saturated and is a monocyclic or bicyclic or tricyclic ring, has 4 to 10 ring atoms, and one to four ring atoms are heteroatoms
selected from the group consisting of nitrogen, oxygen and sulfur;
the heterocyclyl is unsubstituted or substituted by one to three substituents independently selected from the group consisting of
C₁-C₇-alkyl that is unsubstituted or substituted by hydroxyl, C₁-C₇-alkoxy, or halo
cyano-C₁-C₇-alkyl
amino- or C₁-C₇-alkylamino-C₁-C₇-alkyl,
halo,
hydroxyl,
C₁-C₇-alkoxy,
oxo,
amino,
mono- or di-(C₁-C₇-alkyl, hydroxyl-C₁-C₇-alkyl and/or C₃-C₈-cyloalkyl)-amino,
C₁-C₇-alkanoylamino,
C₁-C₇-alkoxycarbonyl-amino,
benzoylamino,
aminobenzoylamino,
C₁-C₇-alkoxycarbonylamino,
(phenyl or naphthyl)-C₁-C₇-alkoxycarbonylamino,
carbamoyl,
N mono- or N,N-di-(C₁-C₇-alkyl, phenyl-C₁-C₇-alkyl and/or C₃-C₈-cycloalkyl)-aminocarbonyl,
heterocyclyl aminocarbonyl which is unsubstituted or substituted by one or more substituents independently selected from C₁-C₇-alkyl, halo-C₁-C₇-alkyl, halophenyl, hydroxy, C₁-C₇-alkoxy, halo, C₁-C₇-alkoxycarbonyl, carbamoyl, phenylsulfonyl wherein phenyl is unsubstituted or substituted by one to three substituents independently selected from C₁-C₇-alkyl, hydroxy, C₁-C₇-alkoxy, halo, nitro and cyano, heterocyclylcarbonyl where heterocyclyl is bound via a ring nitrogen to the carbonyl, C₁-C₇-alkanesulfonyl, sulfamoyl, N-mono- or N,N-disubstituted sulfamoyl, cyano and nitro,
phenylaminocarbonyl,
N—[N′-mono- or N′,N′-di-(C₁-C₇-alkyl)-amino-C₁-C₇-alkyl]-aminocarbonyl,
mono- or di-[C₁-C₇-alkoxy, pyrrolidino, piperidino, piperazino, thiazolyl, hydroxyl-C₁-C₇-alkylamino and/or N′-mono- or N′,N′-di-(C₁-C₇-alkyl)-amino]substituted phenyl-aminocarbonyl,
heterocyclyl that is unsubstituted or substituted by one to three substituents independently selected from C₁-C₇-alkyl, halo-C₁-C₇-alkyl, halophenyl, hydroxy, C₁-C₇-alkoxy, halo, C₁-C₇-alkoxycarbonyl, carbamoyl, phenylsulfonyl wherein phenyl is unsubstituted or substituted by one to three substituents independently selected from C₁-C₇-alkyl, hydroxy, C₁-C₇-alkoxy, halo, nitro and cyano,
heterocyclylcarbonyl where heterocyclyl is bound via a ring nitrogen to the carbonyl,
C₁-C₇-alkanoyl,
unsubstituted or substituted benzoyl which is substituted with one to three substitutents independently selected from the group consisting of hydroxy, C₁-C₇-alkoxy and cyano,
C₁-C₇-alkanesulfonyl,
unsubstituted or substituted benzenesulfonyl which is substituted with one to three substitutents independently selected from the group consisting of hydroxy, C₁-C₇-alkoxy and cyano,
sulfamoyl,
N-mono- or N,N-disubstituted sulfamoyl, and
and wherein R² is substituted phenyl or substituted naphthyl that is substituted by one to three substitutents independently selected from the group consisting of C₁-C₇-alkyl, C₂-C₇-alkenyl;
C₂-C₇-alkinyl;
C₆-C₁₈-aryl-C₁-C₇-alkyl in which aryl is unsubstituted or substituted by C₁-C₇-alkyl, pyrrolidinyl, piperazinyl, amino, N-mono- and/or N,N-di-C₁-C₇-alkylamino, halo, hydroxy, C₁-C₇-alkoxy and/or halo-C₁-C₇-alkyl;
heterocyclyl C₇-alkyl wherein the heterocyclyl is selected from pyrrolidinyl, piperidinyl, piperazinyl, pyridinyl, pyrimidinyl, pyrazinyl, pyridazinyl, oxazolyl or thiazolyl and the heterocyclyl is unsubstituted or substituted by C₁-C₇-alkyl, pyrrolidinyl, piperazinyl, amino, N-mono- and/or N,N-di-C₁-C₇-alkylamino, halo, hydroxyl, C₁-C₇-alkoxy, oxo and/or halo-C₁-C₇-alkyl;
heterocyclyl oxy-C₁-C₇-alkyl wherein the heterocycle is selected from pyrrolidinyl, piperidinyl, piperazinyl, pyridinyl, pyrimidinyl, pyrazinyl, pyridazinyl, oxazolyl and thiazolyl the heterocyclyl is unsubstituted or substituted by C₁-C₇-alkyl, pyrrolidinyl, piperazinyl, amino, N-mono- and/or N,N-di-C₁-C₇-alkylamino, halo, hydroxyl, C₁-C₇-alkoxy, oxo and/or halo-C₁-C₇-alkyl;
heterocyclyl carbonyl-C₁-C₇-alkyl wherein the heterocyle is selected from pyrrolidin, piperidin, piperazin, pyridin, pyrimidin, pyridazin, oxazol or pyridazin and the heterocyclyl is unsubstituted or substituted by C₁-C₇-alkyl, pyrrolidinyl, piperazinyl, amino, N-mono- and/or N,N-di-C₁-C₇-alkylamino, halo, hydroxyl, C₁-C₇-alkoxy, oxo and/or halo-C₁-C₇-alkyl;
halo-C₁-C₇-alkyl;
hydroxy-C₁-C₇-alkyl;
C₁-C₇-alkoxy-C₁-C₇-alkyl;
C₁-C₇-alkoxy-C₁-C₇-alkoxy-C₁-C₇-alkyl;
phenyloxy- or naphthyloxy-C₁-C₇-alkyl;
phenyl-C₁-C₇-alkoxy- or naphthyl-C₁-C₇-alkoxy-C₁-C₇-alkyl;
amino-C₁-C₇-alkyl;
N-mono- or N,N-di-(C₁-C₇-alkyl, C₁-C₇-alkoxy-C₁-C₇-alkyl and/or (mono- or di-(C₁-C₇-alkyl)-amino)-C₁-C₇-alkyl)-amino-C₁-C₇-alkyl;
C₁-C₇-alkoxy-C₁-C₇-alkylamino-C₁-C₇-alkyl;
mono- or di-[C₆-C₁₈-aryl]-C₁-C₇-alkyl in which aryl is unsubstituted or substituted by C₁-C₇-alkyl, pyrrolidinyl, piperazinyl, amino, N-mono- and/or N,N-di-C₁-C₇-alkylamino, halo, hydroxyl, C₁-C₇-alkoxy and/or halo-C₁-C₇-alkyl;
(naphthyl- or phenyl-C₁-C₇-alkyl)-amino-C₁-C₇-alkyl;
C₁-C₇-alkanoylamino-C₁-C₇-alkyl;
carboxy-C₁-C₇-alkyl;
benzoyl- or naphthoylamino-C₁-C₇-alkyl;
C₁-C₇-alkylsulfonylamino-C₁-C₇-alkyl;
phenyl- or naphthylsulfonylamino-C₁-C₇-alkyl wherein phenyl or naphthyl is unsubstituted or substituted by one to three C₁-C₇-alkyl moieties;
phenyl- or naphthyl-C₁-C₇-alkylsulfonylamino-C₁-C₇-alkyl;
cyano-C₁-C₇-alkyl;
halo;
hydroxy;
C₁-C₇-alkoxy which is unsubstituted or substituted by one or more substituents selected from pyrrolidinyl, piperazinyl, amino, N-mono- and/or N,N-di-C₁-C₇-alkylamino, halo, hydroxyl, C₁-C₇-alkoxy, halo-C₁-C₇-alkyl, and/or by a cyclic ether radical selected from oxiranyl, oxetanyl, tetrahydrofuranyl or tetrahydropyranyl,
C₆-C₁₈-aryl-C₁-C₇-alkoxy in which aryl is phenyl or naphthyl and aryl is unsubstituted or substituted by C₁-C₇-alkyl, hydroxyl, C₁-C₇-alkoxy, pyrrolidinyl, piperazinyl, amino, N-mono- and/or N,N-di-C₁-C₇-alkylamino, halo, C₁-C₇-alkoxy and/or halo-C₁-C₇-alkyl;
hydroxy-C₁-C₇-alkoxy;
C₁-C₇-alkoxy-C₁-C₇-alkoxy;
C₁-C₇-alkoxy-C₁-C₇-alkoxy-C₁-C₇-alkoxy;
halo-C₁-C₇-alkoxy;
amino-C₁-C₇-alkoxy;
N-mono- or N,N-di-(C₁-C₇-alkyl)-amino-C₁-C₇-alkoxy;
N—C₁-C₇-alkanoylamino-C₁-C₇-alkoxy;
C₁-C₇-alkoxycarbonylamino-C₁-C₇-alkoxy;
C₆-C₁₄-arylcarbonylamino-C₂-C₇-alkoxy (C₆-C₁₄-aryl-C(═O)—NH—C₂-C₇-alkoxy or C₆-C₁₄-aroyl-NH—C₂-C₇-alkoxy) wherein C₆-C₁₄-aryl is unsubstituted or substituted by one to three substituents independently selected from the group consisting of C₁-C₇-alkyl, halo-C₁-C₇-alkyl, hydroxy, C₁-C₇-alkoxy, halo and cyano;
N-mono- or N,N-di-(C₁-C₇-alkyl)carbamoyl-C₁-C₇-alkoxy;
phenyl- or naphthyloxy;
phenyl- or naphthyl-C₁-C₇-alkyloxy;
heterocyclyl C₁-C₇-alkoxy wherein heterocyclyl is pyrrolidinyl, piperidinyl, piperazinyl, pyridinyl, pyrimidinyl, pyrazinyl, pyridazinyl, oxazolyl or thiazolyl, and where the heterocyclyl is unsubstituted or substituted by C₁-C₇-alkyl, pyrrolidinyl, piperazinyl, amino, N-mono- and/or N,N-di-C₁-C₇-alkylamino, halo, hydroxyl, C₁-C₇-alkoxy, oxo and/or halo-C₁-C₇-alkyl;
heterocyclyl oxy-C₁-C₇-alkoxy wherein heterocyclyl is pyrrolidinyl, piperidinyl, piperazinyl, pyridinyl, pyrimidinyl, pyrazinyl, pyridazinyl, oxazolyl or thiazolyl and wherein the heterocyclyl is unsubstituted or substituted by C₁-C₇-alkyl, pyrrolidinyl, piperazinyl, amino, N-mono- and/or N,N-di-C₁-C₇-alkylamino, halo, hydroxyl, C₁-C₇-alkoxy, oxo and/or halo-C₁-C₇-alkyl;
C₃-C₈-cyloalkoxy;
pyridincarbonylamino-C₁-C₇-alkoxy;
C₆-C₁₄-arylaminocarbonylamino-C₂-C₇-alkoxy wherein C₆-C₁₄-aryl is unsubstituted or substituted by one to three substituents independently selected from the group consisting of C₁-C₇-alkyl, halo-C₁-C₇-alkyl, hydroxy, C₁-C₇-alkoxy, halo and cyano;
pyridinylaminocarbonylamino-C₁-C₇-alkoxy;
C₁-C₇-alkanoyloxy;
benzoyl- or naphthoyloxy;
amino;
mono- or di-(C₁-C₇-alkyl, C₃-C₈-cyloalkyl and/or hydroxyl-C₁-C₇-alkyl)-amino;
mono- or di-(naphthyl- or phenyl-C₁-C₇-alkyl)-amino;
C₁-C₇-alkanoylamino;
benzoyl- or naphthoylamino, each of which is unsubstituted or substituted with amino-, N-mono- or N,N-di-(C₁-C₇-alkyl and/or phenyl- or naphthyl-C₁-C₇-alkyl)amino;
C₁-C₇-alkoxycarbonylamino;
(phenyl or naphthyl)-C₁-C₇-alkoxycarbonylamino;
C₁-C₇-alkylsulfonylamino;
phenyl- or naphthylsulfonylamino wherein phenyl or naphthyl is unsubstituted or substituted by one to three C₁-C₇-alkyl moieties;
phenyl- or naphthyl-C₁-C₇-alkylsulfonylamino;
C₁-C₇-alkanoyl;
unsubstituted or substituted benzoyl wherein the substitutents are one to three substitutents independently selected from the group consisting of hydroxy, C₁-C₇-alkoxy and cyano;
C₁-C₇-alkylthio;
halo-C₁-C₇-alkylthio;
C₁-C₇-alkane-sulfonyl;
C₃-C₈-cyloalkyl-sulfonyl;
C₁-C₇-alkoxy-C₁-C₇-alkylthio;
phenyl- or naphthylthio;
phenyl- or naphthyl-C₁-C₇-alkylthio;
C₁-C₇-alkanoylthio;
benzoyl- or naphthaylthio;
C₁-C₇-alkanoyl;
C₁-C₇-alkoxy-C₁-C₇-alkanoyl;
carboxyl;
C₁-C₇-alkoxy-carbonyl;
phenoxy- or naphthoxycarbonyl;
phenyl- or naphthyl-C₁-C₇-alkoxycarbonyl;
C₁-C₄-alkylendioxy;
carbamoyl;
N-mono- or N,N-di-[C₁-C₇-alkyl, naphthyl-C₁-C₇-alkyl, phenyl-C₁-C₇-alkyl, N′-mono- or N′,N′-di-(C₁-C₇-alkyl)amino-C₁-C₇-alkyl, pyrrolidinyl-C₁-C₇-alkyl, piperidinyl-C₁-C₇-alkyl, piperazinyl- or N—(C₁-C₇-alkyl)piperazinyl-C₁-C₇-alkyl, mono-C₁-C₇-alkoxy-C₁-C₇-alkyl, (N′-mono- or N′,N′-di-(C₁-C₇-alkyl)-amino)-C₁-C₇-alkyl, phenyl, pyridinyl, oxazolyl or thiazolyl each of which is unsubstituted or substituted by C₁-C₇-alkoxy, halo, pyrrolidino, piperidino, piperazino, hydroxyl-C₁-C₇-alkylamino, hydroxyl-C₁-C₇-alkyl, amino or N-mono- or N,N-di-(C₁-C₇-alkyl)-amino, C₃-C₈-cyloalkyl, pyrrolidinyl, piperidinyl, morpholinyl, piperazinyl, pyrimidinyl, pyrazinyl and/or pyridazinyl-amino-carbonyl;
N—C₁-C₇-alkoxy-C₁-C₇-alkylcarbamoyl;
pyrrolidin-1-carbonyl;
amino-N-pyrrolidin-1-carbonyl;
N-mono- or N,N-di(C₁-C₇-alkyl)amino-pyrrolidin-1-carbonyl;
piperidin-1-carbonylmorpholin-4-carbonyl;
morpholinocarbonyl, thiomorpholinocarbonyl, S-oxo- or S,S-dioxo-thiomorpholino-carbonyl, thiomorpholin-4-carbonyl;
S-oxo-thiomorpholin-4-carbonyl;
S,S-dioxothiomorpholin-4-carbonyl;
piperazin-1-carbonyl;
N—C₁-C₇-alkyl-piperazin-1-carbonyl;
N—C₁-C₇-alkoxycarbonyl-piperazin-1-carbonyl;
N-mono- or N,N-di-(C₁-C₇-alkyl)-amino-substituted or unsubstituted pyrrolidinyl-C₁-C₇-alkylcarbonyl;
cyano;
C₁-C₇-alkenylene or -alkinylene;
C₁-C₇-alkylsulfonyl;
phenyl- or naphthylsulfonyl wherein phenyl or naphthyl is unsubstituted or substituted by one to three moieties independently selected from the group consisting of C₁-C₇-alkyl, hydroxy, C₁-C₇-alkoxy and cyano;
phenyl- or naphthyl-C₁-C₇-alkylsulfonyl;
sulfamoyl;
N-mono or N,N-di-[C₁-C₇-alkyl, phenyl-, naphthyl-, phenyl-C₁-C₇-alkyl-, pyrrolidinyl-C₁-C₇-alkyl, piperidinyl-C₁-C₇-alkyl, piperazinyl-C₁-C₇-alkyl, N—C₁-C₇-alkylpiperazinyl-C₁-C₇-alkyl, naphthyl-C₁-C₇-alkyl, phenyl which is unsubstituted or substituted by C₁-C₇-alkoxy, halo, pyrrolidino, piperidino, piperazino, hydroxyl-C₁-C₇-alkyl or N-mono- or N,N-di-(C₁-C₇-alkyl)-C₁-C₇-alkyl, pyrrolidinyl, piperidinyl piperazinyl, pyridinyl, pyrimidinyl, pyrazinyl, pyridazinyl, oxazolyl and/or thiazolyl]-aminosulfonyl;
pyrazolyl;
pyrazolidinyl;
pyrrolyl;
pyridinyl that is unsubstituted or substituted by C₁-C₇-alkoxy and/or by halo-C₁-C₇-alkyl;
pyrrolidinyl;
oxo-pyrrolidinyl;
piperidinyl;
oxo-piperidinyl;
morpholinyl;
thiomorpholinyl;
S-oxo-thiomorpholinyl;
S,S-dioxothiomorpholinyl;
piperazinyl;
N—C₁-C₇-alkyl-piperazinyl;
4-(phenyl-C₁-C₇-alkyl)-piperazinyl;
4-(naphthyl-C₁-C₇-alkyl)-piperazinyl;
4-(C₁-C₇-alkoxycarbonyl)-piperazinyl;
4-(phenyl-C₁-C₇-alkoxycarbonyl)-piperazinyl;
4-(naphthyl-C₁-C₇-alkoxycarbonyl)-piperazinyl;
oxazolyl;
thiazolyl;
triazolyl;
carbamoyl-triazolyl;
pyrazolyl;
halo-C₁-C₇-alkyl-pyrazolyl;
halophenyl-pyrazolyl;
pyrimidin-yl;
benzimidazolyl;
C₁-C₇-alkoxy-substituted benzimidazolyl;
pyrrolo-pyrimidinyl;
C₁-C₇-alkyl-substituted pyrrolo-pyrimidinyl;
1H,4H,5H-trihydropyrazolo[2,3-c]piperidin-1-yl which is unsubstituted or substituted by 1 or 2 substituents independently selected from C₁-C₇-alkyl, halo-C₁-C₇-alkyl, nitro C₃-C₈-cycloalkyl, phenyl or naphthyl each of which is unsubstituted or substituted by one to 2 moieties independently selected from the group consisting of halo, C₁-C₇-alkoxy, C₁-C₇-alkanesulfonyl, nitro and cyano;
tetrazolyl;
indolyl;
indazolyl;
C₁-C₇-alkyl-indazolyl;
and pyrrolo-pyridinyl;
or an N-oxide thereof, a solvate and/or a salt thereof.

3. A compound of the formula I according to claim 2, wherein
R² is substituted phenyl with two substituents of which one substituent is a meta C₁-C₇-alkoxy and the other substituent is selected from the list of substituents for R² defined in claim 2;
or an N-oxide thereof, a solvate and/or a salt thereof.

4. (canceled)

5. A compound of the formula I according to claim 1, wherein
R¹ is phenyl, pyridinyl, pyrimidinyl, pyrazinyl, pyrazolyl or imidazolyl, each of which is unsubstituted or substituted by one or two substituents independently selected from the group consisting of C₁-C₇-alkyl, halo-C₁-C₇-alkyl, hydroxy, C₁-C₇-alkoxy, halo, amino, C₁-C₇-alkoxycarbonylamino, pyridinyl that is unsubstituted or substituted by one or two moieties independently selected from the group consisting of C₁-C₇-alkyl, hydroxy, C₁-C₇-alkoxy, halo, amino, C₁-C₇-alkoxycarbonylamino and cyano, piperidinyl, 1-(C₁-C₇-alkoxy)-piperidin-4-yl, piperazino, 4-(C₁-C₇-alkoxycarbonyl)-piperazino, morpholino, thiomorpholino, S-oxo or S,S-dioxothiomorpholino, (unsubstituted or cyano- and/or hydroxy-substituted-phenyl)-sulfonyl, cyano C₁-C₇-alkane-sulfonyl and C₃-C₈-cyloalkyl-sulfonyl; and
R² is phenyl or napthyl, each of which is unsubstituted or substituted by one or two substitutents selected from the group consisting of C₁-C₇-alkyl, phenyl that is unsubstituted or substituted by one to three moieties independently selected from hydroxy and C₁-C₇-alkoxy, C₁-C₇-alkoxy, hydroxy-C₂-C₇-alkoxy, C₁-C₇-alkoxy-C₂-C₇-alkoxy, (C₁-C₇-alkoxy-C₂-C₇-alkoxy)-C₂-C₇-alkoxy, amino-C₁-C₇-alkoxy, N-mono- or N,N-di-(C₁-C₇-alkyl)amino-C₁-C₇-alkoxy, C₁-C₇-alkoxycarbonylamino-C₂-C₇-alkoxy, C₁-C₇-alkanoylamino-C₁-C₇-alkoxy, C₆-C₁₄-arylcarbonylamino-C₁-C₇-alkoxy wherein C₆-C₁₄-aryl is unsubstituted or substituted by one to three substituents independently selected from the group consisting of C₁-C₇-alkyl, halo-C₁-C₇-alkyl, hydroxy, C₁-C₇-alkoxy, halo, especially fluoro, and cyano, pyridincarbonylamino-C₂-C₇-alkoxy, C₁-C₇-alkylaminocarbonylamino-C₁-C₇-alkoxy, C₆-C₁₄-arylaminocarbonylamino-C₂-C₇-alkoxy (C₆-C₁₄-aryl-NH—C(═O)—NH—C₂-C₇-alkoxy) wherein C₆-C₁₄-aryl is unsubstituted or substituted by one or more, especially up to three, substituents independently selected from the group consisting of C₁-C₇-alkyl, halo-C₁-C₇-alkyl, hydroxy, C₁-C₇-alkoxy, halo, especially fluoro, and cyano, pyridinylaminocarbonylamino-C₂-C₇-alkoxy, pyrrolyl-C₁-C₇-alkoxy, pyrrolidinyl-C₁-C₇-alkoxy wherein pyrrolidinyl is unsubstituted or substituted by oxo, imidazolyl-C₁-C₇-alkoxy, imidazolidinyl-C₁-C₇-alkoxy wherein imidazolidinyl is unsubstituted or substituted by oxo, morpholinyl-C₁-C₇-alkoxy, thiomorpholinyl-C₁-C₇-alkoxy, S-oxothiomorpholinyl, S,S-dioxothiomorpholinyl, piperidinyl-C₁-C₇-alkoxy wherein piperidinyl is unsubstituted or substituted with C₁-C₇-alkyl, piperazinyl-C₁-C₇-alkoxy wherein piperazinyl is unsubstituted or substituted with C₁-C₇-alkyl, halo, C₁-C₇-alkylsulfonyl, nitro, cyano and from C₁-C₇-alkoxy which is unsubstituted or substituted by one pyrrolidino, piperazino, amino, N-mono- and/or N,N-di-C₁-C₇-alkylamino, halo, hydroxyl, C₁-C₇-alkoxy, halo-C₁-C₇-alkyl, oxiranyl, oxetanyl, tetrahydrofuranyl or tetrahydropyranyl,
or an N-oxide thereof, a solvate and/or a salt thereof.

6. A compound of the formula I according to claim 1 selected from the group of compounds with the following names:
6-(3,4-dimethoxy-phenyl)-3-(6-fluoro-pyridin-3-yl)-2-methyl-imidazo[1,2-b]pyridazine;
6-(3,4-dimethoxy-phenyl)-2-methyl-3-pyridin-3-yl-imidazo[1,2-b]pyridazine;
(3,4-dimethoxy-phenyl)-3-(6-methoxy-pyridin-3-yl)-2-methyl-imidazo[1,2-b]pyridazine;
5-[6-(3,4-dimethoxy-phenyl)-2-methyl-imidazo[1,2-b]pyridazin-3-yl]-pyridine-2-carbonitrile;
6-(3,4-dimethoxy-phenyl)-2-methyl-3-(6-morpholin-4-yl-pyridin-3-yl)-imidazo[1,2-b]pyridazine;
5-[6-(3,4-dimethoxy-phenyl)-2-methyl-imidazo[1,2-b]pyridazin-3-yl]-nicotinonitrile;
5-[6-(3,4-dimethoxy-phenyl)-2-methyl-imidazo[1,2-b]pyridazin-3-yl]-pyridin-2-ylamine;
5-[6-(3,4-dimethoxy-phenyl)-2-methyl-imidazo[1,2-b]pyridazin-3-yl)-pyridin-2-ol;
4-{5-[6-(3,4-dimethoxy-phenyl)-2-methyl-imidazo[1,2-b]pyridazin-3-yl]-pyridin-2-yl}-piperazine-1-carboxylic acid tert-butyl ester;
3-(6-chloro-pyridin-3-yl)-6-(3,4-dimethoxy-phenyl)-2-methyl-imidazo[1,2-b]pyridazine;
6-(3,4-dimethoxy-phenyl)-2-methyl-3-(6-piperazin-1-yl-pyridin-3-yl)-imidazo[1,2-b]pyridazine;
5-[6-(3,4-dimethoxy-phenyl)-2-methyl-imidazo[1,2-b]pyridazin-3-yl]-[2,3]bipyridinyl-6′-carbonitrile;
5-[6-(4-ethoxy-3-methoxy-phenyl)-2-methyl-imidazo[1,2-b]pyridazin-3-yl]-[2,3]bipyridinyl-6′-carbonitrile;
3-(6-chloro-pyridin-3-yl)-6-(4-ethoxy-3-methoxy-phenyl)-2-methyl-imidazo[1,2-b]pyridazine;
5-[6-(4-ethoxy-3-methoxy-phenyl)-2-methyl-imidazo[1,2-b]pyridazin-3-yl]-pyridin-2-ol;
6-(4-ethoxy-3-methoxy-phenyl)-2-methyl-3-(6-morpholin-4-yl-pyridin-3-yl)-imidazo[1,2-b]pyridazine;
5-[6-(3,4-dimethoxy-phenyl)-2-methyl-imidazo[1,2-b]pyridazin-3-yl]-3-trifluoromethyl-pyridin-2-ylamine;
6-(3,4-dimethoxy-phenyl)-2-methyl-3-(3-methyl-pyridin-2-yl)-imidazo[1,2-b]pyridazine;
6-(3,4-dimethoxy-phenyl)-2-methyl-3-pyrazin-2-yl-imidazo[1,2-b]pyridazine;
6-(4′-methoxy-biphenyl-4-yl)-2-methyl-3-(6-morpholin-4-yl-pyridin-3-yl)-imidazo[1,2-b]pyridazine;
5-[6-(4-methanesulfonyl-phenyl)-2-methyl-imidazo[1,2-b]pyridazin-3-yl]-nicotinonitrile;
6-(3,4-dimethoxy-phenyl)-2-methyl-3-(1H-pyrazol-3-yl)-imidazo[1,2-b]pyridazine;
4-{3-[6-(3,4-dimethoxy-phenyl)-2-methyl-imidazo[1,2-b]pyridazin-3-yl]-pyrazole-1-sulfonyl}-benzonitrile;
6-(3,4-dimethoxy-phenyl)-3-[1-(4-methoxy-benzenesulfonyl)-1H-pyrazol-3-yl]-2-methyl-imidazo[1,2-b]pyridazine;
3-{3-[6-(3,4-dimethoxy-phenyl)-2-methyl-imidazo[1,2-b]pyridazin-3-yl]-pyrazole-1-sulfonyl}-benzonitrile;
5-{6-[4-(2-amino-ethoxy)-3-methoxy-phenyl]-2-methyl-imidazo[1,2-b]pyridazin-3-yl}-3-trifluoromethyl-pyridin-2-ylamine, and
(2-{4-[3-(6-amino-5-trifluoromethyl-pyridin-3-yl)-2-methyl-imidazo[1,2-b]pyridazin-6-yl]-2-methoxy-phenoxy}-ethyl)-carbamic acid tert-butyl ester
or an N-oxide thereof, a solvate and/or a pharmaceutically acceptable salt thereof.

7. A compound of the formula I according to claim 1, selected from the group of compounds numbered 26 to 116 with the formulae and the substituents given in the following tables:

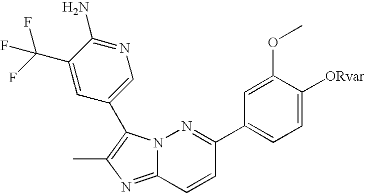

| Example | Rvar |
|---|---|
| 26 | 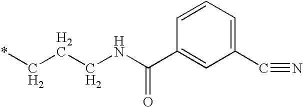 |
| 27 | 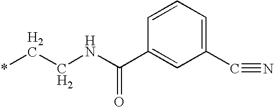 |
| 28 | 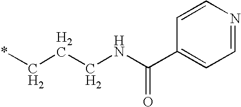 |
| 29 | 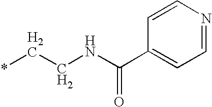 |
| 30 | 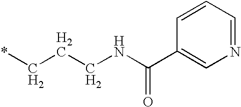 |
| 31 | 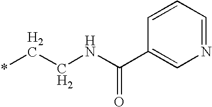 |
| 32 | 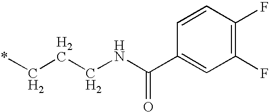 |
| 33 | 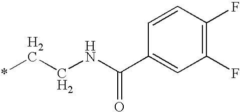 |
| 34 | 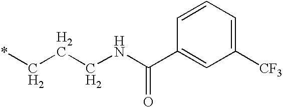 |
| 35 | 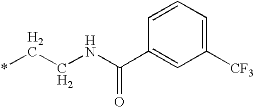 |
| 36 | 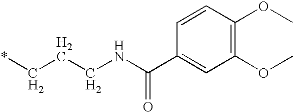 |
| 37 | 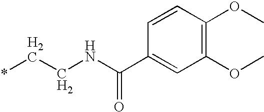 |
| 38 | 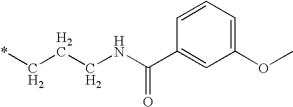 |
| 39 | 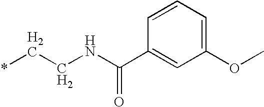 |
| 40 | 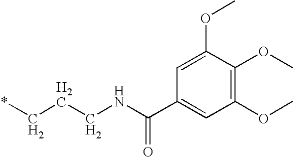 |
| 41 | 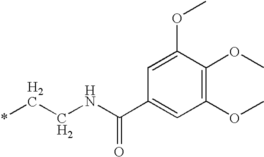 |
| 42 | 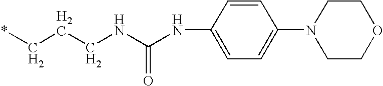 |
| 43 | 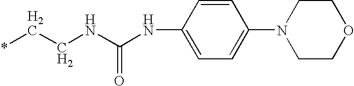 |
| 44 | 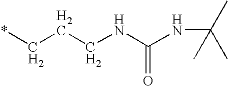 |
| 45 | 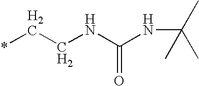 |
| 46 | 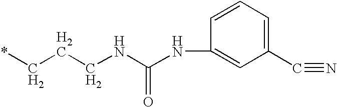 |
| 47 | 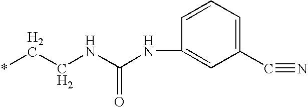 |
| 48 | 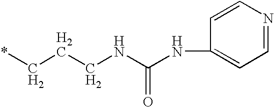 |
| 49 | 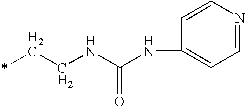 |
| 50 | 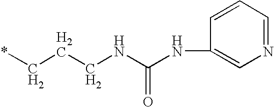 |
| 51 | 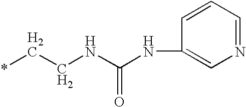 |
| 52 | 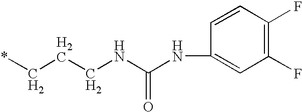 |
| 53 | 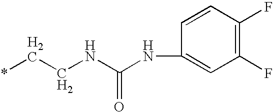 |
| 54 | 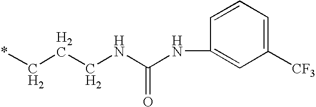 |
| 55 | 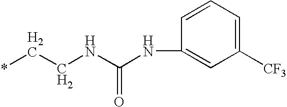 |
| 56 | 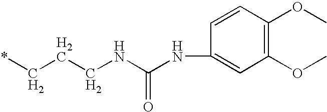 |
| 57 | 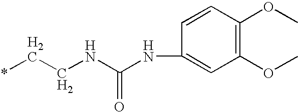 |
| 58 | 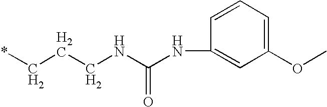 |
| 59 | 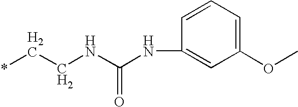 |
| 60 | 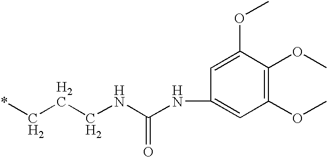 |
| 61 | 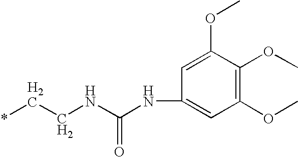 |
| 62 | 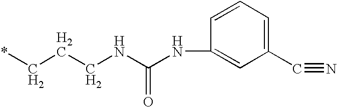 |
| 63 | 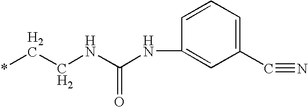 |
| 64 | 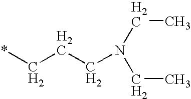 |
| 65 | 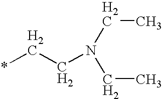 |
| 66 | 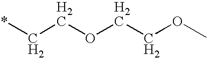 |
| 67 | 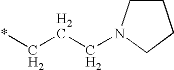 |
| 68 | 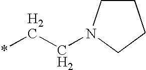 |
| 69 | 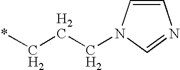 |
| 70 | 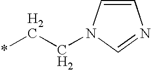 |
| 71 | 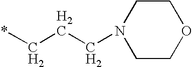 |
| 72 | 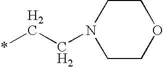 |
| 73 | 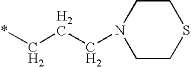 |
| 74 | 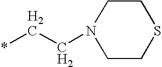 |
| 75 | 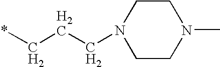 |
| 76 | 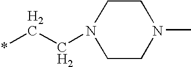 |
| 77 | 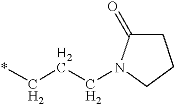 |
| 78 | 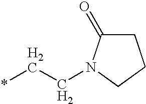 |
| 79 | 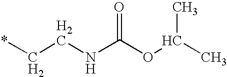 |
| 80 | 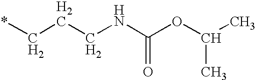 |
| 81 | 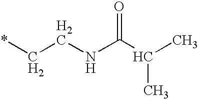 |
| 82 | 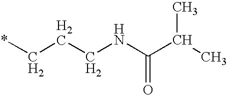 |
| EXAMPLE | structure |
| 83 | 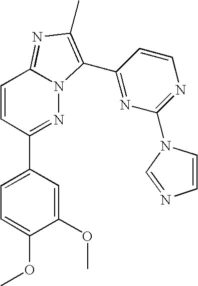 |
| 84 | 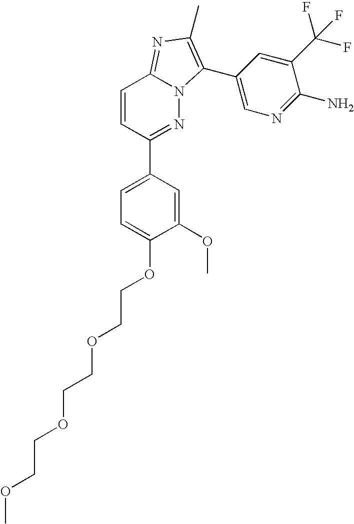 |
| 85 | 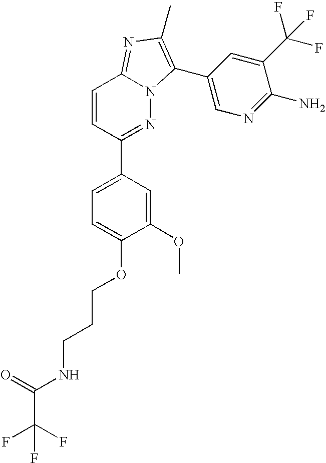 |
| 86 | 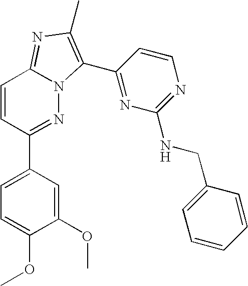 |
| 87 | 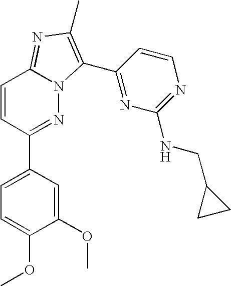 |
| 88 | 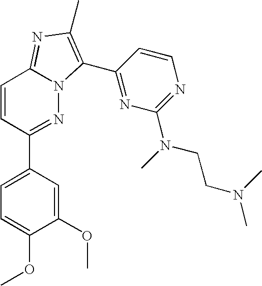 |
| 89 | 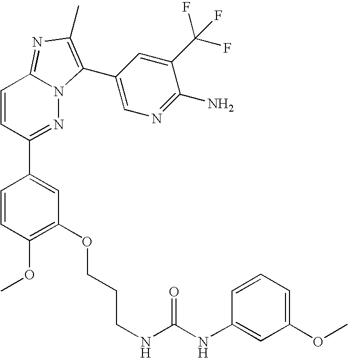 |
| 90 | 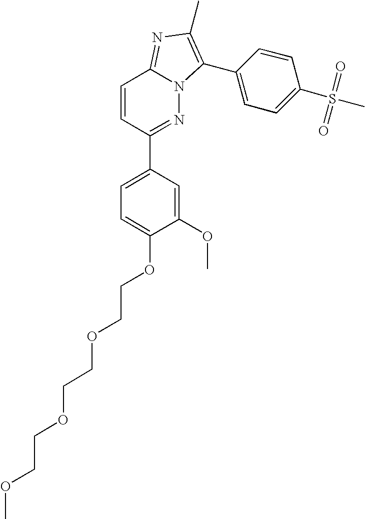 |
| 91 | 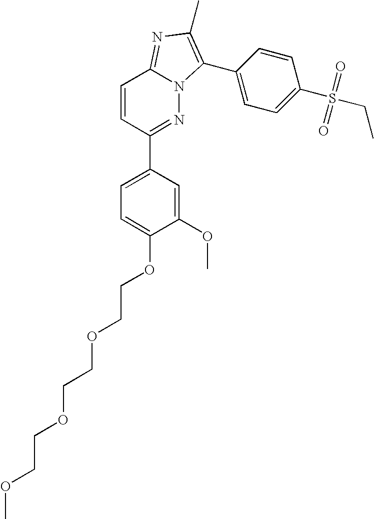 |
| 92 | 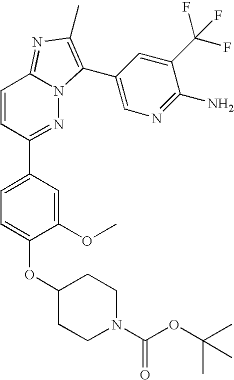 |
| 93 | 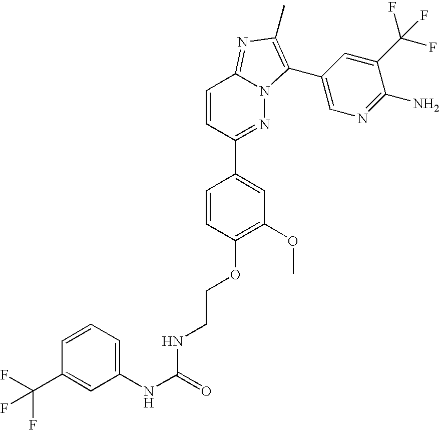 |
| 94 | 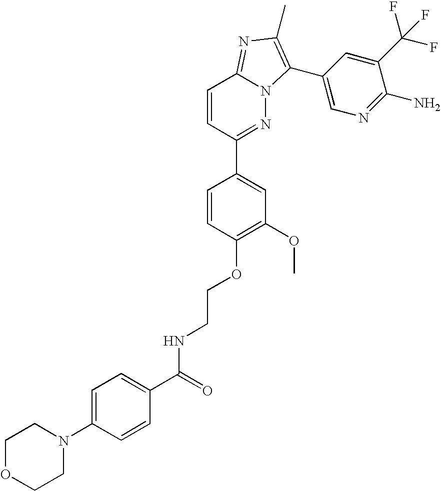 |
| 95 | 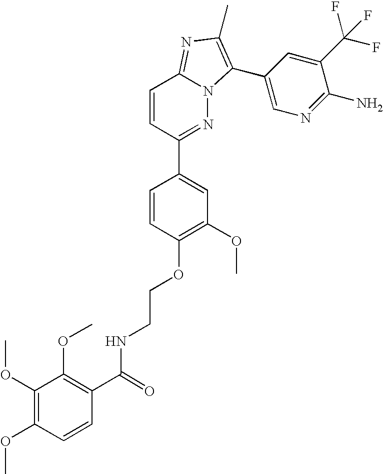 |
| 96 | 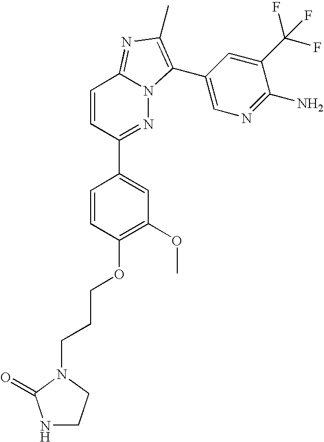 |
| 97 | 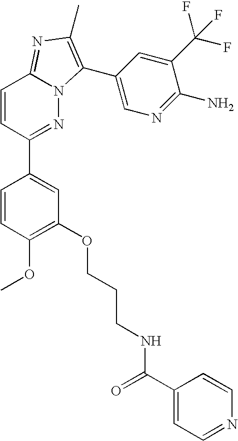 |
| 98 | 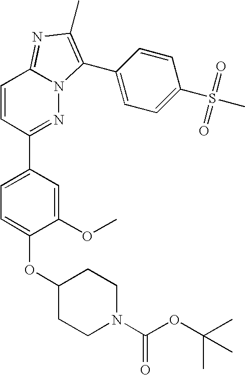 |
| 99 | 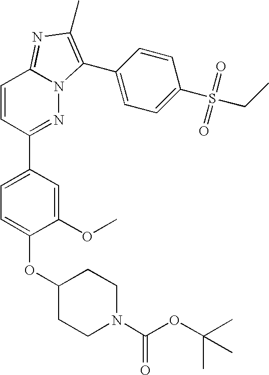 |
| 100 | 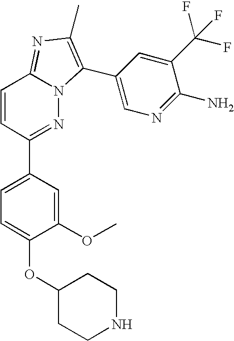 |
| 101 | 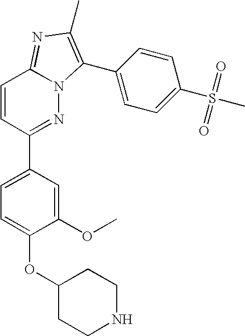 |
| 102 | 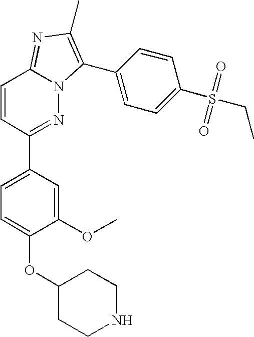 |
| 103 | 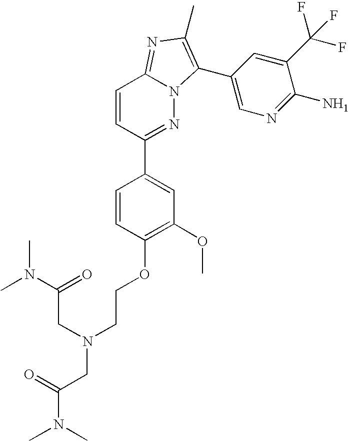 |
| 104 | 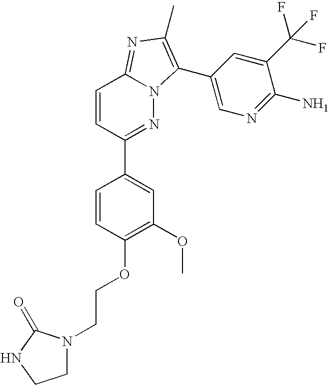 |
| 105 | 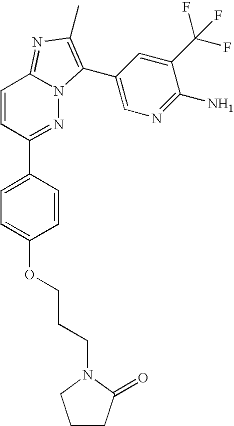 |
| 106 | 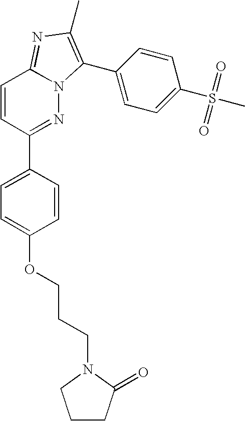 |
| 107 | 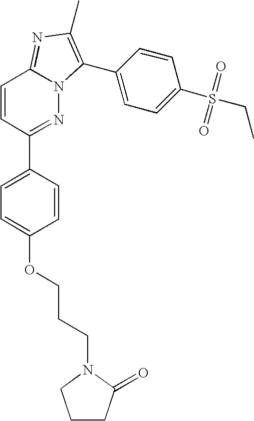 |
| 108 | 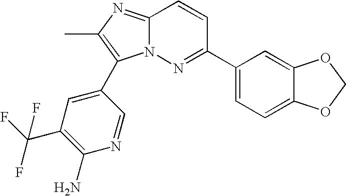 |
| 109 | 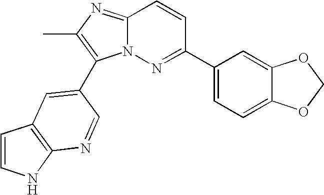 |
| 110 | 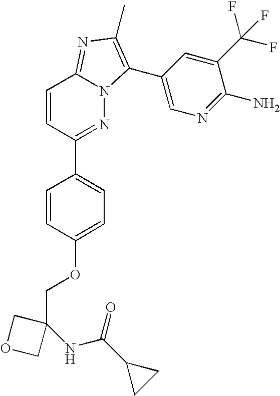 |
| 111 | 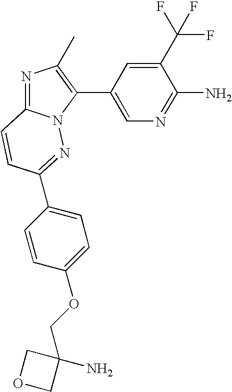 |
| 112 | 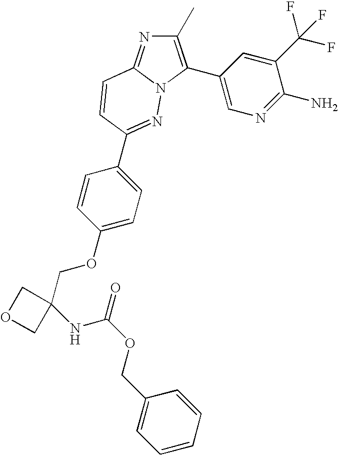 |
| 113 | 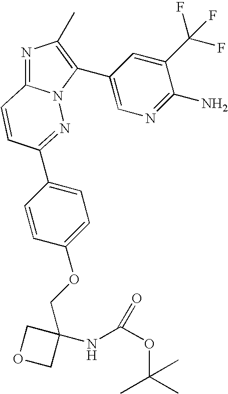 |
| 114 | 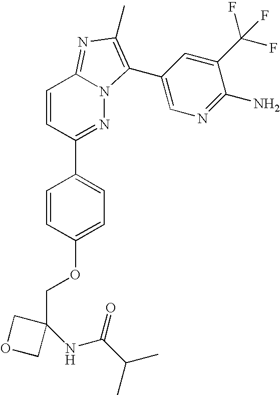 |
| 115 | 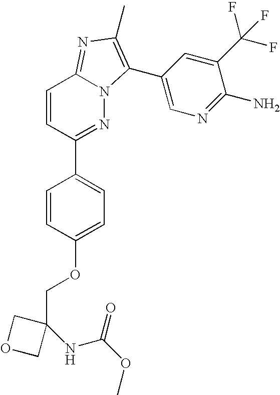 |
| 116 | 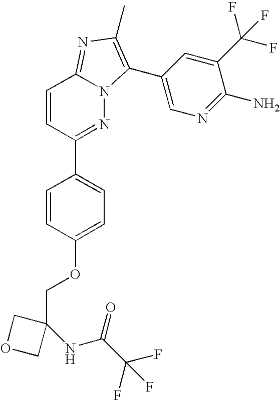 |

or an N-oxide thereof, a solvate and/or a pharmaceutically acceptable salt thereof.

8. (canceled)

9. A method of treating a disease in a warm-blooded animal wherein the disease is selected from the group consisting of proliferative; inflammatory diseases, allergic diseases, obstructive airways diseases, and disorders commonly occurring in connection with transplantation, the method comprising a stop of administering a compound of claim 1, or salt thereof, to the animal.

10. A pharmaceutical preparation, comprising a compound of the formula I, an N-oxide thereof, a tautomer thereof and/or a pharmaceutically acceptable salt thereof, according to claim 1 and at least one pharmaceutically acceptable carrier.

11. A method or process for the manufacture of a pharmaceutical preparation, comprising mixing a compound of the formula I, an N-oxide thereof, a tautomer thereof and/or a pharmaceutically acceptable salt thereof, according to claim 1 with at least one pharmaceutically acceptable carrier material.

12. A process for the manufacture of a compound according to claim 1,
said process comprising
a) reacting a compound of the formula II, 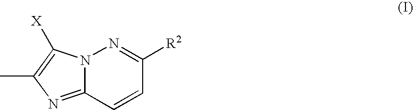
wherein R² is as defined for a compound of the formula I and X is halo, preferably chloro, bromo or iodo, or is trifluoromethansulfonyloxy, under cross-coupling conditions with a boronic acid or boronic acid ester or an organotin compound of the formula III,
R¹-D (III)
wherein R¹ is as defined for a compound of the formula I and is bound via a carbon atom to D and D is —B(OH₂) in free form or in esterified form, e.g. as dialkoxy ester or as a group of the formula A, 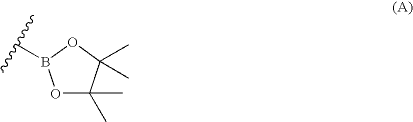
or is —Sn(alk)₃ wherein alk is alkyl, preferably C₁-C₇-alkyl, more preferably methyl,
or
b) reacting a boronic acid or boronic acid ester or organotin compound of the formula IV, 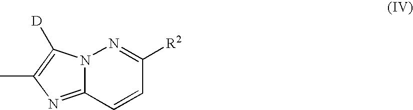
wherein R² is as defined for a compound of the formula I and D is —B(OH₂) in free form or in esterified form, e.g. as a group of the formula A shown under a), or is —Sn(alk)₃ wherein alk is alkyl, preferably C₁-C₇-alkyl, more preferably methyl, under cross-coupling conditions with a compound of the formula V,
R¹—X (V)
wherein R¹ is as defined for a compound of the formula I and X is halogen, especially chloro, bromo or iodo, or trifluoromethansulfonyloxy,
or
c) reacting a compound of the formula VI, 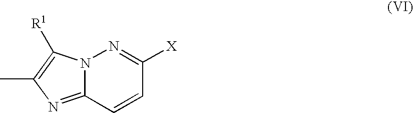
wherein R¹ is as defined for a compound of the formula I and X is halo, especially chloro, bromo or iodo, or is trifluoromethansulfonyloxy, under cross-coupling conditions with a boronic acid or boronic acid ester or organotin compound of the formula VII,
R²-D (VII)
wherein R² is as defined for a compound of the formula I and D is —B(OH₂) in free form or in esterified form, e.g. as a group of the formula A shown under a), or is —Sn(alk)₃ wherein alk is alkyl, preferably C₁-C₇-alkyl, more preferably methyl,
or
d) reacting a pyridazine compound of the formula VIII, 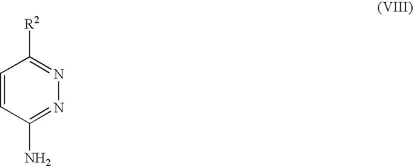
wherein R² is as defined for a compound of the formula I, with a haloketone of the formula IX, 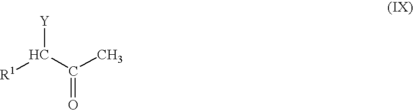
wherein R¹ is as defined for a compound of the formula I and Y is halo, especially chloro or bromo,
or
e) for the manufacture of a compound of the formula I wherein R¹ is pyrazol-3-yl, reacting a compound of the formula X, 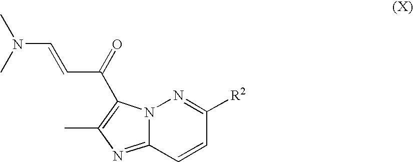
wherein R² is as defined for a compound of the formula I, with hydrazine or a hydrate and/or salt thereof,
and, if desired, a compound of the formula I obtainable according to any one of the reactions a) to e) given above is converted into a different compound of the formula I, an obtainable salt of a compound of the formula I is converted into a different salt thereof, an obtainable free compound of the formula I is converted, into a salt thereof, and/or an obtainable isomer of a compound of the formula I is separated from one or more different obtainable isomers of the formula I.
